SEQUENCE LISTING (64 sequences)

SEQ ID NO: 1 - protein, 117 aa, synthetic construct
QIQLVQSGPELKKPGETVKISCKASGYTFTNYGMNWVKQAPGKGLKWIGWINTYTGDPTYADDFKGRFAFSLETSASNGYLQIDNLKKEDTATYFCARRDIFAMDYWGQGTSVTVSS

SEQ ID NO: 2 - protein, 107 aa, synthetic construct
DIQMTQTTSSLSASLGDRVTITCRASQDIMKYLHWYQQKPDGTIKLLIYYTSRLHSGVPSRFSGSGSGTDYSLTISNLEQEDIATYFCQQDVKYPWTFGGGTKLEIK

SEQ ID NO: 3 - protein, 5 aa, synthetic construct
NYGMN

SEQ ID NO: 4 - protein, 17 aa, synthetic construct
WINTYTGDPTYADDFKG

SEQ ID NO: 5 - protein, 8 aa, synthetic construct
RDIFAMDY

SEQ ID NO: 6 - protein, 11 aa, synthetic construct
RASQDIMKYLH

SEQ ID NO: 7 - protein, 7 aa, synthetic construct
YTSRLHS

SEQ ID NO: 8 - protein, 9 aa, synthetic construct
QQDVKYPWT

SEQ ID NO: 9 - protein, 120 aa, synthetic construct
QVQLQQPGTELVKPGASVKLSCKASGYTFTNYWMHWVKQRPGQGLEWIGNINPFNGATNYNDNFKTKATLTVDKSSSTAYMHLSSLTSEDSAVYYCARYDYSKYYAMDYWGQGTSVTVSS

SEQ ID NO: 10 - protein, 108 aa, synthetic construct
ENVLTQSPAIMAASPGDKVTMTCSASSSINSAHFHWYQQKPGTSPKLWIYRTSNLASGVPARFSGRGSGTSYSLTISSMEAADAATYYCQQWSDYPWTFGGGTKLEIR

SEQ ID NO: 11 - protein, 5 aa, synthetic construct
NYWMH

SEQ ID NO: 12 - protein, 17 aa, synthetic construct
NINPFNGATNYNDNFKT

SEQ ID NO: 13 - protein, 11 aa, synthetic construct
YDYSKYYAMDY

SEQ ID NO: 14 - protein, 12 aa, synthetic construct
SASSSINSAHFH

SEQ ID NO: 15 - protein, 7 aa, synthetic construct
RTSNLAS

SEQ ID NO: 16 - protein, 9 aa, synthetic construct
QQWSDYPWT

SEQ ID NO: 17 - protein, 388 aa, Homo sapiens
MKWMVVVLVCLQLLEAAVVKVPLKKFKSIRETMKEKGLLGEFLRTHKYDPAWKYRFGDLSVTYEPMAYMDAAYFGEISIGTPPQNFLVLFDTGSSNLWVPSVYCQSQACTSHSRFNPSESSTYSTNGQTFSLQYGSGSLTGFFGYDTLTVQSIQVPNQEFGLSENEPGTNFVYAQFDGIMGLAYPALSVDEATTAMQGMVQEGALTSPVFSVYLSNQQGSSGGAVVFGGVDSSLYTGQIYWAPVTQELYWQIGIEEFLIGGQASGWCSEGCQAIVDTGTSLLTVPQQYMSALLQATGAQEDEYGQFLVNCNSIQNLPSLTFIINGVEFPLPPSSYILSNNGYCTVGVEPTYLSSQNGQPLWILGDVFLRSYYSVYDLGNNRVGFATAA

SEQ ID NO: 18 - protein, 23 aa, synthetic construct
DIQMTQTTSSLSASLGDRVTITC

SEQ ID NO: 19 - protein, 15 aa, synthetic construct
WYQQKPDGTIKLLIY

SEQ ID NO: 20 - protein, 33 aa, synthetic construct
SGVPSRFSGSGSGTDYSLTISNLEQEDIATYFC

SEQ ID NO: 21 - protein, 10 aa, synthetic construct
FGGGTKLEIK

SEQ ID NO: 22 - protein, 30 aa, synthetic construct
QIQLVQSGPELKKPGETVKISCKASGYTFT

SEQ ID NO: 23 - protein, 14 aa, synthetic construct
WVKQAPGKGLKWIG

SEQ ID NO: 24 - protein, 32 aa, synthetic construct
RFAFSLETSASNGYLQIDNLKKEDTATYFCAR

SEQ ID NO: 25 - protein, 11 aa, synthetic construct
WGQGTSVTVSS

SEQ ID NO: 26 - protein, 23 aa, synthetic construct
ENVLTQSPAIMAASPGDKVTMTC

SEQ ID NO: 27 - protein, 15 aa, synthetic construct
WYQQKPGTSPKLWIY

SEQ ID NO: 28 - protein, 32 aa, synthetic construct
GVPARFSGRGSGTSYSLTISSMEAADAATYYC

SEQ ID NO: 29 - protein, 10 aa, synthetic construct
FGGGTKLEIR

SEQ ID NO: 30 - protein, 30 aa, synthetic construct
QVQLQQPGTELVKPGASVKLSCKASGYTFT

SEQ ID NO: 31 - protein, 14 aa, synthetic construct
WVKQRPGQGLEWIG

SEQ ID NO: 32 - protein, 32 aa, synthetic construct
KATLTVDKSSSTAYMHLSSLTSEDSAVYYCAR

SEQ ID NO: 33 - protein, 11 aa, synthetic construct
WGQGTSVTVSS

SEQ ID NO: 34 - protein, 121 aa, synthetic construct
QIQLVQSGPELKKPGETVKISCKASGYTFTNYGMNWVKQAPGKGLKWMGLINTYTRGPTFADDFKGRFAFSVETSANTAYLQINNLKTEDTATYFCARRGEYYGSSYVDYWGQGTTLTVSS

SEQ ID NO: 35 - protein, 107 aa, synthetic construct
DIQMTQITSSLSASLGDRVTISCRASQDIMHFLNWYQQKPDGTVKLLIYYTSRLHSGVPSRFSGSGSGTDYSLTITNLEPEDIATYYCQQHSNLPPTFGGGTKLEIK

SEQ ID NO: 36 - protein, 5 aa, synthetic construct
NYGMN

SEQ ID NO: 37 - protein, 17 aa, synthetic construct
LINTYTRGPTFADDFKG

SEQ ID NO: 38 - protein, 12 aa, synthetic construct
RGEYYGSSYVDY

SEQ ID NO: 39 - protein, 11 aa, synthetic construct
RASQDIMHFLN

SEQ ID NO: 40 - protein, 7 aa, synthetic construct
YTSRLHS

SEQ ID NO: 41 - protein, 9 aa, synthetic construct
QQHSNLPPT

SEQ ID NO: 42 - protein, 23 aa, synthetic construct
DIQMTQITSSLSASLGDRVTISC

SEQ ID NO: 43 - protein, 15 aa, synthetic construct
WYQQKPDGTVKLLIY

SEQ ID NO: 44 - protein, 32 aa, synthetic construct
GVPSRFSGSGSGTDYSLTITNLEPEDIATYYC

SEQ ID NO: 45 - protein, 10 aa, synthetic construct
FGGGTKLEIK

SEQ ID NO: 46 - protein, 30 aa, synthetic construct; identical to SEQ ID NO: 22

SEQ ID NO: 47 - protein, 14 aa, synthetic construct
WVKQAPGKGLKWMG

SEQ ID NO: 48 - protein, 32 aa, synthetic construct
RFAFSVETSANTAYLQINNLKTEDTATYFCAR

SEQ ID NO: 49 - protein, 11 aa, synthetic construct
WGQGTTLTVSS

SEQ ID NO: 50 - protein, 117 aa, synthetic construct
  VARIANT 24: note = A or G
  VARIANT 48: note = I or M
QIQLVQSGPELKKPGETVKISCKXSGYTFTNYGMNWVKQAPGKGLKWXGWINTYTGDPTYADDFKGRXAFSLETSASXXYLQIDNLKXEDTATYFCARRDIFAMDYWGQGTSVTVSS

SEQ ID NO: 51 - protein, 107 aa, synthetic construct
  VARIANT 44: note = V or I
  VARIANT 48: note = F or I
  VARIANT 92: note = V or I
DIQMTQTTSSLSASLGDRVTITCRASQDIMKYLHWYQQKPDGTXKLLXYYTSRLHSGVPSRFSGSGSGTDYSLTISNLEQEDIATYFCQQDXKYPWTFGGGTKLEIK

SEQ ID NO: 52 - protein, 9 aa, synthetic construct
  VARIANT 4: note = V or I
QQDXKYPWT

SEQ ID NO: 53 - protein, 30 aa, synthetic construct
  VARIANT 24: note = A or G
QIQLVQSGPELKKPGETVKISCKXSGYTFT

SEQ ID NO: 54 - protein, 14 aa, synthetic construct
  VARIANT 13: note = I or M
WVKQAPGKGLKWXG

SEQ ID NO: 55 - protein, 28 aa, synthetic construct
  VARIANT 2: note = F or I
  VARIANT 12: note = N or T
  VARIANT 13: note = G or A
  VARIANT 22: note = K or N
RXAFSLETSASXXYLQIDNLKXEDTATY

SEQ ID NO: 56 - protein, 15 aa, synthetic construct
  VARIANT 10: note = V or I
  VARIANT 14: note = F or I
WYQQKPDGTXKLLXY

SEQ ID NO: 57 - protein, 9 aa, synthetic construct
QQDIKYPWT

SEQ ID NO: 58 - protein, 117 aa, synthetic construct
QIQLVQSGPELKKPGETVKISCKGSGYTFTNYGMNWVKQAPGKGLKWMGWINTYTGDPTYADDFKGRIAFSLETSASTAYLQIDNLKNEDTATYFCARRDIFAMDYWGQGTSVTVSS

SEQ ID NO: 59 - protein, 107 aa, synthetic construct
DIQMTQTTSSLSASLGDRVTITCRASQDIMKYLHWYQQKPDGTVKLLIYYTSRLHSGVPSRFSGSGSGTDYSLTISNLEQEDIATYFCQQDIKYPWTFGGGTKLEIK

SEQ ID NO: 60 - protein, 117 aa, synthetic construct; identical to SEQ ID NO: 1

SEQ ID NO: 61 - protein, 107 aa, synthetic construct
DIQMTQTTSSLSASLGDRVTITCRASQDIMKYLHWYQQKPDGTIKLLFYYTSRLHSGVPSRFSGSGSGTDYSLTISNLEQEDIATYFCQQDVKYPWTFGGGTKLEIK

SEQ ID NO: 62 - protein, 402 aa, synthetic construct
MKWMVVVLVCLQLLEAAVVKVPLKKFKSIRETMKEKGLLGEFLRTHKYDPAWKYRFGDLSVTYEPMAYMDAAYFGEISIGTPPQNFLVLFDTGSSNLWVPSVYCQSQACTSHSRFNPSESSTYSTNGQTFSLQYGSGSLTGFFGYDTLTVQSIQVPNQEFGLSENEPGTNFVYAQFDGIMGLAYPALSVDEATTAMQGMVQEGALTSPVFSVYLSNQQGSSGGAVVFGGVDSSLYTGQIYWAPVTQELYWQIGIEEFLIGGQASGWCSEGCQAIVDTGTSLLTVPQQYMSALLQATGAQEDEYGQFLVNCNSIQNLPSLTFIINGVEFPLPPSSYILSNNGYCTVGVEPTYLSSQNGQPLWILGDVFLRSYYSVYDLGNNRVGFATAAAAGGGSHHHHHHHH

SEQ ID NO: 63 - protein, 402 aa, synthetic construct
MKWLLLLGLVALSECIMYKVPLIRKKSLRRTLSERGLLKDFLKKHNLNPARKYFPQWEAPTLVDEQPLENYLDMEYFGTIGIGTPAQDFTVVFDTGSSNLWVPSVYCSSLACTNHNRFNPEDSSTYQSTSETVSITYGTGSMTGILGYDTVQVGGISDTNQIFGLSETEPGSFLYYAPFDGILGLAYPSISSSGATPVFDNIWNQGLVSQDLFSVYLSADDKSGSVVIFGGIDSSYYTGSLNWVPVTVEGYWQITVDSITMNGETIACAEGCQAIVDTGTSLLTGPTSPIANIQSDIGASENSDGDMVVSCSAISSLPDIVFTINGVQYPVPPSAYILQSEGSCISGFQGMNVPTESGELWILGDVFIRQYFTVFDRANNQVGLAPVAAAGGGSHHHHHHHH

SEQ ID NO: 64 - protein, 394 aa, Homo sapiens
MKWLLLLGLVALSECIMYKVPLIRKKSLRRTLSERGLLKDFLKKHNLNPARKYFPQWEAPTLVDEQPLENYLDMEYFGTIGIGTPAQDFTVVFDTGSSNLWVPSVYCSSLACTNHNRFNPEDSSTYQSTSETVSITYGTGSMTGILGYDTVQVGGISDTNQIFGLSETEPGSFLYYAPFDGILGLAYPSISSSGATPVFDNIWNQGLVSQDLFSVYLSADDKSGSVVIFGGIDSSYYTGSLNWVPVTVEGYWQITVDSITMNGETIACAEGCQAIVDTGTSLLTGPTSPIANIQSDIGASENSDGDMVVSCSAISSLPDIVFTINGVQYPVPPSAYILQSEGSCISGFQGMNVPTESGELWILGDVFIRQYFTVFDRANNQVGLAPVAAAGGGS

(19) United States
(12) Patent Application Publication
(10) Pub. No.: US 2026/0365759 A1
(43) Pub. Date: Nov. 19, 2026

(54) HIGH AFFINITY ANTIBODIES SPECIFICALLY BINDING TO PEPSINOGEN C

(71) Applicant: Roche Diagnostics GmbH, Mannheim (DE)

(72) Inventors: Hartmut Duefel, Schlehdorf (DE); Alfred Michael Engel, Weilheim (DE); Lars Hillringhaus, Koenigsdorf (DE); Klaus Hirzel, Baierbrunn (DE); Ulrike Kurtkaya, Graefelfing (DE); Thomas Meier, Muenchen (DE); Alexandra Moraru, Muenchen (DE); Anna Christine Muth, Germering (DE); Michael Schraeml, Penzberg (DE)

(21) Appl. No.: 19/705,645

(22) Filed: Jun. 11, 2026

Related U.S. Application Data
(63) Continuation of application No. PCT/EP2024/085509, filed on Dec. 10, 2024.

(30) Foreign Application Priority Data
Dec. 13, 2023 (EP) 23216327.9

(51) Int. Cl. C07K 16/40; G01N 33/573

(52) U.S. Cl. CPC C07K 16/40; C12Y 304/23003; G01N 33/573; G01N 2333/96477

(57) ABSTRACT

The present invention provides novel antibodies specifically binding to Pepsinogen C and compositions and kits comprising such antibodies. Furthermore, provided are polynucleotides encoding such antibodies, host cells expressing said antibodies, methods of producing such antibodies and diagnostic methods using such antibodies.

### CROSS-REFERENCE TO RELATED APPLICATIONS

This application is a continuation of International PCT Application No. PCT/EP2024/085509 filed on Dec. 10, 2024, which claims priority to European Patent Application No. 23216327.9 filed on Dec. 13, 2023, the disclosures of which are hereby incorporated by reference in their entirety.

### SEQUENCE LISTING

This application incorporates by reference in its entirety the material in the ST.26 XML file titled ROCHE-304 Sequence Listing.xml, which was created on Jun. 11, 2026 and is 64,678 bytes.

The present invention provides novel antibodies directed to Pepsinogen C and compositions and kits comprising such antibodies, especially in combination. Furthermore, provided are polynucleotides encoding such monoclonal antibodies, host cells expressing said antibodies, methods of producing such antibodies and diagnostic methods using such antibodies.

### BACKGROUND OF THE INVENTION

Alteration of the gastric secretion level is related to the pathogenesis of various kinds of diseases originating from the upper gastrointestinal (GI) tract. Classically, individuals with hyperchlorhydria, especially those with Helicobacter pylori infection, are predisposed to the development of duodenal ulcers, whereas individuals with hypochlorhydria are at the risk of gastric cancer through the development of a premalignant condition, gastric atrophy (El-Omar et al. 1995, 1997) Thus, measurement of the gastric acid secretion level has some clinical implications in estimating the risk for various diseases in the upper GI tract. However, given that the procedure causes significant distress to the subjects and is time-consuming, direct measurement of the gastric acid secretion level has become less prevalent these days.

Pepsinogen (PG) I (A) is secreted exclusively by the fundic glands (Samloff 1971), and PG II (C) is secreted by the fundic glands, pyloric glands, and proximal duodenal mucosa (Samloff and Liebman 1973). Previous studies have shown that the serum PG I (A) level and/or I/II (A/C) ratio reflect the morphological (Samloff et al. 1982; Borch et al. 1989) and functional statuses (Samloff et al. 1975; Nakanome et al. 1983; Miki et al. 1987; Feldman et al. 1988) of the gastric mucosa. Many studies have reported a significant correlation between serum PG values and gastric acid secretion levels (Samloff et al. 1975; Nakanome et al. 1983; Miki et al. 1987; Haruma et al. 1993; Kinoshita et al. 1997; Feldman et al. 1988; Iijima et al. 2005; Derakhshan et al. 2006).

Although there are assays for the detection of PG A and C available on the market, there is still the need for improvement. Especially it would be highly desirable to have greater precision especially at low analyte concentration, to use smaller sample volumes and to have faster assay results.

Therefore, there is a great need to i.a. identify high affinity antibodies which are specifically binding to Pepsinogen C.

The above need is addressed by the present invention described herein.

### BRIEF DESCRIPTION OF THE FIGURES

The following figures are provided to aid the understanding of the present invention, the true scope of which is set forth in the appended claims. It is understood that modifications can be made in the procedures set forth without departing from the spirit of the invention.

FIG. 1: ELISA of supernatants from mouse hybridoma-cultivation. The ELISA-Screening was performed with supernatants from hybridoma-cultivations as shown in FIG. 1.
(1) 96/384-well MTPs, streptavidin coated
(2) recombinant PepC, biotinylated
(3) mAb samples (supernatants of hybridoma-cultivations)
(4) anti-mouse-IgG POD-conjugate

FIG. 2: SPR (primary cultures). Kinetic Screening with exemplary kinetic signatures of antibody/Pepsinogen interactions. Shown is the binding to 150 nM PGA and PGC at 37 °C. Overlaid normalized sensorgrams for antibody interactions with specificity for PGC (top sensorgram) and no cross reactivity with PGA (bottom sensorgram).

FIG. 3: Kinetic signatures for mAbs 27, 75, 123, 11, 22 and 46 binding to PGC at 37 °C. Shown are series with increasing concentration c=1.2-450 nM, dilution factor 3.
Interactions are overlaid with a Langmuir 1:1 binding model (grey), Rmax global, RI=0

FIG. 4: Kinetic parameters determined using Biacore for mABs 46, 22, 11, 27, 75 and 123.

FIG. 5: Alignment showing VLs and VRs of mABs 22, 27, 75, 123 and 11 with CDRs boxed.

FIG. 6: Determination of the Limit of Quantification. 10 human serum samples were measured over 5 runs in 2 days in 5 replicates on 1 analyzer. This gave an intermediate precision CV of ≤20%.

FIG. 7: Determination of the Limit of Quantification. 10 human serum samples were measured over 5 runs in 2 days in 5 replicates on 1 analyzer. This gave an intermediate precision CV of ≤20%. Plot of coefficient of variation vs. mean concentration.

### DETAILED DESCRIPTION OF THE INVENTION

In the following, the elements of the present invention will be described. These elements are listed with specific aspects and embodiments, however, it should be understood that they may be combined in any manner and in any number to create additional aspects and embodiments.

The word “comprise”, and variations such as “comprises” and “comprising”, will be understood to imply the inclusion of a stated integer or step or group of integers or steps but not the exclusion of any other integer or step or group of integers or steps.

As used in this specification and the appended claims, the singular forms “a”, “an”, and “the” include plural referents, unless the content clearly dictates otherwise.

Concentrations, levels, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “150 mg to 600 mg” should be interpreted to include not only the explicitly recited values of 150 mg to 600 mg, but to also include individual values and sub-ranges within the indicated range. Thus, included in this numerical range are individual values such as 150, 160, 170, 180, 190, 580, 590, 600 mg and sub-ranges such as from 150 to 200, 150 to 250, 250 to 300, 350 to 600, etc. This same principle applies to ranges reciting only one numerical value. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

In general, the methods described are in vitro methods that are performed using a sample that has already been obtained from the subject (i.e., the sample is provided for the method, and the steps taken to obtain the sample from the subject are not included as part of the method). The methods may therefore include the step of providing a biological fluid sample from a subject.

As used herein, “provide”, “obtain” or “obtaining” can be any means whereby one comes into possession of the sample by “direct” or “indirect” means. Directly obtaining a sample means performing a process (e.g., performing a physical method such as extraction) to obtain the sample. Indirectly obtaining a sample refers to receiving the sample from another party or source (e.g., a third party laboratory that directly acquired the sample).

As used herein “affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein. Specific illustrative and exemplary methods for measuring binding affinity are described in the following.

The terms “anti-Pepsinogen C antibody” and “an antibody that specifically binds to Pepsinogen C” refer to an antibody that is capable of specifically binding Pepsinogen C. In one aspect of the invention, the extent of binding of an anti-Pepsinogen C antibody to a non-Pepsinogen C protein (e.g. Pepsinogen A) is less than about 10% of the binding of the antibody to Pepsinogen C as measured, e.g., by surface plasmon resonance (SPR). In certain aspects, an antibody that binds to Pepsinogen C has a dissociation constant (KD) of ≤100 nM, ≤10 nM, ≤1 nM, ≤0.6 nM. An antibody is said to “specifically bind” to an antigen (e.g. Pepsinogen C) when the antibody has a KD of 100 nM or less.

The terms “antibody”, “antibodies”, and analogous terms as used herein relate to full immunoglobulin molecules and encompass naturally-occurring forms of antibodies (including but not limited to IgG, IgA, IgM, IgE) as well as recombinant antibody constructs including but not limited to single-chain antibodies, chimeric antibodies, humanized antibodies, antibody-fusion proteins, and multi-specific antibodies; as well as antigen binding fragments and derivatives of all of the foregoing. The terms “antibody”, “antibodies”, and analogous terms as used herein also refer to antigen binding fragments thereof, which may be referenced herein as antibody antigen binding fragment, and/or, simply antigen binding fragment. These terms refer to one or more fragments of an antibody that retain the ability to specifically bind to the target antigen, i.e. Pepsinogen C, as known in the art, including but not limited to antigen binding fragments comprising an Fv domain, i.e., paired heavy and light chain variable domains, such as Fab, Fab′, F(ab′)2, and Fv fragments as well as recombinant constructs such as single-chain Fv domains, known in the art as scFvs. The terms also includes antibody antigen binding fragments that comprise a single, unpaired heavy or light chain variable domain as known in the art that retains the ability to specifically and selectively bind antigen as defined herein, including but not limited to single domain antibodies (also referenced in the art as sdAbs, dAbs, and/or nanobodies) and VHH domains based on the heavy chains of camelids.

In certain embodiments the monoclonal antibody of the invention may be a full-immunoglobulin, Fab, Fab′, F(ab′)2, Fv or scFv.

Antibodies may be polyclonal or monoclonal. Preferably, the antibodies of the invention are monoclonal. The term “monoclonal” as used herein with reference to an antibody or antigen binding fragment thereof, refer to a population of antibody polypeptides or fragments thereof produced from a single B cell clone, which population contains only one species of an antigen binding site capable of immunoreacting with a particular epitope of an antigen. This is in contrast with “polyclonal” antibodies and compositions, which term(s) refer to a population of antibody polypeptides or antigen binding fragments that contain multiple species of antigen binding sites. Also included are modified forms of monoclonal antibodies of the invention such as humanized or chimeric versions thereof, as well as recombinant antibody constructs, such as antibody (or antigen binding fragment)-fusion proteins, wherein the antibody or antigen binding fragment comprises (an) additional domain(s), e.g. for the isolation and/or preparation of recombinant produced antibody/fragment/constructs.

The term “epitope” denotes the site on an antigen, either proteinaceous or non-proteinaceous, to which an anti-Pepsinogen C antibody binds. Epitopes can be formed both from contiguous amino acid stretches (linear epitope) or comprise non-contiguous amino acids (conformational epitope), e.g., coming in spatial proximity due to the folding of the antigen, i.e. by the tertiary folding of a proteinaceous antigen. An epitope comprises at least 3, at least 4, at least 5, at least 6, at least 7, or 8-μ0 amino acids in a unique spatial conformation.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6th ed., W.H. Freeman and Co., page 9 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35 (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 30-36 (L1), 46-55 (L2), 89-96 (L3), 30-35 (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

The numbering of amino acid residues of the VH and VL of the antibody of the invention is made according to Kabat if not specifically mentioned otherwise. A skilled person can convert the numbering according to Kabat into other nomenclatures such as Clothia, McCallum, etc.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The term “substitution”, “Exchange” or “mutant” as used herein in the context of amino acids refers to the replacement of an amino acid at a certain position and the introduction of one (or more) amino acid(s) at a different position is explicitly not encompassed by the term “substitution”. As noted, the present invention encompasses conservative or highly conservative amino acid substitutions as have been defined herein.

Amino acids are herein either spelled out or abbreviated using a 1-letter code or a three letter code.

In the context of the invention, it is referred to variants of sequences (for example CDRs modified by one amino acid substitution). These variants typically comprise amino acid substitutions. It is evident that the variant CDRs are functional variants, i.e. having amino acid sequences that may differ from the reference amino acid sequence but which differing sequence exhibits or maintains the same functional activity as the reference sequence in the context of the described heavy and/or light chain variable domain. Specifically, as used herein, the term same functional activity means that the antibody or antibody binding fragment of the invention comprising one or more variant CDRs will maintain the extraordinary high association rate constant ka and/or the affinity with respect to the binding to Pepsinogen C.

As used in the context of the invention, a “conservative amino acid substitution” means the substitution of an amino acid with another amino acid selected from its same physicochemical group, wherein the physicochemical groups of amino acids are
a) the nonpolar, hydrophobic amino acids consisting of Gly, Ala, Val, Leu, Ile, Phe, Tyr, Trp, and Met;
b) the polar, neutral amino acids consisting of Ser, Thr, Asn, and Gln;
c) the positively charged, basic amino acids consisting of Arg, Lys, and His, and
d) the negatively charged, acidic amino acids consisting of Asp and Glu
   wherein if Cys is to be conservatively substituted, it is substituted with Ser or Ala, and
   wherein if Pro is to be conservatively substituted it is substituted with Ala.

As used in the context of the invention, a “highly conservative amino acid substitution” means the following amino acid substitutions:
a) substitution of Ala with Val, Leu, Ile or Gly;
b) substitution of Arg with Lys;
c) substitution of Asn with Gln;
d) substitution of Asp with Glu;
e) substitution of Cys with Ser;
f) substitution of Gln with Asn;
g) substitution of Glu with Asp;
h) substitution of Gly with Ala;
i) substitution of His with Arg;
j) substitution of Ile with Leu, Val or Ala;
k) substitution of Leu with Ile, Val or Ala;
l) substitution of Lys with Arg;
m) substitution of Met with Leu, Ile or Val;
n) substitution of Phe with Tyr or Trp;
o) substitution of Pro with Ala;
p) substitution of Ser with Thr;
q) substitution of Thr with Ser;
r) substitution of Trp with Phe or Tyr;
s) substitution of Tyr with Phe or Trp; and
t) substitution of Val with Leu, Ile or Ala.

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

The term “nucleic acid molecule”, “nucleic acid sequence” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Nucleic acid molecules also encompass DNA and RNA molecules which are suitable as a vector for direct expression of an antibody of the invention in vitro and/or in vivo, e.g., in a host. Such DNA (e.g., cDNA) or RNA (e.g. mRNA) vectors can be unmodified or modified. In embodiments, the nucleic acids/nucleic acid sequences of the invention may be isolated. Thus, the nucleic acid molecules may, for example, be nucleic acid molecules that have been synthesized according to conventional protocols of organic chemistry, according to recombinant methods, or produced semi-synthetically, e.g. by combining chemical synthesis and recombinant methods. The person skilled in the art is familiar with the preparation and the use of such nucleic acid molecules.

“Percent (%) homologues” in connection with amino acid sequences of polypeptides/peptides and/or nucleic acid sequences or nucleic acid molecules describes the number of matches of identical amino acid or nucleic acid residues of two or more aligned sequences as compared to the number of residues making up the overall length of the compared sequences (or the overall compared portions thereof). Using an alignment of two or more sequences or subsequences, the percentage of residues that are the same may be determined when the (sub)sequences are compared and aligned for maximum correspondence over a window of comparison, or over a designated region as measured using a sequence comparison algorithm as known in the art, or when manually aligned and visually inspected. Non-limiting examples of algorithms for use in determining sequence identity include, for example, those based on the NCBI BLAST algorithm (Altschul et al., Nucleic Acids Res 25(1997), 3389-3402), CLUSTALW computer program (Thompson, Nucl. Acids Res. 2(1994), 4673-4680) or FASTA (Pearson and Lipman, Proc. Natl. Acad. Sci., 85(1988), 2444). Although the FASTA algorithm typically does not consider internal non-matching deletions or additions in sequences, i.e. gaps, in its calculation, this can be corrected manually to avoid an overestimation of the % sequence identity. CLUSTALW, however, does take sequence gaps into account in its identity calculations. Also available are the BLAST and BLAST 2.0 algorithms (Altschul et al., Nucl Acids Res., 25(1977), 3389).

Preferable, the term “as shown in” in connection with amino acid sequences and/or nucleic acid sequences or nucleic acid molecules can be replaced by “consisting of” or “consists of”.

The term “Pepsinogen C”, as used herein, refers to any native Pepsinogen C from any vertebrate source, including mammals such as primates (e.g., humans) and rodents (e.g., mice and rats), unless otherwise indicated. The term encompasses “full-length”, unprocessed Pepsinogen C as well as any form of Pepsinogen C that results from processing in the cell. The term also encompasses naturally occurring variants of Pepsinogen C, e.g., splice variants or allelic variants. The amino acid sequence of an exemplary Pepsinogen C is shown in SEQ ID NO: 17. Human Pepsinogen C is shown in Uniprot P20142 (1991-02-01 v1).

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The term “Pepsinogen A”, as used herein, refers to any native Pepsinogen A from any vertebrate source, including mammals such as primates (e.g., humans) and rodents (e.g., mice and rats), unless otherwise indicated. The term encompasses “full-length”, unprocessed Pepsinogen A as well as any form of Pepsinogen A that results from processing in the cell. The term also encompasses naturally occurring variants of Pepsinogen A, e.g., splice variants or allelic variants. The amino acid sequence of an exemplary Pepsinogen A is shown in SEQ ID NO: 64. Furthermore, the term “Pepsinogen A” also encompasses isoforms thereof, specifically variants comprising a E58K, K222Q, T265A or V353L amino acid substitution (amino acid positions referred to SEQ ID NO: 64). Human Pepsinogen A is shown in Uniprot P0DJD9 (2012-02-22 v1).

“Immunoassays” as used herein are well-established bioanalytical methods in which detection or quantitation of an analyte depends on the reaction of the analyte and at least one analyte-specific binding agent, thus forming an analyte:binding agent complex. In the context of the present invention, at least one of the at least one analyte specific binding agent is an antibody of the invention. The specific embodiment of a “sandwich” immunoassay can be used for analytes possessing more than one recognition epitopes. Thus, a sandwich assay requires at least two binding agents that attach to non-overlapping epitopes on the analyte. In a “heterogeneous sandwich immunoassay” one of the binding agents has the functional role of an analyte-specific capture binding agent; this binding agent is or (during the course of the assay) becomes immobilized on a solid phase. A second analyte-specific binding agent is supplied in dissolved form in the liquid phase. A sandwich-like complex is formed once the respective analyte is bound by a first and a second binding agent (binding agent-1:analyte:binding agent-2). The sandwich-like complex is also referred to as “detection complex”. Within the detection complex the analyte is sandwiched between the binding agents, i.e. in such a complex the analyte represents a connecting element between the first binding agent and a second binding agent.

The term “heterogeneous” (as opposed to “homogeneous”) denotes two essential and separate steps in the assay procedure. In the first step, a detection complex containing label is formed and immobilized, however with unbound label still surrounding the complexes. Prior to determination of a label-dependent signal unbound label is removed from the immobilized detection complex, thus representing the second step. In contrast, a homogeneous assay produces an analyte-dependent detectable signal by way of single-step incubation and does not require a washing step.

In a heterogeneous immunoassay the solid phase is functionalized such that it may have bound to its surface the functional capture binding agent (the first binding agent), prior to being contacted with the analyte; or the surface of the solid phase is functionalized in order to be capable of anchoring a first binding agent, after it has reacted with the analyte. In the latter case, the anchoring process must not interfere with the binding agent's ability to specifically capture and bind the analyte. A second binding agent present in the liquid phase is used for detection of bound analyte. Thus, in a heterogeneous immunoassay the analyte is allowed to bind to the first (capture) and second (detector) binding agents. Thereby a “detection complex” is formed wherein the analyte is sandwiched between the capture binding agent and the detector binding agent. In a typical embodiment, the detector binding agent is labeled prior to being contacted with the analyte; alternatively a label is specifically attached to the detector binding agent after analyte binding. With the detection complexes being immobilized on the solid phase, the amount of label detectable on the solid phase corresponds to the amount of sandwiched analyte. After removal of unbound label, immobilized label indicating presence and amount of analyte can be detected.

“Detectable labels” as used herein relates to labels that allow for detection. According to an embodiment of the current invention a detectable label is an enzyme, or a label emitting light, in an embodiment fluorescence, luminescence, chemiluminescence, electrochemiluminescence or radioactivity. In a preferred embodiment the label is an electro-chemiluminescent label, in an embodiment Tris(2,2′-bipyridyl)ruthenium(II)-complex (Ru(bpy)). As the interference is caused by the three-dimensional structure of the label molecule that attracts auto-antibodies and similar interfering molecules and not by the signal-emitting mechanism of said label, such as e.g. light or radioactivity, all the above-referenced labels can be used in the current invention.

“Capture labels” as used herein relates to labels that can immobilize a capture agent (e.g. anti-Pepsinogen C antibody having a capture label attached thereto) on a surface (e.g., on a magnetic particle such as a microbead). Non-limiting examples are members of binding pairs. A non-limiting example for a capture label is biotin or derivatives thereof, which can interact with streptavidin or derivatives thereof. Different capture labels are well known in the art.

The methods provided herein comprise providing a biological fluid sample (for example a blood sample) from a subject. The samples being tested in the methods described herein are also referred to as “test samples” or “sample”.

As used herein, the terms “biological (fluid) sample”, “test sample”, “sample” are used interchangeably, and variations thereof refer to a sample obtained or derived from a subject. For the purposes described herein, the sample is, or comprises, a biological fluid (also referred to herein as a bodily fluid) sample.

A “sample” as used in the context of the present disclosure may be a liquid sample comprising or expected to comprise Pepsinogen C. The sample may in particular be a body fluid, such as, but not restricted to a blood sample, cerebrospinal fluid, seminal fluid, saliva or urine. In embodiments the blood sample can be venous blood or capillary blood. In embodiments, the sample is a blood sample, such as whole blood, serum or plasma. In embodiments, the sample is serum or plasma.

The sample is an in vitro sample, it will be analyzed in vitro and not transferred back into the body.

A blood sample may be a whole blood sample, or a processed blood sample e.g., serum, plasma etc. Methods for obtaining biological fluid samples (e.g., whole blood, serum, plasma, etc) from a subject are well known in the art. For example, methods for obtaining blood samples from a subject are well known and include established techniques used in phlebotomy. The obtained blood samples may be further processed using standard techniques to obtain e.g., a serum sample, or a plasma sample. Advantageously, methods for obtaining biological fluid samples from a subject are typically low-invasive or non-invasive.

A whole blood sample is defined as a blood sample drawn from the body and from which (substantially) no constituents (such as platelets or plasma) have been removed. In other words, the relative ratio of constituents in a whole blood sample is substantially the same as a blood in the body. In this context, “substantially the same” allows for a very small change in the relative ratio of the constituents of whole blood e.g., a change of up to 5%, up to 4%, up to 3%, up to 2%, up to 1% etc. Whole blood contains both the cell and fluid portions of blood. A whole blood sample may therefore also be defined as a blood sample with (substantially) all of its cellular components in plasma, wherein the cellular components (i.e., at least comprising the requisite white blood cells, red blood cells, platelets of blood) are intact.

The subject may be referred to herein as a patient. The terms “subject”, “individual”, and “patient” are used herein interchangeably and refer to an animal, preferably a mammal and, more typically to a human.

In the following detailed description of the invention, a number of individual elements, characterizing features, techniques and/or steps are disclosed. It is readily recognized that each of these has benefit not only individually when considered or used alone, but also when considered and used in combination with one another. Accordingly, to avoid exceedingly repetitious and redundant passages, this description has refrained from reiterating every possible combination and permutation. Nevertheless, whether expressly recited or not, it is understood that such combinations are entirely within the scope of the presently disclosed subject matter.

All technical and scientific terms used herein, unless otherwise defined, are intended to have the same meaning as commonly understood by one of ordinary skill in the art. Reference to techniques employed herein are intended to refer to the techniques as commonly understood in the art, including variations on those techniques or substitutions of equivalent techniques that would be apparent to one of skill in the art.

All amino acid sequences provided herein are presented starting with the most N-terminal residue and ending with the most C-terminal residue, as customarily done in the art, and the one-letter or three-letter code abbreviations as used to identify amino acids throughout the present invention correspond to those commonly used for amino acids.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

In an first aspect the invention relates to an antibody or antigen binding fragment thereof that specifically binds to Pepsinogen C and competes for binding to Pepsinogen C with the antibody consisting of (i) a heavy chain variable domain (VH) as shown in SEQ ID NO: 1; and (ii) a light chain variable domain (VL) as shown in SEQ ID NO: 2.

Pepsinogen (PG) C (PG II) is secreted by the fundig glands, pyloric glands, and procimal duodenal mucosa (Samloff and Liebman 1973). Previous studies have shown that the serum PG I level and/or I/II ratio reflect the morphological (Samloff et al. 1982; Borch et al. 1989) and functional statuses (Samloff et al. 1975; Nakanome et al. 1983; Miki et al. 1987; Feldman et al. 1988) of the gastric mucosa. Many studies have reported a significant correlation between serum PG values and gastric acid secretion levels. Alteration of the gastric secretion level is related to the pathogenesis of various kinds of diseases originating from the upper gastrointestinal (GI) tract. Classically, individuals with hyperchlorhydria, especially those with Helicobacter pylori infection, are predisposed to the development of duodenal ulcers, whereas individuals with hypochlorhydria are at the risk of gastric cancer through the development of a premalignant condition, gastric atrophy (El-Omar et al. 1995, 1997) Thus, measurement of the gastric acid secretion level has some clinical implications in estimating the risk for various diseases in the upper GI tract. However, given that the procedure causes significant distress to the subjects and is time-consuming, direct measurement of the gastric acid secretion level has become less prevalent these days. Therefore, antibodies specifically binding to Pepsinogen C which is a useful indicator for the measurement of the gastric acid secretion level and for estimating the risk for various disease in the upper GI tract are needed.

The inventors of the present invention have identified high affinity antibodies specifically binding to Pepsinogen C and competing for binding to Pepsinogen C with the antibody consisting of (i) a heavy chain variable domain (VH) as shown in SEQ ID NO: 1; and (ii) a light chain variable domain (VL) as shown in SEQ ID NO: 2.

An antibody that competes for binding to Pepsinogen C with an reference anti-Pepsinogen C antibody refers to an antibody that blocks binding of the reference anti-Pepsinogen C antibody to its antigen in a competition assay by 50% or more, and conversely, the reference antibody blocks binding of the antibody to its antigen in a competition assay by 50% or more. In embodiments antibodies compete for binding to e.g. Pepsinogen C when the antibodies bind to the same epitope of Pepsinogen C. Competitive binding can be used to determine whether an antibody binds to the same epitope of Pepsinogen C as, or competes for binding with, a reference anti-Pepsinogen C antibody. For example, an “antibody that binds to the same epitope” as a reference anti-Pepsinogen C antibody refers to an antibody that blocks binding of the reference anti-Pepsinogen C antibody to its antigen in a competition assay by 50% or more, and conversely, the reference antibody blocks binding of the antibody to its antigen in a competition assay by 50% or more. Also for example, to determine if an antibody binds to the same epitope as a reference anti-Pepsinogen C antibody, the reference antibody is allowed to bind to Pepsinogen C under saturating conditions. After removal of the excess of the reference anti-Pepsinogen C antibody, the ability of an anti-Pepsinogen C antibody in question to bind to Pepsinogen C is assessed. If the anti-Pepsinogen C antibody is able to bind to Pepsinogen C after saturation binding of the reference anti-Pepsinogen C antibody, it can be concluded that the anti-Pepsinogen C antibody in question binds to a different epitope than the reference anti-Pepsinogen C antibody. But, if the anti-Pepsinogen C antibody in question is not able to bind to Pepsinogen C after saturation binding of the reference anti-Pepsinogen C antibody, then the anti-Pepsinogen C antibody in question may bind to the same epitope as the epitope bound by the reference anti-Pepsinogen C antibody. To confirm whether the antibody in question binds to the same epitope or is just hampered from binding by steric reasons, and thus competes for binding with the reference anti-Pepsinogen C antibody without binding to the same epitope, routine experimentation can be used (e.g., peptide mutation and binding analyses using ELISA, RIA, surface plasmon resonance, flow cytometry or any other quantitative or qualitative antibody-binding assay available in the art). This assay should be carried out in two set-ups, i.e. with both of the antibodies being the saturating antibody. If, in both set-ups, only the first (saturating) antibody is capable of binding to Pepsinogen C, then it can be concluded that the anti-Pepsinogen C antibody in question and the reference anti-Pepsinogen C antibody compete for binding to Pepsinogen C.

In some aspects, two antibodies are deemed to bind to the same or an overlapping epitope if a 1-, 5-, 10-, 20- or 100-fold excess of one antibody inhibits binding of the other by at least 50%, at least 75%, at least 90% or even 99% or more as measured in a competitive binding assay (see, e.g., Junghans et al., Cancer Res. 50 (1990) 1495-1502).

To generate structural antibodies against Pepsinogen C, mice can be immunized with recombinant Pepsinogen C. After immunization, spleens of the immunized animals can be prepared and fused to myeloma-cells.

These hybridoma-cells can be cultivated and supernatants can be tested by ELISA for their reactivity against recombinant Pepsinogen C.

Selected Hybridoma clones can be taken into culture in a small scale dialysis-fermentation unit, and grown for production of IgG for several weeks with repeated change of dialysis media. The cell culture supernatant can be harvested, and IgG could be purified using standard affinity chromatography methods.

These antibodies can be screened for competing for binding for Pepsinogen C. This screening could be done as described above (e.g. using a competition assay or competitive binding assay as described above) or with a sandwich immunological assay using an automated Elecsys Immuno-Analyzer (Elecsys® Cobas® e411). For the sandwich immunological assay two sets of antibody-conjugates can be prepared. One set of antibodies may be labeled with one label (e.g. a Ruthenium label) and the second set of antibodies may be labeled with a different label (e.g. a Biotin label). Different combinations of antibodies labeled with different labels can be tested. Using this assay, antibodies not forming a sandwich can be identified and the competing binding can be identified based on the sandwich-compatibility (exemplary described in Example 8).

In one embodiment the antibody or antigen binding fragment thereof according to the invention comprises (a) a VH comprising a CDR-H1 as shown in the amino acid sequence of SEQ ID NO: 3 or a variant thereof modified by one amino acid substitution; a CDR-H2 as shown in the amino acid sequence of SEQ ID NO: 4, or a variant thereof modified by one amino acid substitution; and a CDR-H3 as shown in the amino acid sequence of SEQ ID NO: 5, or a variant thereof modified by one amino acid substitution; and a VL comprising a CDR-L1 as shown in the amino acid sequence of SEQ ID NO: 6 or a variant thereof modified by one amino acid substitution; a CDR-L2 as shown in the amino acid sequence of SEQ ID NO: 7, or a variant thereof modified by one amino acid substitution; and a CDR-L3 as shown in the amino acid sequence of SEQ ID NO: 52, or a variant thereof modified by one amino acid substitution.

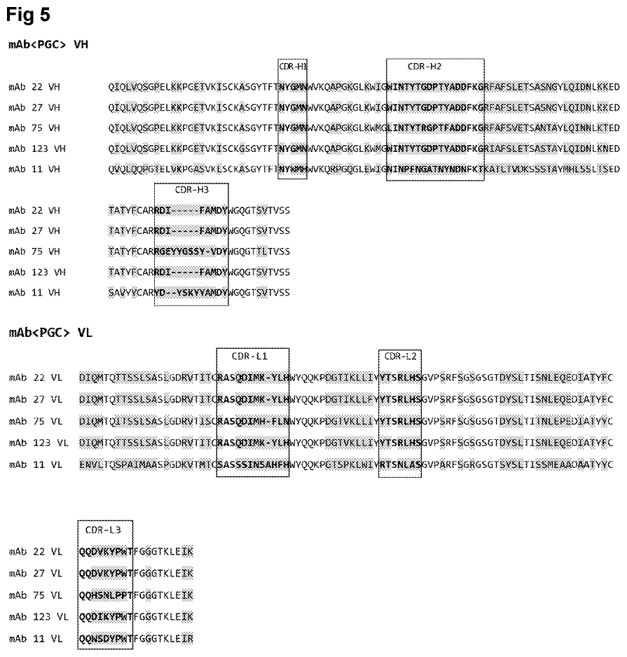

The present inventors surprisingly identified a family of antibodies which compete for specifically binding Pepsinogen C. As demonstrated in the appended Examples (e.g. Example 8, Table 1) the antibodies of this family (e.g. mAb 22, 27, 75 and 123) all compete for binding to Pepsinogen C. As can be seen in FIG. 5 the CDRs, which are i.a. responsible for the binding to the respective antigen, show very similar amino acid sequences. The CDR-L3 of three antibodies only show one amino acid variation, as shown in the consensus sequence SEQ ID NO: 52 (QQDXKYPWT, wherein X is the amino acid Val or Ile). Both are nonpolar, hydrophobic amino acids and show similar advantageous properties, such as the binding to the antigen.

In a further embodiment the antibody or antigen binding fragment thereof according to the invention comprises (b) a VH comprising a CDR-H1 as shown in the amino acid sequence of SEQ ID NO: 36 or a variant thereof modified by one amino acid substitution; a CDR-H2 as shown in the amino acid sequence of SEQ ID NO: 37, or a variant thereof modified by one amino acid substitution; and a CDR-H3 as shown in the amino acid sequence of SEQ ID NO: 38, or a variant thereof modified by one amino acid substitution; and a VL comprising a CDR-L1 as shown in the amino acid sequence of SEQ ID NO: 39 or a variant thereof modified by one amino acid substitution; a CDR-L2 as shown in the amino acid sequence of SEQ ID NO: 40, or a variant thereof modified by one amino acid substitution; and a CDR-L3 as shown in the amino acid sequence of SEQ ID NO: 41, or a variant thereof modified by one amino acid substitution.

In one embodiment all the amino acid substitutions are conservative amino acid substitutions. In a further embodiment each of said conservative amino acid substitutions is the substitution of an amino acid with another amino acid selected from its same group, wherein the groups of amino acids are
a) the nonpolar, hydrophobic amino acids consisting of Gly, Ala, Val, Leu, Ile, Phe, Try, Trp, and Met;
b) the polar, neutral amino acids consisting of Ser, Thr, Asn, and Gln;
c) the positively charged, basic amino acids consisting of Arg, Lys, and His, and
d) the negatively charged, amino acids consisting of Asp and Glu
wherein if Cys is to be conservatively substituted, it is substituted with Ser or Ala, and
wherein if Pro is to be conservatively substituted it is substituted with Ala.

In a further specific embodiment each of said conservative amino acid substitutions is a highly conservative amino acid substitution selected from
a) substitution of Ala with Val, Leu, Ile or Gly;
b) substitution of Arg with Lys;
c) substitution of Asn with Gln;
d) substitution of Asp with Glu;
e) substitution of Cys with Ser;
f) substitution of Gln with Asn;
g) substitution of Glu with Asp;
h) substitution of Gly with Ala,
i) substitution of His with Arg;
j) substitution of Ile with Leu, Val or Ala;
k) substitution of Leu with Ile, Val or Ala;
l) substitution of Lys with Arg;
m) substitution of Met with Leu, Ile or Val;
n) substitution of Phe with Try or Trp;
o) substitution of Pro with Ala;
p) substitution of Ser with Thr;
q) substitution of Thr with Ser;
r) substitution of Trp with Phe or Tyr;
s) substitution of Tyr with Phe or Trp; and
t) substitution of Val with Leu, Ile or Ala.

In a preferred embodiment the antibody or antigen binding fragment thereof according to invention comprises (a) a VH comprising a CDR-H1 as shown in the amino acid sequence of SEQ ID NO: 3; a CDR-H2 as shown in the amino acid sequence of SEQ ID NO: 4; and a CDR-H3 as shown in the amino acid sequence of SEQ ID NO: 5; and a VL comprising a CDR-L1 as shown in the amino acid sequence of SEQ ID NO: 6; a CDR-L2 as shown in the amino acid sequence of SEQ ID NO: 7; and a CDR-L3 as shown in the amino acid sequence of SEQ ID NO: 52. In a further embodiment the antibody or antigen binding fragment thereof comprises (b) a VH comprising a CDR-H1 as shown in the amino acid sequence of SEQ ID NO: 36; a CDR-H2 as shown in the amino acid sequence of SEQ ID NO: 37; and a CDR-H3 as shown in the amino acid sequence of SEQ ID NO: 38; and a VL comprising a CDR-L1 as shown in the amino acid sequence of SEQ ID NO: 39; a CDR-L2 as shown in the amino acid sequence of SEQ ID NO: 40; and a CDR-L3 as shown in the amino acid sequence of SEQ ID NO: 41.

In a more preferred embodiment the antibody or antigen binding fragment thereof according to the invention comprises (a) a VH comprising a CDR-H1 as shown in the amino acid sequence of SEQ ID NO: 3; a CDR-H2 as shown in the amino acid sequence of SEQ ID NO: 4; and a CDR-H3 as shown in the amino acid sequence of SEQ ID NO: 5; and a VL comprising a CDR-L1 as shown in the amino acid sequence of SEQ ID NO: 6; a CDR-L2 as shown in the amino acid sequence of SEQ ID NO: 7; and a CDR-L3 as shown in the amino acid sequence of SEQ ID NO: 8.

In a further embodiment the antibody or antigen binding fragment thereof according to the invention comprises (a) a VH comprising a CDR-H1 as shown in the amino acid sequence of SEQ ID NO: 3; a CDR-H2 as shown in the amino acid sequence of SEQ ID NO: 4; and a CDR-H3 as shown in the amino acid sequence of SEQ ID NO: 5; and a VL comprising a CDR-L1 as shown in the amino acid sequence of SEQ ID NO: 6; a CDR-L2 as shown in the amino acid sequence of SEQ ID NO: 7; and a CDR-L3 as shown in the amino acid sequence of SEQ ID NO: 57.

The antibody of the invention may have different framework regions flanking the CDRs in the VH and VL domain.

The light chain variable domain (VL) may consist of framework regions (FW) and CDRs as represented in formula I:

(FW-L1)-(CDR-L1)-(FW-L2)-(CDR-L2)-(FW-L3)-(CDR-L3)-(FW-L4) (formula I)
and the heavy chain variable domain (VH) may consist of FWs and CDRs as represented in formula II:

(FW-H1)-(CDR-H1)-(FW-H2)-(CDR-H2)-(FW-H3)-(CDR-H3)-(FW-H4) (formula II).

In one preferred embodiment the VH comprises a CDR-H1 as shown in the amino acid sequence of SEQ ID NO: 3; a CDR-H2 as shown in the amino acid sequence of SEQ ID NO: 4; and a CDR-H3 as shown in the amino acid sequence of SEQ ID NO: 5; and the VL comprises a CDR-L1 as shown in the amino acid sequence of SEQ ID NO: 6; a CDR-L2 as shown in the amino acid sequence of SEQ ID NO: 7; and a CDR-L3 as shown in the amino acid sequence of SEQ ID NO: 52 and the FWs comprise the following amino acid sequences or a variant thereof that is at least 85% homologous thereto:
FW-L1 the amino acid sequence of SEQ ID NO: 18;
FW-L2 the amino acid sequence of SEQ ID NO: 56;
FW-L3 the amino acid sequence of SEQ ID NO: 20;
FW-L4 the amino acid sequence of SEQ ID NO: 21;
FW-H1 the amino acid sequence of SEQ ID NO: 53;
FW-H2 the amino acid sequence of SEQ ID NO: 54;
FW-H3 the amino acid sequence of SEQ ID NO: 55;
FW-H4 the amino acid sequence of SEQ ID NO: 25.

The amino acid sequence of SEQ ID NO: 53 is QIQLVQSGPELKKPGETVKISCKX₁SGYTFT, the amino acid sequence of SEQ ID: 54 is WVKQAPGKGLKWX₂G, the amino acid sequence of SEQ ID NO: 55 is RX₃AFSLETSASX₄X₅YLQIDNLKX₆EDTATY and the amino acid sequence of SEQ ID NO: 56 is WYQQKPDGTX₇KLLX₃Y, wherein X₁ is Ala or Gly, X₂ is Ile or Met, X₃ is Phe or Ile, X₄ is Asn or Thr, X₅ is Gly or Ala, X₆ is Lys or Asn and X₇ is Val or Ile.

In another preferred embodiment the VH comprises a CDR-H1 as shown in the amino acid sequence of SEQ ID NO: 36; a CDR-H2 as shown in the amino acid sequence of SEQ ID NO: 37; and a CDR-H3 as shown in the amino acid sequence of SEQ ID NO: 38; and the VL comprises a CDR-L1 as shown in the amino acid sequence of SEQ ID NO: 39; a CDR-L2 as shown in the amino acid sequence of SEQ ID NO: 40; and a CDR-L3 as shown in the amino acid sequence of SEQ ID NO: 41 and the FWs comprise the following amino acid sequences or a variant thereof that is at least 85% homologous thereto:
FW-L1 the amino acid sequence of SEQ ID NO: 42;
FW-L2 the amino acid sequence of SEQ ID NO: 43;
FW-L3 the amino acid sequence of SEQ ID NO: 44;
FW-L4 the amino acid sequence of SEQ ID NO: 45;
FW-H1 the amino acid sequence of SEQ ID NO: 46;
FW-H2 the amino acid sequence of SEQ ID NO: 47;
FW-H3 the amino acid sequence of SEQ ID NO: 48;
FW-H4 the amino acid sequence of SEQ ID NO: 49.

In one preferred embodiment the VH comprises a CDR-H1 as shown in the amino acid sequence of SEQ ID NO: 3; a CDR-H2 as shown in the amino acid sequence of SEQ ID NO: 4; and a CDR-H3 as shown in the amino acid sequence of SEQ ID NO: 5; and the VL comprises a CDR-L1 as shown in the amino acid sequence of SEQ ID NO: 6; a CDR-L2 as shown in the amino acid sequence of SEQ ID NO: 7; and a CDR-L3 as shown in the amino acid sequence of SEQ ID NO: 52 and the FWs comprise the following amino acid sequences or a variant thereof modified by at most three amino acid substitutions:
FW-L1 the amino acid sequence of SEQ ID NO: 18;
FW-L2 the amino acid sequence of SEQ ID NO: 56;
FW-L3 the amino acid sequence of SEQ ID NO: 20;
FW-L4 the amino acid sequence of SEQ ID NO: 21;
FW-H1 the amino acid sequence of SEQ ID NO: 53;
FW-H2 the amino acid sequence of SEQ ID NO: 54;
FW-H3 the amino acid sequence of SEQ ID NO: 55;
FW-H4 the amino acid sequence of SEQ ID NO: 25.

In another preferred embodiment the VH comprises a CDR-H1 as shown in the amino acid sequence of SEQ ID NO: 36; a CDR-H2 as shown in the amino acid sequence of SEQ ID NO: 37; and a CDR-H3 as shown in the amino acid sequence of SEQ ID NO: 38; and the VL comprises a CDR-L1 as shown in the amino acid sequence of SEQ ID NO: 39; a CDR-L2 as shown in the amino acid sequence of SEQ ID NO: 40; and a CDR-L3 as shown in the amino acid sequence of SEQ ID NO: 41 and the FWs comprise the following amino acid sequences or a variant thereof modified by at most three amino acid substitutions:
FW-L1 the amino acid sequence of SEQ ID NO: 42;
FW-L2 the amino acid sequence of SEQ ID NO: 43;
FW-L3 the amino acid sequence of SEQ ID NO: 44;
FW-L4 the amino acid sequence of SEQ ID NO: 45;
FW-H1 the amino acid sequence of SEQ ID NO: 46;
FW-H2 the amino acid sequence of SEQ ID NO: 47;
FW-H3 the amino acid sequence of SEQ ID NO: 48;
FW-H4 the amino acid sequence of SEQ ID NO: 49.

In one embodiment all the amino acid substitutions are conservative amino acid substitutions. In a further embodiment each of said conservative amino acid substitutions is the substitution of an amino acid with another amino acid selected from its same group, wherein the groups of amino acids are
a) the nonpolar, hydrophobic amino acids consisting of Gly, Ala, Val, Leu, Ile, Phe, Try, Trp, and Met;
b) the polar, neutral amino acids consisting of Ser, Thr, Asn, and Gln;
c) the positively charged, basic amino acids consisting of Arg, Lys, and His, and
d) the negatively charged, amino acids consisting of Asp and Glu
wherein if Cys is to be conservatively substituted, it is substituted with Ser or Ala, and
wherein if Pro is to be conservatively substituted it is substituted with Ala.

In a further specific embodiment each of said conservative amino acid substitutions is a highly conservative amino acid substitution selected from
a) substitution of Ala with Val, Leu, Ile or Gly;
b) substitution of Arg with Lys;
c) substitution of Asn with Gln;
d) substitution of Asp with Glu;
e) substitution of Cys with Ser;
f) substitution of Gln with Asn;
g) substitution of Glu with Asp;
h) substitution of Gly with Ala;
i) substitution of His with Arg;
j) substitution of Ile with Leu, Val or Ala;
k) substitution of Leu with Ile, Val or Ala;
l) substitution of Lys with Arg;
m) substitution of Met with Leu, Ile or Val;
n) substitution of Phe with Try or Trp;
o) substitution of Pro with Ala;
p) substitution of Ser with Thr;
q) substitution of Thr with Ser;
r) substitution of Trp with Phe or Tyr;
s) substitution of Tyr with Phe or Trp; and
t) substitution of Val with Leu, Ile or Ala.

In one embodiment the antibody or antigen binding fragment thereof according to the invention comprises (i) a VH comprising a sequence as shown in SEQ ID NO: 1; and (ii) a VL comprising a sequence as shown in SEQ ID NO: 2.

In a further embodiment the antibody or antigen binding fragment thereof according to the invention comprises (i) a VH comprising a sequence as shown in SEQ ID NO: 1; or (ii) a VL comprising a sequence as shown in SEQ ID NO: 2.

In another embodiment the antibody or antigen binding fragment thereof according to the invention comprises (i) a VH comprising a sequence as shown in SEQ ID NO: 58; and (ii) a VL comprising a sequence as shown in SEQ ID NO: 59.

In a further embodiment the antibody or antigen binding fragment thereof according to the invention comprises (i) a VH comprising a sequence as shown in SEQ ID NO: 58; or (ii) a VL comprising a sequence as shown in SEQ ID NO: 59.

In one embodiment the antibody or antigen binding fragment thereof according to the invention comprises (i) a VH comprising a sequence as shown in SEQ ID NO: 60; and (ii) a VL comprising a sequence as shown in SEQ ID NO: 61.

In one embodiment the antibody or antigen binding fragment thereof according to the invention comprises (i) a VH comprising a sequence as shown in SEQ ID NO: 60; or (ii) a VL comprising a sequence as shown in SEQ ID NO: 61.

In another embodiment the antibody or antigen binding fragment thereof according to the invention comprises (i) a VH comprising a sequence as shown in SEQ ID NO: 34; and (ii) a VL comprising a sequence as shown in SEQ ID NO: 35.

In another embodiment the antibody or antigen binding fragment thereof according to the first aspect comprises (i) a VH comprising a sequence as shown in SEQ ID NO: 34; or (ii) a VL comprising a sequence as shown in SEQ ID NO: 35.

In a second aspect the invention relates to an antibody or antigen binding fragment thereof that specifically binds to Pepsinogen C and competes for binding to Pepsinogen C with the antibody consisting of
(i) a heavy chain variable domain (VH) as shown in SEQ ID NO: 9; and
(ii) a light chain variable domain (VL) as shown in SEQ ID NO: 10.

In one embodiment the antibody or antigen binding fragment thereof according to the invention comprises a VH comprising a CDR-H1 as shown in the amino acid sequence of SEQ ID NO: 11 or a variant thereof modified by one amino acid substitution; a CDR-H2 as shown in the amino acid sequence of SEQ ID NO: 12, or a variant thereof modified by one amino acid substitution; and a CDR-H3 as shown in the amino acid sequence of SEQ ID NO: 13, or a variant thereof modified by one amino acid substitution; and a VL comprising a CDR-L1 as shown in the amino acid sequence of SEQ ID NO: 14 or a variant thereof modified by one amino acid substitution; a CDR-L2 as shown in the amino acid sequence of SEQ ID NO: 15, or a variant thereof modified by one amino acid substitution; and a CDR-L3 as shown in the amino acid sequence of SEQ ID NO: 16, or a variant thereof modified by one amino acid substitution.

In one embodiment all the amino acid substitutions are conservative amino acid substitutions. In a further embodiment each of said conservative amino acid substitutions is the substitution of an amino acid with another amino acid selected from its same group, wherein the groups of amino acids are
a) the nonpolar, hydrophobic amino acids consisting of Gly, Ala, Val, Leu, Ile, Phe, Try, Trp, and Met;
b) the polar, neutral amino acids consisting of Ser, Thr, Asn, and Gln;
c) the positively charged, basic amino acids consisting of Arg, Lys, and His, and
d) the negatively charged, amino acids consisting of Asp and Glu
wherein if Cys is to be conservatively substituted, it is substituted with Ser or Ala, and
wherein if Pro is to be conservatively substituted it is substituted with Ala.

In a further specific embodiment each of said conservative amino acid substitutions is a highly conservative amino acid substitution selected from
a) substitution of Ala with Val, Leu, Ile or Gly;
b) substitution of Arg with Lys;
c) substitution of Asn with Gln;
d) substitution of Asp with Glu;
e) substitution of Cys with Ser;
f) substitution of Gln with Asn;
g) substitution of Glu with Asp;
h) substitution of Gly with Ala;
i) substitution of His with Arg;
j) substitution of Ile with Leu, Val or Ala;
k) substitution of Leu with Ile, Val or Ala;
l) substitution of Lys with Arg;
m) substitution of Met with Leu, Ile or Val;
n) substitution of Phe with Try or Trp;
o) substitution of Pro with Ala;
p) substitution of Ser with Thr;
q) substitution of Thr with Ser;
r) substitution of Trp with Phe or Tyr;
s) substitution of Tyr with Phe or Trp; and
t) substitution of Val with Leu, Ile or Ala.

In a further embodiment the antibody or antigen binding fragment thereof according to the invention comprises a VH comprising a CDR-H1 as shown in the amino acid sequence of SEQ ID NO: 11; a CDR-H2 as shown in the amino acid sequence of SEQ ID NO: 12; and a CDR-H3 as shown in the amino acid sequence of SEQ ID NO: 13; and a VL comprising a CDR-L1 as shown in the amino acid sequence of SEQ ID NO: 14; a CDR-L2 as shown in the amino acid sequence of SEQ ID NO: 15; and a CDR-L3 as shown in the amino acid sequence of SEQ ID NO: 16.

In a further embodiment the antibody or antigen binding fragment thereof according to the second aspect of the invention comprises a VL consisting of framework regions (FW) and CDRs as represented in formula I:

(FW-L1)-(CDR-L1)-(FW-L2)-(CDR-L2)-(FW-L3)-(CDR-L3)-(FW-L4) (formula I)
and a VH consisting of FWs and CDRs as represented in formula II:

(FW-H1)-(CDR-H1)-(FW-H2)-(CDR-H2)-(FW-H3)-(CDR-H3)-(FW-H4) (formula II),
wherein the VH comprises a CDR-H1 as shown in the amino acid sequence of SEQ ID NO: 11 or a variant thereof modified by one amino acid substitution; a CDR-H2 as shown in the amino acid sequence of SEQ ID NO: 12, or a variant thereof modified by one amino acid substitution; and a CDR-H3 as shown in the amino acid sequence of SEQ ID NO: 13, or a variant thereof modified by one amino acid substitution; and a VL comprising a CDR-L1 as shown in the amino acid sequence of SEQ ID NO: 14 or a variant thereof modified by one amino acid substitution; a CDR-L2 as shown in the amino acid sequence of SEQ ID NO: 15, or a variant thereof modified by one amino acid substitution; and a CDR-L3 as shown in the amino acid sequence of SEQ ID NO: 16, or a variant thereof modified by one amino acid substitution and the FWs comprise the following amino acid sequences or a variant thereof that is at least 85% homologous thereto:
FW-L1 the amino acid sequence of SEQ ID NO: 26;
FW-L2 the amino acid sequence of SEQ ID NO: 27;
FW-L3 the amino acid sequence of SEQ ID NO: 28;
FW-L4 the amino acid sequence of SEQ ID NO: 29;
FW-H1 the amino acid sequence of SEQ ID NO: 30;
FW-H2 the amino acid sequence of SEQ ID NO: 31;
FW-H3 the amino acid sequence of SEQ ID NO: 32;
FW-H4 the amino acid sequence of SEQ ID NO: 33.

In a further embodiment the antibody or antigen binding fragment thereof according to the second aspect of the invention comprises a VL consisting of framework regions (FW) and CDRs as represented in formula I:

(FW-L1)-(CDR-L1)-(FW-L2)-(CDR-L2)-(FW-L3)-(CDR-L3)-(FW-L4) (formula I)
and a VH consisting of FWs and CDRs as represented in formula II:

(FW-H1)-(CDR-H1)-(FW-H2)-(CDR-H2)-(FW-H3)-(CDR-H3)-(FW-H4) (formula II),
wherein the VH comprises a CDR-H1 as shown in the amino acid sequence of SEQ ID NO: 11 or a variant thereof modified by one amino acid substitution; a CDR-H2 as shown in the amino acid sequence of SEQ ID NO: 12, or a variant thereof modified by one amino acid substitution; and a CDR-H3 as shown in the amino acid sequence of SEQ ID NO: 13, or a variant thereof modified by one amino acid substitution; and a VL comprising a CDR-L1 as shown in the amino acid sequence of SEQ ID NO: 14 or a variant thereof modified by one amino acid substitution; a CDR-L2 as shown in the amino acid sequence of SEQ ID NO: 15, or a variant thereof modified by one amino acid substitution; and a CDR-L3 as shown in the amino acid sequence of SEQ ID NO: 16, or a variant thereof modified by one amino acid substitution and the FWs comprise the following amino acid sequences or a variant thereof modified by at most three amino acid substitutions:
FW-L1 the amino acid sequence of SEQ ID NO: 26;
FW-L2 the amino acid sequence of SEQ ID NO: 27;
FW-L3 the amino acid sequence of SEQ ID NO: 28;
FW-L4 the amino acid sequence of SEQ ID NO: 29;
FW-H1 the amino acid sequence of SEQ ID NO: 30;
FW-H2 the amino acid sequence of SEQ ID NO: 31;
FW-H3 the amino acid sequence of SEQ ID NO: 32;
FW-H4 the amino acid sequence of SEQ ID NO: 33.

In one embodiment all the amino acid substitutions are conservative amino acid substitutions. In a further embodiment each of said conservative amino acid substitutions is the substitution of an amino acid with another amino acid selected from its same group, wherein the groups of amino acids are
a) the nonpolar, hydrophobic amino acids consisting of Gly, Ala, Val, Leu, Ile, Phe, Try, Trp, and Met;
b) the polar, neutral amino acids consisting of Ser, Thr, Asn, and Gln;
c) the positively charged, basic amino acids consisting of Arg, Lys, and His, and
d) the negatively charged, amino acids consisting of Asp and Glu
wherein if Cys is to be conservatively substituted, it is substituted with Ser or Ala, and
wherein if Pro is to be conservatively substituted it is substituted with Ala.

In a further specific embodiment each of said conservative amino acid substitutions is a highly conservative amino acid substitution selected from
a) substitution of Ala with Val, Leu, Ile or Gly;
b) substitution of Arg with Lys;
c) substitution of Asn with Gln;
d) substitution of Asp with Glu;
e) substitution of Cys with Ser;
f) substitution of Gln with Asn;
g) substitution of Glu with Asp;
h) substitution of Gly with Ala;
i) substitution of His with Arg;
j) substitution of Ile with Leu, Val or Ala;
k) substitution of Leu with Ile, Val or Ala;
l) substitution of Lys with Arg;
m) substitution of Met with Leu, Ile or Val;
n) substitution of Phe with Try or Trp;
o) substitution of Pro with Ala;
p) substitution of Ser with Thr;
q) substitution of Thr with Ser;
r) substitution of Trp with Phe or Tyr;
s) substitution of Tyr with Phe or Trp; and
t) substitution of Val with Leu, Ile or Ala.

In a further embodiment the antibody or antigen binding fragment thereof according to the invention comprises (i) a VH comprising a sequence as shown in SEQ ID NO: 9; and (ii) a VL comprising a sequence as shown in SEQ ID NO: 10.

In a further embodiment the antibody or antigen binding fragment thereof according to the invention comprises (i) a VH comprising a sequence as shown in SEQ ID NO: 9; or (ii) a VL comprising a sequence as shown in SEQ ID NO: 10.

In a preferred embodiment the antibody or antigen binding fragment thereof according to the invention is a monoclonal antibody.

In a further embodiment of the invention, the antibody or antigen binding fragment thereof specifically binds to Pepsinogen C, wherein the Pepsinogen C comprises SEQ ID NO: 17. In one embodiment the Pepsinogen C consists of SEQ ID NO: 17.

In a further embodiment of the invention, the antibody or antigen binding fragment thereof does not specifically bind to Pepsinogen A. That means that the antibody or antigen binding fragment thereof binds to Pepsinogen A with a KD greater than 100 nM. In a preferred embodiment, the Pepsinogen A consists of SEQ ID NO: 64 or a variant thereof comprising a E58K, K222Q, T265A or V353L amino acid substitution.

As demonstrated in the appended examples (e.g. Examples 5 and 9), the antibody of the invention is also characterized by having a high affinity to Pepsinogen C consisting of SEQ ID NO: 62. Accordingly, the antibody of the invention may be characterized by having an equilibrium dissociation constant K_{D} of 2 nM or less, 1.5 nM or less, 1 nM or less, or 0.6 nM or less. In one preferred embodiment the K_{D} is measured at 37 °C. Decreasing K_{D} is preferred. The lower the K_{D}, the more preferred.

In a preferred embodiment, the K_{D} of the antibody or antigen binding fragment thereof according to the invention for the binding to Pepsinogen C is determined by surface plasmon resonance spectroscopy. The experimental settings are described herein in Example 9.

Methods for determining an equilibrium dissociation constant K_{D} are known in the art.

In a preferred embodiment, the K_{D} may be determined by surface plasmon resonance spectroscopy (e.g. BIAcore®).

The preferred temperature for determining the K_{D} is 37 °C.

In embodiments, the K_{D} may be determined using kinetic measurements determining the association and dissociation rate. Accordingly, the methods may in embodiments be the same surface plasmon resonance methods as described for determination of the kₐ, below. Said method may comprise correction for mass transport limitation.

In preferred embodiments, the K_{D} may be determined using surface plasmon resonance spectroscopy (e.g. BIAcore®).

In a further embodiment the antibody or antigen binding fragment thereof according to the invention has an antibody/antigen complex half-life t_{/2-diss} at 37 °C of 20 minutes or longer, preferably 30 minutes or longer.

Methods for determining the antibody/antigen complex half-life of an antibody for the binding to Pepsinogen C are known in the art. Exemplary examples are described in the Examples (in particular Example 9).

The antibody/antigen complex half-life was calculated in minutes according to the formula t_{/2 diss}=ln(2)/(k_{d}*60).

As demonstrated in the appended Examples, the antibody of the invention is characterized by a particularly high association rate constant (kₐ) for the binding to Pepsinogen C. This fast association rate makes the antibody of the invention particularly suitable for high throughput immunoassay and a competitive assay format.

Methods for determining the association rate constant (kₐ) of an antibody for the binding to Pepsinogen C are known in the art. Exemplary examples are described in the Examples (in particular Example 9).

Accordingly, in embodiments the antibody or antigen binding fragment thereof according to the invention is further characterized in that the association rate (kₐ) for the binding to Pepsinogen C comprising SEQ ID NO: 62 is at least 10⁵ M⁻¹s⁻¹, preferable at least 2.5×10⁵ M⁻¹s⁻¹. In one preferred embodiment, the association rate for the binding to Pepsinogen C comprising SEQ ID NO: 62 is at least 2.5×10⁵ M⁻¹s⁻¹. In one embodiment kₐ is measured at 37 °C. Decreasing K_{D} is preferred. The lower the K_{D}, the more preferred.

Methods for determining the association rate constant (kₐ) of an antibody for the binding to Pepsinogen C are known in the art. Exemplary examples are described in the Examples (in particular Example 9).

In a preferred embodiment, the association rate constant (kₐ) of the antibody or antigen binding fragment thereof according to the invention for the binding to Pepsinogen C is determined by surface plasmon resonance spectroscopy.

Said surface plasmon resonance spectroscopy comprises attaching or capturing the antibody, or antigen binding fragment thereof, on a CM5 sensor chip and injecting Pepsinogen C comprising SEQ ID NO: 17 as analyte, wherein said determination is conducted at a temperature of 37 °C using HBS-ET buffer (10 mM HEPES, 150 mM NaCl, 3 mM EDTA, 0.05% (w/v) Tween 20®).

In one embodiment the surface plasmon resonance spectroscopy is performed with a Biacore 8 k or B4000 instrument.

The association constant kₐ [1/Ms], the dissociation rate constant k_{d} [s⁻¹] and the dissociation equilibrium constant K_{D} [M] may be calculated according to a Langmuir fitting model, e.g. by using the evaluation software corresponding to the instrument.

The antibodies and antigen binding fragments of the invention may be prepared by a variety of techniques routinely used in the art. For example, antibodies can be prepared immunizing a non-human animal (e.g. mice) with Pepsinogen C isolating and subsequently isolating antigen-reactive, antibody producing B-cells. Preferred exemplary methods for the production of antibodies according to the invention using immunization of non-human animals are provided in the appended Examples (see in particular Example 3). Selected clones for producing antibodies can be processed according to routine methods for subsequent recombinant processing.

Another suitable method for producing or isolating antibodies and antibody antigen binding fragments of the invention include, but are not limited to, methods that select a recombinant antibody from a peptide or protein library (e.g., but not limited to, a bacteriophage, ribosome, oligonucleotide, RNA, cDNA, or yeast display library) using binding activities of interest. For example, antibodies or antigen binding fragments can be selected from such libraries by positively selecting for specific binding to Pepsinogen C. Display libraries are well known in the art and are, for example, available from various commercial vendors including but not limited to Cambridge Antibody Technologies (Cambridgeshire, UK), MorphoSys (Martinsried/Planegg, Del.), Biovation (Aberdeen, Scotland, UK) and Bioinvent (Lund, Sweden). Again, selected clones can be processed according to routine methods for subsequent recombinant processing.

Antibodies of the invention may be recombinantly expressed. Accordingly, in certain embodiments, the antibody of the invention may be a recombinant antibody. Methods for producing a recombinant antibody are known in the art.

In a third aspect the invention relates to a polynucleotide or a set of polynucleotides encoding
(i) the heavy chain variable domain of the antibody or antigen binding fragment thereof according to the invention, and
(ii) the light chain variable domain of the antibody or antigen binding fragment thereof according to the invention.

The present invention also provides a nucleic acid molecule encoding the antibody of the invention or any antigen-binding fragments thereof, as defined herein above. In particular provided is a polynucleotide encoding a heavy chain and/or light chain variable domain of the monoclonal antibody specifically binding to Pepsinogen C as defined herein above. In some embodiments, the polynucleotide may comprise further sequences to ensure that not only the heavy and/or light chain variable domain are expressed, but also the remaining heavy and/or light chain constant regions such that a full-length IgG antibody is expressed comprising the heavy and light chain variable domains of the invention. Accordingly, for each of the aspects and embodiments relating to antibodies or antigen binding fragments specifically binding Pepsinogen C as described herein a corresponding polynucleotide encoding the respective antibody or antigen binding fragment is provided herein.

In a fourth aspect of the invention, provided herein is a vector that comprises a polynucleotide of the invention. In particular, provided are vectors comprising a nucleic acid molecule encoding an antibody or antibody antigen binding fragment of the invention. As used herein, the term “vector” relates to a circular or linear nucleic acid molecule that can autonomously replicate in a host cell into which it has been introduced. Non-limiting examples of vectors suitable for use in the present invention include cosmids, plasmids (e.g., naked or contained in liposomes), viruses (e.g., lentiviruses, retroviruses, adenoviruses, and adeno-associated viruses) and bacteriophages. However, the art provides many suitable vectors, the choice of which depends on the desired function. The development and use of suitable vectors is well documented in the art; see, for example, the techniques described in Sambrook and Russel “Molecular Cloning, A Laboratory Manual”, Cold Spring Harbor Laboratory, N.Y. (2001) and Ausubel, “Current Protocols in Molecular Biology”, Green Publishing Associates and Wiley Interscience, N.Y. (1989), (1994). Vectors of use in connection with the present invention comprise a nucleic acid sequence encoding the full length Pepsinogen C antibody antigen binding fragment as disclosed herein. As such, for each of the aspects and embodiments relating to antibodies or antigen binding fragments specifically binding Pepsinogen C as described herein a vector comprising the corresponding polynucleotide encoding the respective antibody or antigen binding fragment is provided herein.

With regard to the term “vector comprising” as used herein, it is understood in the art that further nucleic acid sequences are present in the vectors that are necessary and/or sufficient for desired vector activity in the host cell, e.g. drive replication of the vector (and, thus the encoding nucleic acid sequences) and/or to direct the host cell express the antibody or antigen binding fragment of the invention. Such further nucleic acid sequences include but are not limited to sequences controlling vector replication and/or expression of a desired sequence in the particular cell system. For example, the vectors may comprise the nucleic acid molecule encoding an antibody or antibody antigen binding fragment of the invention operably linked and/or under the control of regulatory sequences. The term “regulatory sequence” refers to DNA sequences that are necessary to effect the expression of coding sequences to which they are operably linked. The term “control sequence” is intended to include, at a minimum, all components the presence of which may also be necessary for expression, and may further include additional advantageous components, e.g., to allow replication. As is understood in the art, the nature of such regulatory and control sequences differs depending upon the host organism. For example, in prokaryotes, control sequences generally include promoters, ribosomal binding sites, and terminators. In eukaryotes control sequences generally include promoters, terminators and, in some instances, enhancers, transactivators and/or transcription factors.

The vectors of use in the present invention are preferably expression vectors. An expression vector is capable of directing the replication and the expression of the nucleic acid molecule of the invention in a host cell and, accordingly, provides for the expression of, e.g., the heavy chain and/or light chain variable domains of the antibodies specifically binding to Pepsinogen C as disclosed herein. In some embodiments, the vector may comprise further sequences to ensure that not only the heavy and light chain variable domains are expressed, but also the remaining heavy and light chain constant regions such that a full-length IgG antibody is expressed comprising the heavy and light chain variable domains of the invention. Suitable expression vectors have been widely described in the literature and the determination of the appropriate expression vector for a particular cell system can be readily made by the skilled person using routine methods. Preferably, the vectors disclosed herein comprise a recombinant polynucleotide (i.e., a nucleic acid sequence encoding the monoclonal antibody according to the invention) as well as expression operably linked control sequences. The vectors as provided herein preferably further comprise a promoter. The herein described vectors may also comprise a selection marker gene and a replication-origin ensuring replication in the host Moreover, the herein provided vectors may also comprise a termination signal for transcription. Expression vectors as known in the art may drive transient or constitutive expression in a host cell.

The nucleic acid molecules and/or vectors of the invention can be designed for transfection into prokaryotic or eukaryotic host cells by any means known in the art or described herein. Non-limiting examples of suitable methods include chemical based methods (polyethylenimine, calcium phosphate, liposomes, DEAE-dextrane, nucleofection), nonchemical methods (electroporation, sonoporation, optical transfection, gene electrotransfer, hydrodynamic delivery or naturally occurring transformation upon contacting cells with the nucleic acid molecule of the invention), particle-based methods (gene gun, magnetofection, impalefection) phage vector-based methods and viral methods. For example, expression vectors derived from viruses such as retroviruses, vaccinia virus, adeno-associated virus, herpes viruses, Semliki Forest Virus or bovine papilloma virus, may be used for transfection of the nucleic acid molecules into targeted cell population. Additionally, baculoviral systems can also be used as vector in eukaryotic expression system for the nucleic acid molecules of the invention.

The term “prokaryote” is meant to include all bacteria which can be transformed, transduced or transfected with DNA or DNA or RNA molecules for the expression of a protein of the invention. Prokaryotic hosts may include gram negative as well as gram positive bacteria such as, for example, E. coli, S. typhimurium, Serratia marcescens, Corynebacterium (glutamicum), Pseudomonas (fluorescens), Lactobacillus, Streptomyces, Salmonella and Bacillus subtilis. The term “eukaryotic” is meant to include yeast, higher plant, insect and mammalian cells. Non-limiting examples of mammalian host cells typically used in the art include, Hela, HEK293, H9, Per.C6 and Jurkat cells, mouse NIH3T3, NS/0, SP2/0 and C127 cells, COS cells, e.g. COS 1 or COS 7, CV1, quail QC1-3 cells, mouse L cells, mouse sarcoma cells, Bowes melanoma cells and Chinese hamster ovary (CHO) cells.

Accordingly, in a fifth aspect the present invention relates to a host cell comprising a polynucleotide according to the invention, or a vector according to the invention. The host cell may be a prokaryotic cell or a eukaryotic cell. In a preferred embodiment, the host cell is a eukaryotic cell. In a particular embodiment, the cell is a HEK cell. In another particular embodiment the host cell is a CHO cell.

When recombinant expression vectors encoding the heavy and/or light chain of the antibody of the invention as disclosed herein are introduced into host cells, the antibodies or antibody antigen binding fragments are produced by culturing the host cells for a period of time sufficient to allow for expression of the antibody or antigen binding fragment in the host cell or, preferably, to allow for secretion of the antibody or antigen binding fragment into the culture medium in which the host cells are grown. Antibodies and/or antigen binding fragments can be recovered from the culture medium using standard protein purification methods. Methods for purification of antibodies are well known in the art. Exemplary purification methods are described in the appended Examples.

Accordingly, the invention also provides in a seventh aspect a method for the production of an antibody specifically binding to Pepsinogen C as disclosed herein. The method comprises culturing a host cell of the invention under suitable conditions and isolating the antibody produced. By purification steps, e.g. as described in the appended Examples an isolated antibody of the invention can be derived.

The invention further provides in an eighth aspect an antibody or an antigen binding fragment obtainable by any of the methods disclosed herein.

The transformed host cells can be grown in bioreactors and cultured according to techniques known in the art to achieve optimal cell growth. The antibody and/or antibody antigen binding fragment of the invention can then be isolated from the cell fraction or growth medium by any conventional means such, but not limited to, affinity chromatography (for example using a fusion-tag such as the Strep-tag II or the His₆ tag), gel filtration (size exclusion chromatography), anion exchange chromatography, cation exchange chromatography, hydrophobic interaction chromatography, high pressure liquid chromatography (HPLC), reversed phase HPLC or immunoprecipitation.

It will be appreciated that variations on the above procedures are within the scope of the present invention. For example, recombinant DNA technology may be used to remove or modify the DNA sequences encoding the antibodies and/or antibody antigen binding fragments disclosed herein, e.g. encoding the heavy and/or light chain variable domains as defined herein above. For example, recombinant DNA technology may be used to remove parts of the encoding sequence(s) that are not necessary for maintaining specific and selective binding to the antigen(s) of interest. The molecules expressed from such truncated DNA molecules are also encompassed by the antibodies of the invention.

Antibody derivatives can be produced, for example, by adding exogenous sequences to modify immunogenicity or reduce, enhance or modify binding, affinity, on-rate, off-rate, avidity, specificity, half-life, or any other suitable characteristic.

Also provided are humanized versions of the antibodies disclosed herein, i.e. comprising the CDRs of the heavy and or light chains as disclosed herein above. As well known in the art, “humanization” (to produce a humanized version of a parent antibody) refers to recombinantly engineering an antibody using CDRs derived from a non-human donor immunoglobulin in the context of human derived framework and constant domains. During the engineering, framework and/or CDR residues may be altered to preserve binding affinity and activity, e.g. specificity for Pepsinogen C. Methods to humanize antibodies are well known in the art, e.g. as disclosed in Queen et al., Proc. Natl. Acad Sci USA 86(1989), 10029-10032; Hodgson et al., Bio/Technology 9(1991) 421.

In a ninth aspect the invention provides a composition comprising an antibody of the invention, a polynucleotide of the invention, a vector of the invention, or a host cell of the invention. In a preferred embodiment, the composition is a diagnostic composition, i.e. a composition for use in diagnostic applications. In preferred embodiments, the composition is for use in an in vitro diagnostic test for detecting Pepsinogen C. In a preferred embodiment the diagnostic composition may be a reagent for an immunoassay for detecting Pepsinogen C. The diagnostic composition is preferably configured such that it allows for detection of Pepsinogen C in a sample obtained from a subject. The sample is preferably a blood sample (e.g. whole blood, serum or plasma).

In a tenth aspect the invention provides a composition comprising
a) the antibody or antigen binding fragment thereof according to the first aspect of the invention or according to any embodiment relating to the first aspect and
the antibody or antigen binding fragment thereof according the second aspect of the invention or according to any embodiment relating to the second aspect.

As demonstrated in Example 6 the antibodies of the invention were each labeled with one label. Two sets of antibody-conjugates were prepared. One set of antibodies were labeled with a Ruthenium label and the second set of antibodies were labeled with a Biotin label. In Example 7 different combinations of antibodies were tested. A Pepsinogen C containing sample was incubated with ruthenylated monoclonal Pepsinogen-C-specific IgG antibody and biotinylated monoclonal Pepsinogen-C-specific IgG antibody and thereby form a sandwich complex. As demonstrated in Table 1 the inventors surprisingly identified antibodies which in combination show a surprisingly high signal (e.g. antibodies like mAb 22, 27, 75 or 123 in combination with mAb 11 (i.e. antibodies binding the epitopes as the exemplary antibodies) compared to other combinations (e.g. mAb 46 combined with mAb 22, 27, 75 or 123). Without being bound by theory it seems that the epitopes bound by the antibodies influence which combinations of antibodies are advantageous (see Table 2).

Also provided is a composition comprising a polynucleotide, a vector or a host cell relating to the antibody of the first aspect of the invention or embodiments thereof and a polynucleotide, a vector or a host cell relating to the antibody of the second aspect of the invention or embodiments thereof.

In one embodiment the composition according to the invention is a diagnostic composition.

In particular the invention provides a composition comprising
the antibody or antigen binding fragment thereof according to the first aspect of the invention or to embodiments of the first aspect; and
the antibody or antigen binding fragment thereof according to the second aspect of the invention or to embodiments of the second aspect;
wherein the composition is a diagnostic composition.

In a eleventh aspect the invention relates to the use of the antibody or antigen binding fragment thereof according to the invention or the composition according to the invention for an in vitro immunoassay, in particular an in vitro immunoassay for detecting Pepsinogen C in a sample. Accordingly, also provided is an antibody of the invention for use in an immunoassay (e.g. a heterogeneous immunoassay) for detecting Pepsinogen C.

The sample may be a body fluid, such as, but not restricted to, a blood sample, cerebrospinal fluid, seminal fluid, saliva or urine. In embodiments, the sample is a blood sample, such as whole blood, serum or plasma. In embodiments, the sample is serum or plasma.

In a twelfth aspect, the present invention provides for an in vitro method for detecting Pepsinogen C in a sample using the antibody of the invention. The antibody may be part of a composition, in particular a diagnostic composition of the invention.

The sample may be a body fluid, such as a blood sample, cerebrospinal fluid, seminal fluid, saliva or urine. In embodiments, the sample is a blood sample, such as whole blood, serum or plasma. In embodiments, the sample is serum or plasma.

The method for detecting may comprise (a) contacting a sample comprising Pepsinogen C with the antibody, or antigen binding fragment thereof, according to the invention, thereby forming a complex comprising Pepsinogen C and said antibody, or antigen binding fragment thereof, and (b) detecting the complex formed in step (a), thereby detecting Pepsinogen C in said sample.

In one embodiment the Pepsinogen C in the sample comprises a sequence that is at least 95% homologous to the sequence as shown in SEQ ID NO: 17.

In one specific embodiment the method is a serum immunoassay, wherein the sample is a body fluid, preferably whole blood, serum or plasma.

In one specific embodiment the antibody, or antigen binding fragment thereof, has a detection label attached thereto.

The detection label linked to the antibody of the invention may be any detection label known in the art. For example, the detection label may selected from an enzyme, a label emitting light, preferable fluorescence, luminescence, chemiluminescence, electrochemiluminescence, or radioactivity.

In a thirteenth aspect the invention provides an in vitro method for detecting Pepsinogen C in a sample, comprising the following steps:
1) contacting a sample comprising Pepsinogen C with a first antibody, or antigen binding fragment thereof, and a second antibody, or antigen binding fragment thereof, wherein
(a) the first antibody is
(i) the antibody or antigen binding fragment thereof according to the first aspect of the invention or to embodiments of the first aspect; or
(ii) is the antibody or antigen binding fragment thereof according to the second aspect of the invention or to embodiments of the second aspect; and
(b) the second antibody specifically binds to Pepsinogen C and does not compete with the first antibody for specifically binding to Pepsinogen C;
2) incubating the resulting mixture of 1) to form a complex comprising Pepsinogen C and said first antibody or antigen binding fragment thereof and said second antibody or antigen binding fragment thereof, and
3) detecting Pepsinogen C in the sample by detecting the formation of the complex as defined in 2).

As already discussed above it is demonstrated in Example 6 that the antibodies of the invention were each labeled with one label. Two sets of antibodies were prepared. One set of antibodies were labeled with a Ruthenium label and the second set of antibodies were labeled with a Biotin label. In Example 7 different combinations of antibodies were tested. A Pepsinogen C containing sample was incubated with ruthenylated monoclonal Pepsinogen C-specific IgG antibody and biotinylated monoclonal Pepsinogen C-specific IgG antibody and thereby form a sandwich complex. As demonstrated in Table 1 the inventors surprisingly identified antibodies which in combination show a surprisingly high signal (e.g. antibodies like mAb22 or antibodies binding the same epitope (mAb27, 75 or 123) combined with mAb11) compared to other combinations (e.g. antibodies like mAb22 or antibodies binding the same epitope (mAb27, 75 or 123) combined with mAb46). Which combination of antibodies gives a particularly good signal was very surprising. Without being bound by theory it seems that the epitopes bound by the antibodies influence which combinations of antibodies are advantageous (see Table 2).

In one embodiment of the thirteenth aspect of the invention (c) one of the first and the second antibody comprises a detection label.

In a specific embodiment the first antibody or antigen binding fragment thereof or the second antibody or antigen binding fragment thereof not comprising the detection label, comprises a capture label.

“Capture labels” as used herein relates to labels that can immobilize a capture agent (e.g. an antibody according to the invention having a capture label attached thereto) on a surface (e.g., on a magnetic particle such as a microbead). Non-limiting examples are members of binding pairs. A non-limiting example for a capture label is biotin or derivatives thereof, which can interact with streptavidin or derivatives thereof. Different capture labels are well known in the art.

In one specific embodiment the method further comprises adding to the mixture of (1) particles capable of binding to the capture label and 2.1) separating the particles from the mixture before detecting a signal provided by the detection label.

In one embodiment
when the first antibody, or antigen binding fragment thereof, is selected from (i) than the second antibody is selected from (ii); or
when the first antibody, or antigen binding fragment thereof, is selected from (ii) than the second antibody is selected from (i).

In one embodiment the Pepsinogen C in the sample comprises a sequence that is at least 95% homologous to the sequence as shown in SEQ ID NO: 17.

In a specific embodiment the method is a serum immunoassay, wherein the sample is a body fluid, preferably whole blood, serum or plasma.

The detection label linked to the antibody of the invention may be any detection label known in the art. For example, the detection label may selected from an enzyme, a label emitting light, preferable fluorescence, luminescence, chemiluminescence, electrochemiluminescence, or radioactivity

In a further embodiment the Limit of Quantification (LoQ) of the method of the invention is 0.353 ng/mL at 20% coefficient of variation (CV).

The LoQ is defined as the lowest amount of analyte in a sample that can be accurately quantitated with an intermediate precision CV of ≤20%.

In a further embodiment the detection of Pepsinogen C in the sample is achieved in 18 minutes or less.

In a fourteenth aspect provided herein is a kit comprising the antibody of the invention or a composition comprising the same. In embodiments, the kit is a kit for detecting and/or quantifying Pepsinogen C in vitro. In embodiments, the kit is an immunoassay kit. In embodiments, the kit is a kit for a heterogenous immunoassay.

In a fifteenth aspect provided herein is a kit comprising the composition according to the tenth aspect of the invention, or a first antibody or antigen binding fragment thereof and a second antibody or antigen binding fragment thereof wherein one of the first or second antibody or antigen binding fragment thereof is the antibody of the first aspect of the invention or an embodiment of the first aspect and the other of the first or second antibody or antigen binding fragment thereof is the antibody of the second aspect of the invention or an embodiment of the second aspect.

As already discussed above it is demonstrated in Example 6 that the antibodies of the invention were each labeled with one label. Two sets of antibodies were prepared. One set of antibodies were labeled with a Ruthenium label and the second set of antibodies were labeled with a Biotin label. In Example 7 different combinations of antibodies were tested. A Pepsinogen C containing sample was incubated with ruthenylated monoclonal Pepsinogen C-specific IgG antibody and biotinylated monoclonal Pepsinogen C-specific IgG antibody and thereby form a sandwich complex. As demonstrated in Table 1 the inventors surprisingly identified antibodies which in combination show a surprisingly high signal (e.g. antibodies like mAb22 or antibodies binding the same epitope (mAb27, 75 or 123) combined with mAb11) compared to other combinations (e.g. antibodies like mAb22 or antibodies binding the same epitope (mAb27, 75 or 123) combined with mAb46). Which combination of antibodies gives a particularly good signal was very surprising. Without being bound by theory it seems that the epitopes bound by the antibodies influence which combinations of antibodies are advantageous (see Table 2).These advantageous combinations of antibodies can be comprised in the kit.

In one embodiment of the fifteenth aspect of the invention one of the first and the second antibody comprises a detection label.

In a specific embodiment the first antibody or antigen binding fragment thereof or the second antibody or antigen binding fragment thereof not comprising the detection label, comprises a capture label.

“Capture labels” as used herein relates to labels that can immobilize a capture agent (e.g. an antibody according to the invention having a capture label attached thereto) on a surface (e.g., on a magnetic particle such as a microbead). Non-limiting examples are members of binding pairs. A non-limiting example for a capture label is biotin or derivatives thereof, which can interact with streptavidin or derivatives thereof. Different capture labels are well known in the art.

In one specific embodiment the kit is immunoassay kit. In embodiments, the kit is a kit for an heterogeneous immunoassay.

In a sixteenth aspect the invention provides an antibody or antigen binding fragment thereof that specifically binds to Pepsinogen C, comprising
(i) a heavy chain variable domain (VH) that is at least 80% homologous to the VH as shown in SEQ ID NO: 50; and
(ii) a light chain variable domain (VL) that is at least 80% homologous to the VL as shown in SEQ ID NO: 51.

As demonstrated in Examples the inventors surprisingly identified a family of high affinity antibodies antibodies specifically binding to Pepsinogen C. As demonstrated in the appended Examples (e.g. Example 8, Table 1) several antibodies of this family (i.e. mAb 22, 27 and 123) all specifically bind to Pepsinogen C and share very similar amino acid sequences (FIG. 5). SEQ ID NO 50 and SEQ ID NO: 51 are the consensus sequences of the VH and VL, respectively.

In the amino acid sequence SEQ ID NO: 50 (QIQLVQSGPELKKPGETVKISCKX₁SGYTFTNYGMNWVKQAPGKGLKWX₂WIN TYTGDPTYADDFKGRX₃AFSLETSASX₄X₅YLQIDNLKX₆EDTATYFCARRDIFA MDYWGQGTSVTVSS) and SEQ ID NO: 51 (DIQMTQTTSSLSASLGDRVTITCRASQDIMKYLHWYQQKPDGTX₇KLLX₃YYSR LHSGVPSRFSGSGSGTDYSLTISNLEQEDIATYFCQQDX₇KYPWTFGGGTKLEIK) X₁ is Ala or Gly, X₂ is Ile or Met, X₃ is Phe or Ile, X₄ is Asn or Thr, X₅ is Gly or Ala, X₆ is Lys or Asn and X₇ is Val or Ile.

In one embodiment the antibody or antigen binding fragment thereof according to the invention comprises
(i) a VH that is at least 85% homologous to the VH as shown in SEQ ID NO: 50; and/or
(ii) a VL that is at least 85% homologous to the VL as shown in SEQ ID NO: 51.

In a further embodiment the antibody or antigen binding fragment thereof according to the invention comprises
(i) a VH that is at least 90% homologous to the VH as shown in SEQ ID NO: 50; and/or
(ii) a VL that is at least 90% homologous to the VL as shown in SEQ ID NO: 51.

In one embodiment the antibody or antigen binding fragment thereof according to the invention comprises
(i) a VH that is at least 95% homologous to the VH as shown in SEQ ID NO: 50; and/or
(ii) a VL that is at least 95% homologous to the VL as shown in SEQ ID NO: 51.

In one embodiment the antibody or antigen binding fragment thereof according to the invention comprises
(i) a VH that is at least 99% homologous to the VH as shown in SEQ ID NO: 50; and/or
(ii) a VL that is at least 99% homologous to the VL as shown in SEQ ID NO: 51.

In one embodiment the antibody or antigen binding fragment thereof according to the invention comprises
(i) a VH comprising a sequence as shown in SEQ ID NO: 50; and/or
(ii) a VL comprising a sequence as shown in SEQ ID NO: 51.

In one embodiment the antibody or antigen binding fragment thereof according to the invention comprises
(i) a VH consisting of the sequence as shown in SEQ ID NO: 50; and/or
(ii) a VL consisting of the sequence as shown in SEQ ID NO: 51.

In a seventeenth aspect the invention provides an antibody or antigen binding fragment thereof that specifically binds to Pepsinogen C, comprising
(i) a VH comprising a CDR-H1 as shown in the amino acid sequence of SEQ ID NO: 3 or a variant thereof modified by one amino acid substitution; a CDR-H2 as shown in the amino acid sequence of SEQ ID NO: 4, or a variant thereof modified by one amino acid substitution; and a CDR-H3 as shown in the amino acid sequence of SEQ ID NO: 5, or a variant thereof modified by one amino acid substitution; and
(ii) a VL comprising a CDR-L1 as shown in the amino acid sequence of SEQ ID NO: 6 or a variant thereof modified by one amino acid substitution; a CDR-L2 as shown in the amino acid sequence of SEQ ID NO: 7, or a variant thereof modified by one amino acid substitution; and a CDR-L3 as shown in the amino acid sequence of SEQ ID NO: 52, or a variant thereof modified by one amino acid substitution.

As can be seen in FIG. 5 the CDRs, which are i.a. responsible for the binding to the respective antigen, show very similar amino acid sequences. SEQ ID NO: 52 shows the consensus sequence of the CDR-L3 of the antibodies (QQDXKYPWT, wherein X is the amino acid Val or Ile).

In one embodiment the antibody or antigen binding fragment thereof according to the invention comprises
(i) a VH comprising a CDR-H1 as shown in the amino acid sequence of SEQ ID NO: 3; a CDR-H2 as shown in the amino acid sequence of SEQ ID NO: 4; and a CDR-H3 as shown in the amino acid sequence of SEQ ID NO: 5; and
(ii) a VL comprising a CDR-L1 as shown in the amino acid sequence of SEQ ID NO: 6; a CDR-L2 as shown in the amino acid sequence of SEQ ID NO: 7; and a CDR-L3 as shown in the amino acid sequence of SEQ ID NO: 52.

In one embodiment all the amino acid substitutions are conservative amino acid substitutions. In a further embodiment each of said conservative amino acid substitutions is the substitution of an amino acid with another amino acid selected from its same group, wherein the groups of amino acids are
a) the nonpolar, hydrophobic amino acids consisting of Gly, Ala, Val, Leu, Ile, Phe, Try, Trp, and Met;
b) the polar, neutral amino acids consisting of Ser, Thr, Asn, and Gln;
c) the positively charged, basic amino acids consisting of Arg, Lys, and His, and
d) the negatively charged, amino acids consisting of Asp and Glu
wherein if Cys is to be conservatively substituted, it is substituted with Ser or Ala, and
wherein if Pro is to be conservatively substituted it is substituted with Ala.

In a further specific embodiment each of said conservative amino acid substitutions is a highly conservative amino acid substitution selected from
a) substitution of Ala with Val, Leu, Ile or Gly;
b) substitution of Arg with Lys;
c) substitution of Asn with Gln;
d) substitution of Asp with Glu;
e) substitution of Cys with Ser;
f) substitution of Gln with Asn;
g) substitution of Glu with Asp;
h) substitution of Gly with Ala;
i) substitution of His with Arg;
j) substitution of Ile with Leu, Val or Ala;
k) substitution of Leu with Ile, Val or Ala;
l) substitution of Lys with Arg;
m) substitution of Met with Leu, Ile or Val;
n) substitution of Phe with Try or Trp;
o) substitution of Pro with Ala;
p) substitution of Ser with Thr;
q) substitution of Thr with Ser;
r) substitution of Trp with Phe or Tyr;
s) substitution of Tyr with Phe or Trp; and
t) substitution of Val with Leu, Ile or Ala.

In a further embodiment the antibody or antigen binding fragment thereof according to the seventeenth aspect of the invention comprises
a VL consisting of framework regions (FW) and CDRs as represented in formula I:

(FW-L1)-(CDR-L1)-(FW-L2)-(CDR-L2)-(FW-L3)-(CDR-L3)-(FW-L4) (formula I)
and a VH consisting of FWs and CDRs as represented in formula II:

(FW-H1)-(CDR-H1)-(FW-H2)-(CDR-H2)-(FW-H3)-(CDR-H3)-(FW-H4) (formula II),
wherein the FWs comprise the following amino acid sequences or a variant thereof that is at least 85% homologous thereto:
FW-L1 the amino acid sequence of SEQ ID NO: 18;
FW-L2 the amino acid sequence of SEQ ID NO: 56;
FW-L3 the amino acid sequence of SEQ ID NO: 20;
FW-L4 the amino acid sequence of SEQ ID NO: 21;
FW-H1 the amino acid sequence of SEQ ID NO: 53;
FW-H2 the amino acid sequence of SEQ ID NO: 54;
FW-H3 the amino acid sequence of SEQ ID NO: 55;
FW-H4 the amino acid sequence of SEQ ID NO: 25.

The amino acid sequence of SEQ ID NO: 53 is QIQLVQSGPELKKPGETVKISCKX₁SGYTFT, the amino acid sequence of SEQ ID: 54 is WVKQAPGKGLKWX₂G, the amino acid sequence of SEQ ID NO: 55 is RX₃AFSLETSASX₄X₅YLQIDNLKX₆EDTATY and the amino acid sequence of SEQ ID NO: 56 is WYQQKPDGTX₇KLLX₃Y, wherein X₁ is Ala or Gly, X₂ is Ile or Met, X₃ is Phe or Ile, X₄ is Asn or Thr, X₅ is Gly or Ala, X₆ is Lys or Asn and X₇ is Val or Ile.

In a further embodiment the antibody or antigen binding fragment thereof according to the seventeenth aspect of the invention comprises a VL consisting of framework regions (FW) and CDRs as represented in formula I:

(FW-L1)-(CDR-L1)-(FW-L2)-(CDR-L2)-(FW-L3)-(CDR-L3)-(FW-L4) (formula I)
and a VH consisting of FWs and CDRs as represented in formula II:

(FW-H1)-(CDR-H1)-(FW-H2)-(CDR-H2)-(FW-H3)-(CDR-H3)-(FW-H4) (formula II),
wherein the FWs comprise the following amino acid sequences or a variant thereof modified by at most three amino acid substitutions:
FW-L1 the amino acid sequence of SEQ ID NO: 18;
FW-L2 the amino acid sequence of SEQ ID NO: 56;
FW-L3 the amino acid sequence of SEQ ID NO: 20;
FW-L4 the amino acid sequence of SEQ ID NO: 21;
FW-H1 the amino acid sequence of SEQ ID NO: 53;
FW-H2 the amino acid sequence of SEQ ID NO: 54;
FW-H3 the amino acid sequence of SEQ ID NO: 55;
FW-H4 the amino acid sequence of SEQ ID NO: 25.

The amino acid sequence of SEQ ID NO: 53 is QIQLVQSGPELKKPGETVKISCKX₁SGYTFT, the amino acid sequence of SEQ ID: 54 is WVKQAPGKGLKWX₂G, the amino acid sequence of SEQ ID NO: 55 is RX₃AFSLETSASX₄X₅YLQIDNLKX₆EDTATY and the amino acid sequence of SEQ ID NO: 56 is WYQQKPDGTX₇KLLX₃Y, wherein X₁ is Ala or Gly, X₂ is Ile or Met, X₃ is Phe or Ile, X₄ is Asn or Thr, X₅ is Gly or Ala, X₆ is Lys or Asn and X₇ is Val or Ile.

In one embodiment all the amino acid substitutions are conservative amino acid substitutions. In a further embodiment each of said conservative amino acid substitutions is the substitution of an amino acid with another amino acid selected from its same group, wherein the groups of amino acids are
a) the nonpolar, hydrophobic amino acids consisting of Gly, Ala, Val, Leu, Ile, Phe, Try, Trp, and Met;
b) the polar, neutral amino acids consisting of Ser, Thr, Asn, and Gln;
c) the positively charged, basic amino acids consisting of Arg, Lys, and His, and
d) the negatively charged, amino acids consisting of Asp and Glu
wherein if Cys is to be conservatively substituted, it is substituted with Ser or Ala, and
wherein if Pro is to be conservatively substituted it is substituted with Ala.

In a further specific embodiment each of said conservative amino acid substitutions is a highly conservative amino acid substitution selected from
a) substitution of Ala with Val, Leu, Ile or Gly;
b) substitution of Arg with Lys;
c) substitution of Asn with Gln;
d) substitution of Asp with Glu;
e) substitution of Cys with Ser;
g) substitution of Gln with Asn;
g) substitution of Glu with Asp;
h) substitution of Gly with Ala;
i) substitution of His with Arg;
j) substitution of Ile with Leu, Val or Ala;
k) substitution of Leu with Ile, Val or Ala;
l) substitution of Lys with Arg;
m) substitution of Met with Leu, Ile or Val;
n) substitution of Phe with Try or Trp;
o) substitution of Pro with Ala;
p) substitution of Ser with Thr;
q) substitution of Thr with Ser;
r) substitution of Trp with Phe or Tyr;
s) substitution of Tyr with Phe or Trp; and
t) substitution of Val with Leu, Ile or Ala.

In one embodiment the antibody or antigen binding fragment thereof according to the invention comprises
(i) a VH comprising a sequence as shown in SEQ ID NO: 1; and
(ii) a VL comprising a sequence as shown in SEQ ID NO: 2.

In one embodiment the antibody or antigen binding fragment thereof according to the invention comprises
(i) a VH comprising a sequence as shown in SEQ ID NO: 1; or
(ii) a VL comprising a sequence as shown in SEQ ID NO: 2.

In another embodiment the antibody or antigen binding fragment thereof according to the invention comprises
(i) a VH comprising a sequence as shown in SEQ ID NO: 58; and
(ii) a VL comprising a sequence as shown in SEQ ID NO: 59.

In another embodiment the antibody or antigen binding fragment thereof according to the invention comprises
(i) a VH comprising a sequence as shown in SEQ ID NO: 58; or
(ii) a VL comprising a sequence as shown in SEQ ID NO: 59.

In a further the antibody or antigen binding fragment thereof according to the invention comprises
(i) a VH comprising a sequence as shown in SEQ ID NO: 60; and
(ii) a VL comprising a sequence as shown in SEQ ID NO: 61.

In a further embodiment the antibody or antigen binding fragment thereof according to the invention comprises
(i) a VH comprising a sequence as shown in SEQ ID NO: 60; or
(ii) a VL comprising a sequence as shown in SEQ ID NO: 61.

In a further embodiment of the sixteenth or seventeenth aspect or embodiments relating to the same the antibody or antigen binding fragment thereof binds to Pepsinogen C consisting of SEQ ID NO: 62 with a K_{D} of 0.1 nM or less, preferably wherein the K_{D} is measured at 37 °C.

In a further embodiment of the sixteenth or seventeenth aspect or embodiments relating to the same the antibody or antigen binding fragment thereof binds to Pepsinogen C consisting of SEQ ID NO: 62 with t/2-diss at 37 °C of 85 minutes or longer.

In a further embodiment of the sixteenth or seventeenth aspect or embodiments relating to the same the antibody or antigen binding fragment thereof has a association rate kₐ for Pepsinogen C consisting of SEQ ID NO: 62 of at least 0.9×10⁶ M⁻¹s⁻¹.

In an eighteenth aspect the invention relates to an antibody or antigen binding fragment thereof that specifically binds to Pepsinogen C, comprising
(i) a heavy chain variable domain (VH) that is at least 80% homologous to the VH as shown in SEQ ID NO: 34; and
(ii) a light chain variable domain (VL) that is at least 80% homologous to the VL as shown in SEQ ID NO: 35.

In one embodiment the antibody or antigen binding fragment thereof according to the invention comprises
(i) a VH that is at least 85% homologous to the VH as shown in SEQ ID NO: 34; and
(ii) a VL that is at least 85% homologous to the VL as shown in SEQ ID NO: 35.

In one embodiment the antibody or antigen binding fragment thereof according to the invention comprises
(i) a VH that is at least 85% homologous to the VH as shown in SEQ ID NO: 34; or
(ii) a VL that is at least 85% homologous to the VL as shown in SEQ ID NO: 35.

In one embodiment the antibody or antigen binding fragment thereof according to the invention comprises
(i) a VH that is at least 90% homologous to the VH as shown in SEQ ID NO: 34; and
(ii) a VL that is at least 90% homologous to the VL as shown in SEQ ID NO: 35.

In one embodiment the antibody or antigen binding fragment thereof according to the invention comprises
(i) a VH that is at least 90% homologous to the VH as shown in SEQ ID NO: 34; or
(ii) a VL that is at least 90% homologous to the VL as shown in SEQ ID NO: 35.

In one embodiment the antibody or antigen binding fragment thereof according to the invention comprises
(i) a VH that is at least 95% homologous to the VH as shown in SEQ ID NO: 34; and
(ii) a VL that is at least 95% homologous to the VL as shown in SEQ ID NO: 35.

In one embodiment the antibody or antigen binding fragment thereof according to the invention comprises
(i) a VH that is at least 95% homologous to the VH as shown in SEQ ID NO: 34; or
(ii) a VL that is at least 95% homologous to the VL as shown in SEQ ID NO: 35.

In one embodiment the antibody or antigen binding fragment thereof according to the invention comprises
(i) a VH that is at least 99% homologous to the VH as shown in SEQ ID NO: 34; and
(ii) a VL that is at least 99% homologous to the VL as shown in SEQ ID NO: 35.

In one embodiment the antibody or antigen binding fragment thereof according to the invention comprises
(i) a VH that is at least 99% homologous to the VH as shown in SEQ ID NO: 34; or
(ii) a VL that is at least 99% homologous to the VL as shown in SEQ ID NO: 35.

In one embodiment the antibody or antigen binding fragment thereof according to the invention comprises
(i) a VH comprising a sequence as shown in SEQ ID NO: 34; and
(ii) a VL comprising a sequence as shown in SEQ ID NO: 35.

In one embodiment the antibody or antigen binding fragment thereof according to the invention comprises
(i) a VH comprising a sequence as shown in SEQ ID NO: 34; or
(ii) a VL comprising a sequence as shown in SEQ ID NO: 35.

In one embodiment the antibody or antigen binding fragment thereof according to the invention comprises
(i) a VH consisting of the sequence as shown in SEQ ID NO: 34; and
(ii) a VL consisting of the sequence as shown in SEQ ID NO: 35.

In one embodiment the antibody or antigen binding fragment thereof according to the invention comprises
(i) a VH consisting of the sequence as shown in SEQ ID NO: 34; or
(ii) a VL consisting of the sequence as shown in SEQ ID NO: 35.

In one aspect the invention provides an antibody or antigen binding fragment thereof that specifically binds to Pepsinogen C, comprising
(i) a VH comprising a CDR-H1 as shown in the amino acid sequence of SEQ ID NO: 36 or a variant thereof modified by one amino acid substitution; a CDR-H2 as shown in the amino acid sequence of SEQ ID NO: 37, or a variant thereof modified by one amino acid substitution; and a CDR-H3 as shown in the amino acid sequence of SEQ ID NO: 38, or a variant thereof modified by one amino acid substitution; and
(ii) a VL comprising a CDR-L1 as shown in the amino acid sequence of SEQ ID NO: 39 or a variant thereof modified by one amino acid substitution; a CDR-L2 as shown in the amino acid sequence of SEQ ID NO: 40, or a variant thereof modified by one amino acid substitution; and a CDR-L3 as shown in the amino acid sequence of SEQ ID NO: 41, or a variant thereof modified by one amino acid substitution.

In one embodiment all the amino acid substitutions are conservative amino acid substitutions. In a further embodiment each of said conservative amino acid substitutions is the substitution of an amino acid with another amino acid selected from its same group, wherein the groups of amino acids are
a) the nonpolar, hydrophobic amino acids consisting of Gly, Ala, Val, Leu, Ile, Phe, Try, Trp, and Met;
b) the polar, neutral amino acids consisting of Ser, Thr, Asn, and Gln;
c) the positively charged, basic amino acids consisting of Arg, Lys, and His, and
d) the negatively charged, amino acids consisting of Asp and Glu
wherein if Cys is to be conservatively substituted, it is substituted with Ser or Ala, and
wherein if Pro is to be conservatively substituted it is substituted with Ala.

In a further specific embodiment each of said conservative amino acid substitutions is a highly conservative amino acid substitution selected from
a) substitution of Ala with Val, Leu, Ile or Gly;
b) substitution of Arg with Lys;
c) substitution of Asn with Gln;
d) substitution of Asp with Glu;
e) substitution of Cys with Ser;
f) substitution of Gln with Asn;
g) substitution of Glu with Asp;
h) substitution of Gly with Ala;
i) substitution of His with Arg;
j) substitution of Ile with Leu, Val or Ala;
k) substitution of Leu with Ile, Val or Ala;
l) substitution of Lys with Arg;
m) substitution of Met with Leu, Ile or Val;
n) substitution of Phe with Try or Trp;
o) substitution of Pro with Ala;
p) substitution of Ser with Thr;
q) substitution of Thr with Ser;
r) substitution of Trp with Phe or Tyr;
s) substitution of Tyr with Phe or Trp; and
t) substitution of Val with Leu, Ile or Ala.

In a further embodiment the antibody or antigen binding fragment thereof of the invention comprises (i) a VH comprising a CDR-H1 as shown in the amino acid sequence of SEQ ID NO: 36; a CDR-H2 as shown in the amino acid sequence of SEQ ID NO: 37; and a CDR-H3 as shown in the amino acid sequence of SEQ ID NO: 38; and (ii) a VL comprising a CDR-L1 as shown in the amino acid sequence of SEQ ID NO: 39; a CDR-L2 as shown in the amino acid sequence of SEQ ID NO: 40; and a CDR-L3 as shown in the amino acid sequence of SEQ ID NO: 41.

The antibody of the invention may have different framework regions flanking the CDRs in the VH and VL domain.

The light chain variable domain (VL) may consist of framework regions (FW) and CDRs as represented in formula I:

(FW-L1)-(CDR-L1)-(FW-L2)-(CDR-L2)-(FW-L3)-(CDR-L3)-(FW-L4) (formula I)
and the heavy chain variable domain (VH) may consist of FWs and CDRs as represented in formula II:

(FW-H1)-(CDR-H1)-(FW-H2)-(CDR-H2)-(FW-H3)-(CDR-H3)-(FW-H4) (formula II).

In one preferred embodiment the VH comprises a CDR-H1 as shown in the amino acid sequence of SEQ ID NO: 36; a CDR-H2 as shown in the amino acid sequence of SEQ ID NO: 37; and a CDR-H3 as shown in the amino acid sequence of SEQ ID NO: 38; and the VL comprises a CDR-L1 as shown in the amino acid sequence of SEQ ID NO: 39; a CDR-L2 as shown in the amino acid sequence of SEQ ID NO: 40; and a CDR-L3 as shown in the amino acid sequence of SEQ ID NO: 41 and the FWs comprise the following amino acid sequences or a variant thereof that is at least 85% homologous thereto:
FW-L1 the amino acid sequence of SEQ ID NO: 42;
FW-L2 the amino acid sequence of SEQ ID NO: 43;
FW-L3 the amino acid sequence of SEQ ID NO: 44;
FW-L4 the amino acid sequence of SEQ ID NO: 45;
FW-H1 the amino acid sequence of SEQ ID NO: 46;
FW-H2 the amino acid sequence of SEQ ID NO: 47;
FW-H3 the amino acid sequence of SEQ ID NO: 48;
FW-H4 the amino acid sequence of SEQ ID NO: 49.

In one preferred embodiment the VH comprises a CDR-H1 as shown in the amino acid sequence of SEQ ID NO: 36; a CDR-H2 as shown in the amino acid sequence of SEQ ID NO: 37; and a CDR-H3 as shown in the amino acid sequence of SEQ ID NO: 38; and the VL comprises a CDR-L1 as shown in the amino acid sequence of SEQ ID NO: 39; a CDR-L2 as shown in the amino acid sequence of SEQ ID NO: 40; and a CDR-L3 as shown in the amino acid sequence of SEQ ID NO: 41 and the FWs comprises the following amino acid sequences or a variant thereof modified by at most three amino acid substitutions:
FW-L1 the amino acid sequence of SEQ ID NO: 42;
FW-L2 the amino acid sequence of SEQ ID NO: 43;
FW-L3 the amino acid sequence of SEQ ID NO: 44;
FW-L4 the amino acid sequence of SEQ ID NO: 45;
FW-H1 the amino acid sequence of SEQ ID NO: 46;
FW-H2 the amino acid sequence of SEQ ID NO: 47;
FW-H3 the amino acid sequence of SEQ ID NO: 48;
FW-H4 the amino acid sequence of SEQ ID NO: 49.

In one embodiment all the amino acid substitutions are conservative amino acid substitutions. In a further embodiment each of said conservative amino acid substitutions is the substitution of an amino acid with another amino acid selected from its same group, wherein the groups of amino acids are
a) the nonpolar, hydrophobic amino acids consisting of Gly, Ala, Val, Leu, Ile, Phe, Try, Trp, and Met;
b) the polar, neutral amino acids consisting of Ser, Thr, Asn, and Gln;
c) the positively charged, basic amino acids consisting of Arg, Lys, and His, and
d) the negatively charged, amino acids consisting of Asp and Glu
wherein if Cys is to be conservatively substituted, it is substituted with Ser or Ala, and
wherein if Pro is to be conservatively substituted it is substituted with Ala.

In a further specific embodiment each of said conservative amino acid substitutions is a highly conservative amino acid substitution selected from
a) substitution of Ala with Val, Leu, Ile or Gly;
b) substitution of Arg with Lys;
c) substitution of Asn with Gln;
d) substitution of Asp with Glu;
e) substitution of Cys with Ser;
f) substitution of Gln with Asn;
g) substitution of Glu with Asp;
h) substitution of Gly with Ala;
i) substitution of His with Arg;
j) substitution of Ile with Leu, Val or Ala;
k) substitution of Leu with Ile, Val or Ala;
l) substitution of Lys with Arg;
m) substitution of Met with Leu, Ile or Val;
n) substitution of Phe with Try or Trp;
o) substitution of Pro with Ala;
p) substitution of Ser with Thr;
q) substitution of Thr with Ser;
r) substitution of Trp with Phe or Tyr;
s) substitution of Tyr with Phe or Trp; and
t) substitution of Val with Leu, Ile or Ala.

In a further embodiment of the eighteenth aspect or embodiments relating to the same the antibody or antigen binding fragment thereof binds to Pepsinogen C consisting of SEQ ID NO: 62 with a K_{D} of 0.6 nM or less, preferably wherein the K_{D} is measured at 37 °C.

In a further embodiment of the eighteenth aspect or embodiments relating to the same the antibody or antigen binding fragment thereof binds to Pepsinogen C consisting of SEQ ID NO: 62 with t/2-diss at 37 °C of 75 minutes or longer.

In a further embodiment of the eighteenth aspect or embodiments relating to the same the antibody or antigen binding fragment thereof has the association rate kₐ for Pepsinogen C consisting of SEQ ID NO: 62 of at least 2.6×10⁵ M⁻¹s⁻¹.

In a nineteenth aspect of the present invention the invention relates to an antibody or antigen binding fragment thereof comprising
(i) a heavy chain variable domain (VH) that is at least 80% homologous to the VH as shown in SEQ ID NO: 9; and/or
(ii) a light chain variable domain (VL) that is at least 80% homologous to the VL as shown in SEQ ID NO: 10.

In one embodiment the antibody or antigen binding fragment thereof according to the invention comprises
(i) a VH that is at least 85% homologous to the VH as shown in SEQ ID NO: 9; and/or
(ii) a VL that is at least 85% homologous to the VL as shown in SEQ ID NO: 10.

In one embodiment the antibody or antigen binding fragment thereof according to the invention comprises
(i) a VH that is at least 90% homologous to the VH as shown in SEQ ID NO: 9; and/or
(ii) a VL that is at least 90% homologous to the VL as shown in SEQ ID NO: 10.

In one embodiment the antibody or antigen binding fragment thereof according to the invention comprises
(i) a VH that is at least 95% homologous to the VH as shown in SEQ ID NO: 9; and/or
(ii) a VL that is at least 95% homologous to the VL as shown in SEQ ID NO: 10.

In one embodiment the antibody or antigen binding fragment thereof according to the invention comprises
(i) a VH comprising a sequence as shown in SEQ ID NO: 9; and/or
(ii) a VL comprising a sequence as shown in SEQ ID NO: 10.

In one embodiment the antibody or antigen binding fragment thereof according to the invention comprises
(i) a VH consisting of the sequence as shown in SEQ ID NO: 9; and/or
(ii) a VL consisting of the sequence as shown in SEQ ID NO: 10.

In an twentieth aspect the invention relates to an antibody or antigen binding fragment thereof that specifically binds to Pepsinogen C, comprising
(i) a VH comprising a CDR-H1 as shown in the amino acid sequence of SEQ ID NO: 11 or a variant thereof modified by one amino acid substitution; a CDR-H2 as shown in the amino acid sequence of SEQ ID NO: 12, or a variant thereof modified by one amino acid substitution; and a CDR-H3 as shown in the amino acid sequence of SEQ ID NO: 13, or a variant thereof modified by one amino acid substitution; and
(ii) a VL comprising a CDR-L1 as shown in the amino acid sequence of SEQ ID NO: 14 or a variant thereof modified by one amino acid substitution; a CDR-L2 as shown in the amino acid sequence of SEQ ID NO: 15, or a variant thereof modified by one amino acid substitution; and a CDR-L3 as shown in the amino acid sequence of SEQ ID NO: 16, or a variant thereof modified by one amino acid substitution.

In an embodiment the antibody or antigen binding fragment thereof according to the invention comprises
(i) a VH comprising a CDR-H1 as shown in the amino acid sequence of SEQ ID NO: 11; a CDR-H2 as shown in the amino acid sequence of SEQ ID NO: 12; and a CDR-H3 as shown in the amino acid sequence of SEQ ID NO: 13; and
(ii) a VL comprising a CDR-L1 as shown in the amino acid sequence of SEQ ID NO: 14; a CDR-L2 as shown in the amino acid sequence of SEQ ID NO: 15; and a CDR-L3 as shown in the amino acid sequence of SEQ ID NO: 16.

In a further embodiment the antibody or antigen binding fragment thereof according to the twentieth aspect of the invention comprises a VL consisting of framework regions (FW) and CDRs as represented in formula I:

(FW-L1)-(CDR-L1)-(FW-L2)-(CDR-L2)-(FW-L3)-(CDR-L3)-(FW-L4) (formula I)
and a VH consisting of FWs and CDRs as represented in formula II:

(FW-H1)-(CDR-H1)-(FW-H2)-(CDR-H2)-(FW-H3)-(CDR-H3)-(FW-H4) (formula II),
wherein the FWs comprise the following amino acid sequences or a variant thereof that is at least 85% homologous thereto:
FW-L1 the amino acid sequence of SEQ ID NO: 26;
FW-L2 the amino acid sequence of SEQ ID NO: 27;
FW-L3 the amino acid sequence of SEQ ID NO: 28;
FW-L4 the amino acid sequence of SEQ ID NO: 29;
FW-H1 the amino acid sequence of SEQ ID NO: 30;
FW-H2 the amino acid sequence of SEQ ID NO: 31;
FW-H3 the amino acid sequence of SEQ ID NO: 32;
FW-H4 the amino acid sequence of SEQ ID NO: 33.

In a further embodiment the antibody or antigen binding fragment thereof according to the twentieth aspect of the invention comprises a VL consisting of framework regions (FW) and CDRs as represented in formula I:

(FW-L1)-(CDR-L1)-(FW-L2)-(CDR-L2)-(FW-L3)-(CDR-L3)-(FW-L4) (formula I)
and a VH consisting of FWs and CDRs as represented in formula II:

(FW-H1)-(CDR-H1)-(FW-H2)-(CDR-H2)-(FW-H3)-(CDR-H3)-(FW-H4) (formula II),
wherein the FWs comprise the following amino acid sequences or a variant thereof modified by at most three amino acid substitutions:
FW-L1 the amino acid sequence of SEQ ID NO: 26;
FW-L2 the amino acid sequence of SEQ ID NO: 27;
FW-L3 the amino acid sequence of SEQ ID NO: 28;
FW-L4 the amino acid sequence of SEQ ID NO: 29;
FW-H1 the amino acid sequence of SEQ ID NO: 30;
FW-H2 the amino acid sequence of SEQ ID NO: 31;
FW-H3 the amino acid sequence of SEQ ID NO: 32;
FW-H4 the amino acid sequence of SEQ ID NO: 33.

In one embodiment all the amino acid substitutions are conservative amino acid substitutions. In a further embodiment each of said conservative amino acid substitutions is the substitution of an amino acid with another amino acid selected from its same group, wherein the groups of amino acids are
a) the nonpolar, hydrophobic amino acids consisting of Gly, Ala, Val, Leu, Ile, Phe, Try, Trp, and Met;
b) the polar, neutral amino acids consisting of Ser, Thr, Asn, and Gln;
c) the positively charged, basic amino acids consisting of Arg, Lys, and His, and
d) the negatively charged, amino acids consisting of Asp and Glu
wherein if Cys is to be conservatively substituted, it is substituted with Ser or Ala, and
wherein if Pro is to be conservatively substituted it is substituted with Ala.

In a further specific embodiment each of said conservative amino acid substitutions is a highly conservative amino acid substitution selected from
a) substitution of Ala with Val, Leu, Ile or Gly;
b) substitution of Arg with Lys;
c) substitution of Asn with Gln;
d) substitution of Asp with Glu;
e) substitution of Cys with Ser;
f) substitution of Gln with Asn;
g) substitution of Glu with Asp;
h) substitution of Gly with Ala;
i) substitution of His with Arg;
j) substitution of Ile with Leu, Val or Ala;
k) substitution of Leu with Ile, Val or Ala;
l) substitution of Lys with Arg;
m) substitution of Met with Leu, Ile or Val;
n) substitution of Phe with Try or Trp;
o) substitution of Pro with Ala;
p) substitution of Ser with Thr;
q) substitution of Thr with Ser;
r) substitution of Trp with Phe or Tyr;
s) substitution of Tyr with Phe or Trp; and
t) substitution of Val with Leu, Ile or Ala.

In a further embodiment of the nineteenth or twentieth aspect of the invention or embodiments relating to the same the antibody or antigen binding fragment thereof binds to Pepsinogen C consisting of SEQ ID NO: 62 with a KD of 0.3 nM or less, preferably wherein the K_{D} is measured at 37 °C.

In a further embodiment of the nineteenth or twentieth aspect of the invention or embodiments relating to the same the antibody or antigen binding fragment thereof binds to Pepsinogen C consisting of SEQ ID NO: 62 with t/2-diss at 37 °C of 30 minutes or longer.

In a further embodiment of the nineteenth or twentieth aspect of the invention or embodiments relating to the same the antibody or antigen binding fragment thereof has a association rate kₐ for Pepsinogen C consisting of SEQ ID NO: 62 of at least 1.4×10⁶ M⁻¹s⁻¹.

In a preferred embodiment the antibody or antigen binding fragment thereof according to the invention is a monoclonal antibody.

In a further embodiment of the invention the antibody or antigen binding fragment thereof specifically binds to Pepsinogen C, wherein the Pepsinogen C comprises SEQ ID NO: 17. In one embodiment the Pepsinogen C consists of SEQ ID NO: 17.

In a further embodiment of the invention, the antibody or antigen binding fragment thereof does not specifically bind to Pepsinogen A. That means that the antibody or antigen binding fragment thereof binds to Pepsinogen A with a KD greater than 100 nM. In a preferred embodiment, the Pepsinogen A comprises SEQ ID NO: 64 or a variant thereof comprising a E58K, K222Q, T265A or V353L amino acid substitution.

As demonstrated in the appended examples (e.g. Examples 5 and 9), the antibody of the invention is also characterized by having a high affinity to Pepsinogen C consisting of SEQ ID NO: 62. Accordingly, the antibody of the invention may be characterized by having an equilibrium dissociation constant K_{D} of 2 nM or less, 1.5 nM or less, 1 nM or less, or 0.6 nM or less. In one preferred embodiment the K_{D} is measured at 37 °C. Decreasing KD is preferred. The lower the KD, the more preferred.

In a preferred embodiment, the K_{D} of the antibody or antigen binding fragment thereof according to the invention for the binding to Pepsinogen C is determined by surface plasmon resonance spectroscopy. The experimental settings are described herein in Example 9.

Methods for determining an equilibrium dissociation constant KD are known in the art.

In a preferred embodiment, the K_{D} may be determined by surface plasmon resonance spectroscopy (e.g. BIAcore®).

The preferred temperature for determining the KD is 37 °C.

In embodiments, the K_{D} may be determined using kinetic measurements determining the association and dissociation rate. Accordingly, the methods may in embodiments be the same surface plasmon resonance methods as described for determination of the kₐ, below. Said method may comprise correction for mass transport limitation.

In preferred embodiments, the K_{D} may be determined using surface plasmon resonance spectroscopy (e.g. BIAcore®).

In a further embodiment the antibody or antigen binding fragment thereof according to the invention has an antibody/antigen complex half-life t_{/2-diss} at 37 °C of 20 minutes or longer, preferably 30 minutes or longer.

Methods for determining the antibody/antigen complex half-life of an antibody for the binding to Pepsinogen C are known in the art. Exemplary examples are described in the Examples (in particular Example 9).

The antibody/antigen complex half-life was calculated in minutes according to the formula t_{/2 diss}=ln(2)/(k_{d}*60).

As demonstrated in the appended Examples, the antibody of the invention is characterized by a particularly high association rate constant (kₐ) for the binding to Pepsinogen C. This fast association rate makes the antibody of the invention particularly suitable for high throughput immunoassay and a competitive assay format.

Methods for determining the association rate constant (kₐ) of an antibody for the binding to Pepsinogen C are known in the art. Exemplary examples are described in the Examples (in particular Example 9).

Accordingly, in embodiments the antibody or antigen binding fragment thereof according to the invention is further characterized in that the association rate (kₐ) for the binding to Pepsinogen C consisting of SEQ ID NO: 62 is at least 10⁵ M⁻¹s⁻¹, preferable at least 2.5×10⁵ M⁻¹s⁻¹. In one preferred embodiment, the association rate for the binding to Pepsinogen C consisting of SEQ ID NO: 62 is at least 2.5×10⁵ M⁻¹s⁻¹. In one embodiment kₐ is measured at 37 °C.

Methods for determining the association rate constant (kₐ) of an antibody for the binding to Pepsinogen C are known in the art. Exemplary examples are described in the Examples (in particular Example 9).

In a preferred embodiment, the association rate constant (kₐ) of the antibody or antigen binding fragment thereof according to the invention for the binding to Pepsinogen C is determined by surface plasmon resonance spectroscopy.

Said surface plasmon resonance spectroscopy comprises attaching or capturing the antibody, or antigen binding fragment thereof, on a CM5 sensor chip and injecting Pepsinogen C comprising SEQ ID NO: 17 as analyte, wherein said determination is conducted at a temperature of 37 °C using HBS-ET buffer (10 mM HEPES, 150 mM NaCl, 3 mM EDTA, 0.05% (w/v) Tween 20®).

In one embodiment the surface plasmon resonance spectroscopy is performed with a Biacore 8 k or B4000 instrument.

The association constant kₐ [1/Ms], the dissociation rate constant k_{d} [s⁻¹] and the dissociation equilibrium constant K_{D} [M] may be calculated according to a Langmuir fitting model, e.g. by using the evaluation software corresponding to the instrument.

The antibodies and antigen binding fragments of the invention may be prepared by a variety of techniques routinely used in the art. For example, antibodies can be prepared immunizing a non-human animal (e.g. mice) with Pepsinogen C isolating and subsequently isolating antigen-reactive, antibody producing B-cells. Preferred exemplary methods for the production of antibodies according to the invention using immunization of non-human animals are provided in the appended Examples (see in particular Example 3). Selected clones for producing antibodies can be processed according to routine methods for subsequent recombinant processing.

Another suitable method for producing or isolating antibodies and antibody antigen binding fragments of the invention include, but are not limited to, methods that select a recombinant antibody from a peptide or protein library (e.g., but not limited to, a bacteriophage, ribosome, oligonucleotide, RNA, cDNA, or yeast display library) using binding activities of interest. For example, antibodies or antigen binding fragments can be selected from such libraries by positively selecting for specific binding to Pepsinogen C. Display libraries are well known in the art and are, for example, available from various commercial vendors including but not limited to Cambridge Antibody Technologies (Cambridgeshire, UK), MorphoSys (Martinsried/Planegg, Del.), Biovation (Aberdeen, Scotland, UK) and Bioinvent (Lund, Sweden). Again, selected clones can be processed according to routine methods for subsequent recombinant processing.

Antibodies of the invention may be recombinant expressed. Accordingly, in certain embodiments, the antibody of the invention may be a recombinant antibody. Methods for producing a recombinant antibody are known in the art.

In a twenty-first aspect the invention relates to a polynucleotide or a set of polynucleotides encoding
(i) the heavy chain variable domain of the antibody or antigen binding fragment thereof according to the invention, and
(ii) the light chain variable domain of the antibody or antigen binding fragment according to the invention.

The present invention also provides a nucleic acid molecule encoding the antibody of the invention or any antigen-binding fragments thereof, as defined herein above. In particular provided is a polynucleotide encoding a heavy chain and/or light chain variable domain of the monoclonal antibody specifically binding to Pepsinogen C as defined herein above. In some embodiments, the polynucleotide may comprise further sequences to ensure that not only the heavy and/or light chain variable domain are expressed, but also the remaining heavy and/or light chain constant regions such that a full-length IgG antibody is expressed comprising the heavy and light chain variable domains of the invention. Accordingly, for each of the aspects and embodiments relating to antibodies or antigen binding fragments specifically binding Pepsinogen C as described herein a corresponding polynucleotide encoding the respective antibody or antigen binding fragment is provided herein.

In a twenty-second aspect, provided herein is a vector that comprises a polynucleotide of the invention. In particular, provided are vectors comprising a nucleic acid molecule encoding an antibody or antibody antigen binding fragment of the invention. As used herein, the term “vector” relates to a circular or linear nucleic acid molecule that can autonomously replicate in a host cell into which it has been introduced. Non-limiting examples of vectors suitable for use in the present invention include cosmids, plasmids (e.g., naked or contained in liposomes), viruses (e.g., lentiviruses, retroviruses, adenoviruses, and adeno-associated viruses) and bacteriophages. However, the art provides many suitable vectors, the choice of which depends on the desired function. The development and use of suitable vectors is well documented in the art; see, for example, the techniques described in Sambrook and Russel “Molecular Cloning, A Laboratory Manual”, Cold Spring Harbor Laboratory, N.Y. (2001) and Ausubel, “Current Protocols in Molecular Biology”, Green Publishing Associates and Wiley Interscience, N.Y. (1989), (1994). Vectors of use in connection with the present invention comprise a nucleic acid sequence encoding the full length Pepsinogen C antibody antigen binding fragment as disclosed herein. As such, for each of the aspects and embodiments relating to antibodies or antigen binding fragments specifically binding Pepsinogen C as described herein a vector comprising the corresponding polynucleotide encoding the respective antibody or antigen binding fragment is provided herein.

With regard to the term “vector comprising” as used herein, it is understood in the art that further nucleic acid sequences are present in the vectors that are necessary and/or sufficient for desired vector activity in the host cell, e.g. drive replication of the vector (and, thus the encoding nucleic acid sequences) and/or to direct the host cell express the antibody or antigen binding fragment of the invention. Such further nucleic acid sequences include but are not limited to sequences controlling vector replication and/or expression of a desired sequence in the particular cell system. For example, the vectors may comprise the nucleic acid molecule encoding an antibody or antibody antigen binding fragment of the invention operably linked and/or under the control of regulatory sequences. The term “regulatory sequence” refers to DNA sequences that are necessary to effect the expression of coding sequences to which they are operably linked. The term “control sequence” is intended to include, at a minimum, all components the presence of which may also be necessary for expression, and may further include additional advantageous components, e.g., to allow replication. As is understood in the art, the nature of such regulatory and control sequences differs depending upon the host organism. For example, in prokaryotes, control sequences generally include promoters, ribosomal binding sites, and terminators. In eukaryotes control sequences generally include promoters, terminators and, in some instances, enhancers, transactivators and/or transcription factors.

The vectors of use in the present invention are preferably expression vectors. An expression vector is capable of directing the replication and the expression of the nucleic acid molecule of the invention in a host cell and, accordingly, provides for the expression of, e.g., the heavy chain and/or light chain variable domains of the antibodies specifically binding to Pepsinogen C as disclosed herein. In some embodiments, the vector may comprise further sequences to ensure that not only the heavy and light chain variable domains are expressed, but also the remaining heavy and light chain constant regions such that a full-length IgG antibody is expressed comprising the heavy and light chain variable domains of the invention. Suitable expression vectors have been widely described in the literature and the determination of the appropriate expression vector for a particular cell system can be readily made by the skilled person using routine methods. Preferably, the vectors disclosed herein comprise a recombinant polynucleotide (i.e., a nucleic acid sequence encoding the monoclonal antibody according to the invention) as well as expression operably linked control sequences. The vectors as provided herein preferably further comprise a promoter. The herein described vectors may also comprise a selection marker gene and a replication-origin ensuring replication in the host Moreover, the herein provided vectors may also comprise a termination signal for transcription. Expression vectors as known in the art may drive transient or constitutive expression in a host cell.

The nucleic acid molecules and/or vectors of the invention can be designed for transfection into prokaryotic or eukaryotic host cells by any means known in the art or described herein. Non-limiting examples of suitable methods include chemical based methods (polyethylenimine, calcium phosphate, liposomes, DEAE-dextrane, nucleofection), nonchemical methods (electroporation, sonoporation, optical transfection, gene electrotransfer, hydrodynamic delivery or naturally occurring transformation upon contacting cells with the nucleic acid molecule of the invention), particle-based methods (gene gun, magnetofection, impalefection) phage vector-based methods and viral methods. For example, expression vectors derived from viruses such as retroviruses, vaccinia virus, adeno-associated virus, herpes viruses, Semliki Forest Virus or bovine papilloma virus, may be used for transfection of the nucleic acid molecules into targeted cell population. Additionally, baculoviral systems can also be used as vector in eukaryotic expression system for the nucleic acid molecules of the invention.

The term “prokaryote” is meant to include all bacteria which can be transformed, transduced or transfected with DNA or DNA or RNA molecules for the expression of a protein of the invention. Prokaryotic hosts may include gram negative as well as gram positive bacteria such as, for example, E. coli, S. typhimurium, Serratia marcescens, Corynebacterium (glutamicum), Pseudomonas (fluorescens), Lactobacillus, Streptomyces, Salmonella and Bacillus subtilis. The term “eukaryotic” is meant to include yeast, higher plant, insect and mammalian cells. Non-limiting examples of mammalian host cells typically used in the art include, Hela, HEK293, H9, Per.C6 and Jurkat cells, mouse NIH3T3, NS/0, SP2/0 and C127 cells, COS cells, e.g. COS 1 or COS 7, CV1, quail QC1-3 cells, mouse L cells, mouse sarcoma cells, Bowes melanoma cells and Chinese hamster ovary (CHO) cells.

Accordingly, in a twenty-third aspect the present invention relates to a host cell comprising a polynucleotide according to the invention, or a vector according to the invention. The host cell may be a prokaryotic cell or a eukaryotic cell. In a preferred embodiment, the host cell is a eukaryotic cell. In a particular embodiment, the cell is a HEK cell. In another particular embodiment the host cell is a CHO cell.

When recombinant expression vectors encoding the heavy and/or light chain of the antibody of the invention as disclosed herein are introduced into host cells, the antibodies or antibody antigen binding fragments are produced by culturing the host cells for a period of time sufficient to allow for expression of the antibody or antigen binding fragment in the host cell or, preferably, to allow for secretion of the antibody or antigen binding fragment into the culture medium in which the host cells are grown. Antibodies and/or antigen binding fragments can be recovered from the culture medium using standard protein purification methods. Methods for purification of antibodies are well known in the art. Exemplary purification methods are described in the appended Examples.

Accordingly, the invention also provides in a twenty-fourth aspect a method for the production of an antibody specifically binding to Pepsinogen C as disclosed herein. The method comprises culturing a host cell of the invention under suitable conditions and isolating the antibody produced. By purification steps, e.g. as described in the appended Examples an isolated antibody of the invention can be derived.

The invention further provides in a twenty-fifth aspect an antibody or an antigen binding fragment obtainable by any of the methods disclosed herein.

The transformed host cells can be grown in bioreactors and cultured according to techniques known in the art to achieve optimal cell growth. The antibody and/or antibody antigen binding fragment of the invention can then be isolated from the cell fraction or growth medium by any conventional means such, but not limited to, affinity chromatography (for example using a fusion-tag such as the Strep-tag II or the His₆ tag), gel filtration (size exclusion chromatography), anion exchange chromatography, cation exchange chromatography, hydrophobic interaction chromatography, high pressure liquid chromatography (HPLC), reversed phase HPLC or immunoprecipitation.

It will be appreciated that variations on the above procedures are within the scope of the present invention. For example, recombinant DNA technology may be used to remove or modify the DNA sequences encoding the antibodies and/or antibody antigen binding fragments disclosed herein, e.g. encoding the heavy and/or light chain variable domains as defined herein above. For example, recombinant DNA technology may be used to remove parts of the encoding sequence(s) that are not necessary for maintaining specific and selective binding to the antigen(s) of interest. The molecules expressed from such truncated DNA molecules are also encompassed by the antibodies of the invention.

Antibody derivatives can be produced, for example, by adding exogenous sequences to modify immunogenicity or reduce, enhance or modify binding, affinity, on-rate, off-rate, avidity, specificity, half-life, or any other suitable characteristic.

Also provided are humanized versions of the antibodies disclosed herein, i.e. comprising the CDRs of the heavy and or light chains as disclosed herein above. As well known in the art, “humanization” (to produce a humanized version of a parent antibody) refers to recombinantly engineering an antibody using CDRs derived from a non-human donor immunoglobulin in the context of human derived framework and constant domains. During the engineering, framework and/or CDR residues may be altered to preserve binding affinity and activity, e.g. specificity for Pepsinogen C. Methods to humanize antibodies are well known in the art, e.g. as disclosed in Queen et al., Proc. Natl. Acad Sci USA 86(1989), 10029-10032; Hodgson et al., Bio/Technology 9(1991) 421.

In a twenty-sixth aspect the invention provides a composition comprising an antibody of the invention, a polynucleotide of the invention, a vector of the invention, or a host cell of the invention. In a preferred embodiment, the composition is a diagnostic composition, i.e. a composition for use in diagnostic applications. In preferred embodiments, the composition is for use in an in vitro diagnostic test for detecting Pepsinogen C. In a preferred embodiment the diagnostic composition may be a reagent for an immunoassay for detecting Pepsinogen C. The diagnostic composition is preferably configured such that it allows for detection of Pepsinogen C in a sample obtained from a subject. The sample is preferably a blood sample (e.g. whole blood, serum or plasma).

In a twenty-seventh aspect the invention provides a composition comprising
a) the antibody or antigen binding fragment thereof according to the sixteenth aspect of the invention or according to any embodiment relating to the sixteenth aspect and
   the antibody or antigen binding fragment thereof according the nineteenth aspect of the invention or according to any embodiment relating to the nineteenth aspect.

Also provided is a composition comprising a polynucleotide, a vector or a host cell relating to the antibody of the sixteenth aspect of the invention or embodiments thereof and a polynucleotide, a vector or a host cell relating to the antibody of the nineteenth aspect of the invention or embodiments thereof.

In one embodiment the composition according to the invention is a diagnostic composition.

In particular the invention provides a composition comprising
the antibody or antigen binding fragment thereof according to the sixteenth aspect of the invention or to embodiments of the sixteenth aspect; and
the antibody or antigen binding fragment thereof according to the nineteenth aspect of the invention or to embodiments of the nineteenth aspect;
wherein the composition is a diagnostic composition.

As already discussed above it is demonstrated in Example 6 that the antibodies of the invention were each labeled with one label. Two sets of antibodies were prepared. One set of antibodies were labeled with a Ruthenium label and the second set of antibodies were labeled with a Biotin label. In Example 7 different combinations of antibodies were tested. A Pepsinogen C containing sample was incubated with ruthenylated monoclonal Pepsinogen C-specific IgG antibody and biotinylated monoclonal Pepsinogen C-specific IgG antibody and thereby form a sandwich complex. As demonstrated in Table 1 the inventors surprisingly identified antibodies which in combination show a surprisingly high signal (e.g. antibodies like mAb22 or antibodies binding the same epitope (mAb27, 75 or 123) combined with mAb11) compared to other combinations (e.g. antibodies like mAb22 or antibodies binding the same epitope (mAb27, 75 or 123) combined with mAb46). Which combination of antibodies gives a particularly good signal was very surprising. Without being bound by theory it seems that the epitopes bound by the antibodies influence which combinations of antibodies are advantageous (see Table 2).

In a twenty-eight aspect the invention relates to the use of the antibody or antigen binding fragment thereof according to the invention or the composition according to the invention for an in vitro immunoassay, in particular an in vitro immunoassay for detecting Pepsinogen C in a sample. Accordingly, also provided is an antibody of the invention for use in an immunoassay (e.g. a heterogeneous immunoassay) for detecting Pepsinogen C.

The sample may be a body fluid, such as, but not restricted to, a blood sample, cerebrospinal fluid, seminal fluid, saliva or urine. In embodiments, the sample is a blood sample, such as whole blood, serum or plasma. In embodiments, the sample is serum or plasma.

The method for detecting may comprise (a) contacting a sample comprising Pepsinogen C with the antibody, or antigen binding fragment thereof, according to the invention, thereby forming a complex comprising Pepsinogen C and said antibody, or antigen binding fragment thereof, and (b) detecting the complex formed in step (a), thereby detecting Pepsinogen C in said sample.

In one embodiment the Pepsinogen C in the sample comprises a sequence that is at least 95% homologous to the sequence as shown in SEQ ID NO: 17.

In one specific embodiment the method is a serum immunoassay, wherein the sample is a body fluid, preferably whole blood, serum or plasma.

In one specific embodiment the antibody, or antigen binding fragment thereof, has a detection label attached thereto.

The detection label linked to the antibody of the invention may be any detection label known in the art. For example, the detection label may selected from an enzyme, a label emitting light, preferable fluorescence, luminescence, chemiluminescence, electrochemiluminescence, or radioactivity.

In a twenty-ninth aspect the invention provides an in vitro method for detecting Pepsinogen C in a sample, comprising the following steps:
1) contacting a sample comprising Pepsinogen C with a first antibody, or antigen binding fragment thereof, and a second antibody, or antigen binding fragment thereof, wherein
(a) the first antibody is
   (i) the antibody or antigen binding fragment thereof according to the sixteenth aspect of the invention or to embodiments of the sixteenth aspect; or
   (ii) the antibody or antigen binding fragment thereof according to the nineteenth aspect of the invention or to embodiments of the nineteenth aspect; and
(b) the second antibody specifically binds to Pepsinogen C and does not compete with the first antibody for specifically binding to Pepsinogen C; and
2) incubating the resulting mixture of 1) to form a complex comprising Pepsinogen C and said first antibody or antigen binding fragment thereof and said second antibody or antigen binding fragment thereof; and
3) detecting Pepsinogen C in the sample by detecting the formation of the complex as defined in 2).

As already discussed above it is demonstrated in Example 6 that the antibodies of the invention were each labeled with one label. Two sets of antibodies were prepared. One set of antibodies were labeled with a Ruthenium label and the second set of antibodies were labeled with a Biotin label. In Example 7 different combinations of antibodies were tested. A Pepsinogen C containing sample was incubated with ruthenylated monoclonal Pepsinogen C-specific IgG antibody and biotinylated monoclonal Pepsinogen C-specific IgG antibody and thereby form a sandwich complex. As demonstrated in Table 1 the inventors surprisingly identified antibodies which in combination show a surprisingly high signal (e.g. antibodies like mAb22 or antibodies binding the same epitope (mAb27, 75 or 123) combined with mAb11) compared to other combinations (e.g. antibodies like mAb22 or antibodies binding the same epitope (mAb27, 75 or 123) combined with mAb46). Which combination of antibodies gives a particularly good signal was very surprising. Without being bound by theory it seems that the epitopes bound by the antibodies influence which combinations of antibodies are advantageous (see Table 2).These advantageous combinations of antibodies can be used in the in vitro method for detecting Pepsinogen C in a sample.

In one embodiment of the twenty-ninth aspect of the invention (c) one of the first and the second antibody comprises a detection label.

In a specific embodiment the first antibody or antigen binding fragment thereof or the second antibody or antigen binding fragment thereof not comprising the detection label, comprises a capture label.

“Capture labels” as used herein relates to labels that can immobilize a capture agent (e.g. an antibody according to the invention having a capture label attached thereto) on a surface (e.g., on a magnetic particle such as a microbead). Non-limiting examples are members of binding pairs. A non-limiting example for a capture label is biotin or derivatives thereof, which can interact with streptavidin or derivatives thereof. Different capture labels are well known in the art.

In one specific embodiment the method further comprises adding to the mixture of (1) particles capable of binding to the capture label and 2.1) separating the particles from the mixture before detecting a signal provided by the detection label.

In one embodiment
when the first antibody, or antigen binding fragment thereof, is selected from (i) than the second antibody is selected from (ii); or
when the first antibody, or antigen binding fragment thereof, is selected from (ii) than the second antibody is selected from (i).

In one embodiment the Pepsinogen C in the sample comprises a sequence that is at least 95% homologous to the sequence as shown in SEQ ID NO: 17.

In a specific embodiment the method is a serum immunoassay, wherein the sample is a body fluid, preferably whole blood, serum or plasma.

The detection label linked to the antibody of the invention may be any detection label known in the art. For example, the detection label may selected from an enzyme, a label emitting light, preferable fluorescence, luminescence, chemiluminescence, electrochemiluminescence, or radioactivity

In a further embodiment the Limit of Quantification (LoQ) of the method of the invention is 0.353 ng/mL at 20% CV.

The LoQ is defined as the lowest amount of analyte in a sample that can be accurately quantitated with an intermediate precision CV of ≤20%.

In a further embodiment the detection of Pepsinogen C in the sample is achieved in 18 minutes or less.

In a thirtieth aspect provided herein is a kit comprising the antibody of the invention or a composition comprising the same. In embodiments, the kit is a kit for detecting and/or quantifying Pepsinogen C in vitro. In embodiments, the kit is an immunoassay kit. In embodiments, the kit is a kit for a heterogenous immunoassay.

In a thirty-first aspect provided herein is a kit comprising the composition according to the tenth aspect of the invention, or a first antibody or antigen binding fragment thereof and a second antibody or antigen binding fragment thereof wherein one of the first or second antibody or antigen binding fragment thereof is the antibody of the sixteenth aspect of the invention or an embodiment of the sixteenth aspect and the other of the first or second antibody or antigen binding fragment thereof is the antibody of the nineteenth aspect of the invention or an embodiment of the nineteenth aspect.

As already discussed above it is demonstrated in Example 6 that the antibodies of the invention were each labeled with one label. Two sets of antibodies were prepared. One set of antibodies were labeled with a Ruthenium label and the second set of antibodies were labeled with a Biotin label. In Example 7 different combinations of antibodies were tested. A Pepsinogen C containing sample was incubated with ruthenylated monoclonal Pepsinogen C-specific IgG antibody and biotinylated monoclonal Pepsinogen C-specific IgG antibody and thereby form a sandwich complex. As demonstrated in Table 1 the inventors surprisingly identified antibodies which in combination show a surprisingly high signal (e.g. antibodies like mAb22 or antibodies binding the same epitope (mAb27, 75 or 123) combined with mAb11) compared to other combinations (e.g. antibodies like mAb22 or antibodies binding the same epitope (mAb27, 75 or 123) combined with mAb46). Which combination of antibodies gives a particularly good signal was very surprising. Without being bound by theory it seems that the epitopes bound by the antibodies influence which combinations of antibodies are advantageous (see Table 2).

In one embodiment of the thirty-first aspect of the invention one of the first and the second antibody comprises a detection label.

In a specific embodiment the first antibody or antigen binding fragment thereof or the second antibody or antigen binding fragment thereof not comprising the detection label, comprises a capture label.

“Capture labels” as used herein relates to labels that can immobilize a capture agent (e.g. an antibody according to the invention having a capture label attached thereto) on a surface (e.g., on a magnetic particle such as a microbead). Non-limiting examples are members of binding pairs. A non-limiting example for a capture label is biotin or derivatives thereof, which can interact with streptavidin or derivatives thereof. Different capture labels are well known in the art.

In one specific embodiment the kit is immunoassay kit. In embodiments, the kit is a kit for a heterogeneous immunoassay.

In one embodiment the kit is for detecting and/or quantifying Pepsinogen C.

In a thirty-second aspect the invention provides a kit comprising a first antibody or antigen binding fragment thereof and a second antibody or antigen binding fragment thereof,
a) wherein the first antibody or antigen binding fragment thereof is
an antibody or antigen binding fragment thereof that specifically binds to Pepsinogen C and competes for binding to Pepsinogen C with the antibody consisting of
(i) a heavy chain variable domain (VH) as shown in SEQ ID NO: 1; and
(ii) a light chain variable domain (VL) as shown in SEQ ID NO: 2; and
(b) wherein the second antibody or antigen binding fragment thereof is
an antibody or antigen binding fragment thereof that specifically binds to Pepsinogen C and competes for binding to Pepsinogen C, consisting of
(iii) a heavy chain variable domain (VH) as shown in SEQ ID NO: 9; and
(iv) a light chain variable domain (VL) as shown in SEQ ID NO: 10; and
wherein the second antibody or antigen binding fragments thereof does not compete for binding to Pepsinogen C with the first antibody or antigen binding fragment thereof.

As already discussed above it is demonstrated in Example 6 that the antibodies of the invention were each labeled with one label. Two sets of antibodies were prepared. One set of antibodies were labeled with a Ruthenium label and the second set of antibodies were labeled with a Biotin label. In Example 7 different combinations of antibodies were tested. A Pepsinogen C containing sample was incubated with ruthenylated monoclonal Pepsinogen C-specific IgG antibody and biotinylated monoclonal Pepsinogen C-specific IgG antibody and thereby form a sandwich complex. As demonstrated in Table 1 the inventors surprisingly identified antibodies which in combination show a surprisingly high signal (e.g. antibodies like mAb22 or antibodies binding the same epitope (mAb27, 75 or 123) combined with mAb11) compared to other combinations (e.g. antibodies like mAb22 or antibodies binding the same epitope (mAb27, 75 or 123) combined with mAb46). Which combination of antibodies gives a particularly good signal was very surprising. Without being bound by theory, it seems that the epitopes bound by the antibodies influence which combinations of antibodies are advantageous (see Table 2).These advantageous combinations of antibodies can be used in the in vitro method for detecting Pepsinogen C in a sample.

In one embodiment the kit according to the invention comprises the first antibody or antigen binding fragment thereof comprising
(a) a VH comprising a CDR-H1 as shown in the amino acid sequence of SEQ ID NO: 3 or a variant thereof modified by one amino acid substitution; a CDR-H2 as shown in the amino acid sequence of SEQ ID NO: 4, or a variant thereof modified by one amino acid substitution; and a CDR-H3 as shown in the amino acid sequence of SEQ ID NO: 5, or a variant thereof modified by one amino acid substitution; and
a VL comprising a CDR-L1 as shown in the amino acid sequence of SEQ ID NO: 6 or a variant thereof modified by one amino acid substitution; a CDR-L2 as shown in the amino acid sequence of SEQ ID NO: 7, or a variant thereof modified by one amino acid substitution; and a CDR-L3 as shown in the amino acid sequence of SEQ ID NO: 52, or a variant thereof modified by one amino acid substitution, and
the second antibody or antigen binding fragment thereof comprising
(c) a VH comprising a CDR-H1 as shown in the amino acid sequence of SEQ ID NO: 11 or a variant thereof modified by one amino acid substitution; a CDR-H2 as shown in the amino acid sequence of SEQ ID NO: 12, or a variant thereof modified by one amino acid substitution; and a CDR-H3 as shown in the amino acid sequence of SEQ ID NO: 13, or a variant thereof modified by one amino acid substitution; and
VL comprising a CDR-L1 as shown in the amino acid sequence of SEQ ID NO: 14 or a variant thereof modified by one amino acid substitution; a CDR-L2 as shown in the amino acid sequence of SEQ ID NO: 15, or a variant thereof modified by one amino acid substitution; and a CDR-L3 as shown in the amino acid sequence of SEQ ID NO: 16, or a variant thereof modified by one amino acid substitution.

In another embodiment the kit according to the invention comprises the first antibody or antigen binding fragment comprising
(b) a VH comprising a CDR-H1 as shown in the amino acid sequence of SEQ ID NO: 36 or a variant thereof modified by one amino acid substitution; a CDR-H2 as shown in the amino acid sequence of SEQ ID NO: 37, or a variant thereof modified by one amino acid substitution; and a CDR-H3 as shown in the amino acid sequence of SEQ ID NO: 38, or a variant thereof modified by one amino acid substitution; and
a VL comprising a CDR-L1 as shown in the amino acid sequence of SEQ ID NO: 39 or a variant thereof modified by one amino acid substitution; a CDR-L2 as shown in the amino acid sequence of SEQ ID NO: 40, or a variant thereof modified by one amino acid substitution; and a CDR-L3 as shown in the amino acid sequence of SEQ ID NO: 41, or a variant thereof modified by one amino acid substitution; and
the second antibody or antigen binding fragment thereof comprising
(c) a VH comprising a CDR-H1 as shown in the amino acid sequence of SEQ ID NO: 11 or a variant thereof modified by one amino acid substitution; a CDR-H2 as shown in the amino acid sequence of SEQ ID NO: 12, or a variant thereof modified by one amino acid substitution; and a CDR-H3 as shown in the amino acid sequence of SEQ ID NO: 13, or a variant thereof modified by one amino acid substitution; and
VL comprising a CDR-L1 as shown in the amino acid sequence of SEQ ID NO: 14 or a variant thereof modified by one amino acid substitution; a CDR-L2 as shown in the amino acid sequence of SEQ ID NO: 15, or a variant thereof modified by one amino acid substitution; and a CDR-L3 as shown in the amino acid sequence of SEQ ID NO: 16, or a variant thereof modified by one amino acid substitution.

In one embodiment the kit according to the invention comprises the first antibody or antigen binding fragment thereof comprising
(a) a VH comprising a CDR-H1 as shown in the amino acid sequence of SEQ ID NO: 3 or a variant thereof modified by one amino acid substitution; a CDR-H2 as shown in the amino acid sequence of SEQ ID NO: 4, or a variant thereof modified by one amino acid substitution; and a CDR-H3 as shown in the amino acid sequence of SEQ ID NO: 5, or a variant thereof modified by one amino acid substitution; and
a VL comprising a CDR-L1 as shown in the amino acid sequence of SEQ ID NO: 6 or a variant thereof modified by one amino acid substitution; a CDR-L2 as shown in the amino acid sequence of SEQ ID NO: 7, or a variant thereof modified by one amino acid substitution; and a CDR-L3 as shown in the amino acid sequence of SEQ ID NO: 52, or a variant thereof modified by one amino acid substitution, or
the second antibody or antigen binding fragment thereof comprising
(c) a VH comprising a CDR-H1 as shown in the amino acid sequence of SEQ ID NO: 11 or a variant thereof modified by one amino acid substitution; a CDR-H2 as shown in the amino acid sequence of SEQ ID NO: 12, or a variant thereof modified by one amino acid substitution; and a CDR-H3 as shown in the amino acid sequence of SEQ ID NO: 13, or a variant thereof modified by one amino acid substitution; and
VL comprising a CDR-L1 as shown in the amino acid sequence of SEQ ID NO: 14 or a variant thereof modified by one amino acid substitution; a CDR-L2 as shown in the amino acid sequence of SEQ ID NO: 15, or a variant thereof modified by one amino acid substitution; and a CDR-L3 as shown in the amino acid sequence of SEQ ID NO: 16, or a variant thereof modified by one amino acid substitution.

In another embodiment the kit according to the invention comprises the first antibody or antigen binding fragment comprising
(b) a VH comprising a CDR-H1 as shown in the amino acid sequence of SEQ ID NO: 36 or a variant thereof modified by one amino acid substitution; a CDR-H2 as shown in the amino acid sequence of SEQ ID NO: 37, or a variant thereof modified by one amino acid substitution; and a CDR-H3 as shown in the amino acid sequence of SEQ ID NO: 38, or a variant thereof modified by one amino acid substitution; and
a VL comprising a CDR-L1 as shown in the amino acid sequence of SEQ ID NO: 39 or a variant thereof modified by one amino acid substitution; a CDR-L2 as shown in the amino acid sequence of SEQ ID NO: 40, or a variant thereof modified by one amino acid substitution; and a CDR-L3 as shown in the amino acid sequence of SEQ ID NO: 41, or a variant thereof modified by one amino acid substitution; or
the second antibody or antigen binding fragment thereof comprising
(c) a VH comprising a CDR-H1 as shown in the amino acid sequence of SEQ ID NO: 11 or a variant thereof modified by one amino acid substitution; a CDR-H2 as shown in the amino acid sequence of SEQ ID NO: 12, or a variant thereof modified by one amino acid substitution; and a CDR-H3 as shown in the amino acid sequence of SEQ ID NO: 13, or a variant thereof modified by one amino acid substitution; and
VL comprising a CDR-L1 as shown in the amino acid sequence of SEQ ID NO: 14 or a variant thereof modified by one amino acid substitution; a CDR-L2 as shown in the amino acid sequence of SEQ ID NO: 15, or a variant thereof modified by one amino acid substitution; and a CDR-L3 as shown in the amino acid sequence of SEQ ID NO: 16, or a variant thereof modified by one amino acid substitution.

In thirty-third aspect the invention relates to an antibody or antigen binding fragment thereof that specifically binds to Pepsinogen C and competes for binding to Pepsinogen C with the antibody consisting of (i) a heavy chain variable domain (VH) as shown in SEQ ID NO: 1; and (ii) a light chain variable domain (VL) as shown in SEQ ID NO: 2.

Pepsinogen C is secreted by the fundig glands, pyloric glands, and procimal duodenal mucosa (Samloff and Liebman 1973). Previous studies have shown that the serum PG I level and/or I/II ratio reflect the morphological (Samloff et al. 1982; Borch et al. 1989) and functional statuses (Samloff et al. 1975; Nakanome et al. 1983; Miki et al. 1987; Feldman et al. 1988) of the gastric mucosa. Many studies have reported a significant correlation between serum PG values and gastric acid secretion levels. Alteration of the gastric secretion level is related to the pathogenesis of various kinds of diseases originating from the upper gastrointestinal (GI) tract. Classically, individuals with hyperchlorhydria, especially those with Helicobacter pylon infection, are predisposed to the development of duodenal ulcers, whereas individuals with hypochlorhydria are at the risk of gastric cancer through the development of a premalignant condition, gastric atrophy (El-Omar et al. 1995, 1997) Thus, measurement of the gastric acid secretion level has some clinical implications in estimating the risk for various diseases in the upper GI tract. However, given that the procedure causes significant distress to the subjects and is time-consuming, direct measurement of the gastric acid secretion level has become less prevalent these days. Therefore, antibodies specifically binding to Pepsinogen C which is a useful indicator for the measurement of the gastric acid secretion level and for estimating the risk for various disease in the upper GI tract are needed.

The inventors of the present invention have identified high affinity antibodies specifically binding to Pepsinogen C and competing for binding to Pepsinogen C with the antibody consisting of (i) a heavy chain variable domain (VH) as shown in SEQ ID NO: 1; and (ii) a light chain variable domain (VL) as shown in SEQ ID NO: 2.

In one embodiment the antibody or antigen binding fragment thereof according to the invention comprises (a) a VH comprising a CDR-H1 as shown in the amino acid sequence of SEQ ID NO: 3 or a variant thereof modified by one amino acid substitution; a CDR-H2 as shown in the amino acid sequence of SEQ ID NO: 4, or a variant thereof modified by one amino acid substitution; and a CDR-H3 as shown in the amino acid sequence of SEQ ID NO: 5, or a variant thereof modified by one amino acid substitution; and a VL comprising a CDR-L1 as shown in the amino acid sequence of SEQ ID NO: 6 or a variant thereof modified by one amino acid substitution; a CDR-L2 as shown in the amino acid sequence of SEQ ID NO: 7, or a variant thereof modified by one amino acid substitution; and a CDR-L3 as shown in the amino acid sequence of SEQ ID NO: 52, or a variant thereof modified by one amino acid substitution.

The present inventors surprisingly identified a family of antibodies which compete for specifically binding Pepsinogen C. As demonstrated in the appended Examples (e.g. Example 8, Table 1) the antibodies of this family (e.g. mAb22, 27, 75 and 123) all compete for binding to Pepsinogen C. As can be seen in FIG. 5 the CDRs, which are i.a. responsible for the binding to the respective antigen, show very similar amino acid sequences. The CDR-L3 of three antibodies only show one amino acid variation, as shown in the consensus sequence SEQ ID NO: 52 (QQDXKYPWT, wherein X is the amino acid Val or Ile).

In a further embodiment the antibody or antigen binding fragment thereof according to the invention comprises (b) a VH comprising a CDR-H1 as shown in the amino acid sequence of SEQ ID NO: 36 or a variant thereof modified by one amino acid substitution; a CDR-H2 as shown in the amino acid sequence of SEQ ID NO: 37, or a variant thereof modified by one amino acid substitution; and a CDR-H3 as shown in the amino acid sequence of SEQ ID NO: 38, or a variant thereof modified by one amino acid substitution; and a VL comprising a CDR-L1 as shown in the amino acid sequence of SEQ ID NO: 39 or a variant thereof modified by one amino acid substitution; a CDR-L2 as shown in the amino acid sequence of SEQ ID NO: 40, or a variant thereof modified by one amino acid substitution; and a CDR-L3 as shown in the amino acid sequence of SEQ ID NO: 41, or a variant thereof modified by one amino acid substitution.

In a thirty-fourth aspect the invention relates to an antibody or antigen binding fragment thereof that specifically binds to Pepsinogen C and competes for binding to Pepsinogen C with the antibody consisting of
(i) a heavy chain variable domain (VH) as shown in SEQ ID NO: 9; and
(ii) a light chain variable domain (VL) as shown in SEQ ID NO: 10.

In one embodiment the antibody or antigen binding fragment thereof according to the invention comprises a VH comprising a CDR-H1 as shown in the amino acid sequence of SEQ ID NO: 11 or a variant thereof modified by one amino acid substitution; a CDR-H2 as shown in the amino acid sequence of SEQ ID NO: 12, or a variant thereof modified by one amino acid substitution; and a CDR-H3 as shown in the amino acid sequence of SEQ ID NO: 13, or a variant thereof modified by one amino acid substitution; and a VL comprising a CDR-L1 as shown in the amino acid sequence of SEQ ID NO: 14 or a variant thereof modified by one amino acid substitution; a CDR-L2 as shown in the amino acid sequence of SEQ ID NO: 15, or a variant thereof modified by one amino acid substitution; and a CDR-L3 as shown in the amino acid sequence of SEQ ID NO: 16, or a variant thereof modified by one amino acid substitution.

In a thirty-fifth aspect the invention relates to a polynucleotide or a set of polynucleotides encoding
(i) the heavy chain variable domain of the antibody or antigen binding fragment thereof according to the invention, and
(ii) the light chain variable domain of the antibody or antigen binding fragment thereof according to the invention.

Further provided herein is a vector that comprises a polynucleotide of the invention.

In a further aspect the present invention relates to a host cell comprising a polynucleotide according to the invention, or a vector according to the invention and expressing the antibody or antigen binding fragment thereof according to the invention.

The host cell may be a prokaryotic cell or a eukaryotic cell. In a preferred embodiment, the host cell is a eukaryotic cell. In a particular embodiment, the cell is a HEK cell. In another particular embodiment the host cell is a CHO cell.

When recombinant expression vectors encoding the heavy and/or light chain of the antibody of the invention as disclosed herein are introduced into host cells, the antibodies or antibody antigen binding fragments are produced by culturing the host cells for a period of time sufficient to allow for expression of the antibody or antigen binding fragment in the host cell or, preferably, to allow for secretion of the antibody or antigen binding fragment into the culture medium in which the host cells are grown. Antibodies and/or antigen binding fragments can be recovered from the culture medium using standard protein purification methods. Methods for purification of antibodies are well known in the art. Exemplary purification methods are described in the appended Examples.

Accordingly, the invention also provides in a further aspect a method for the production of an antibody specifically binding to Pepsinogen C as disclosed herein. The method comprises culturing a host cell of the invention under suitable conditions and isolating the antibody produced. By purification steps, e.g. as described in the appended Examples an isolated antibody of the invention can be derived.

The invention further provides in an aspect an antibody or an antigen binding fragment obtainable by any of the methods disclosed herein.

In a further aspect the invention provides a composition comprising an antibody of the invention, a polynucleotide of the invention, a vector of the invention, or a host cell of the invention. In a preferred embodiment, the composition is a diagnostic composition, i.e. a composition for use in diagnostic applications. In preferred embodiments, the composition is for use in an in vitro diagnostic test for detecting Pepsinogen C. In a preferred embodiment the diagnostic composition may be a reagent for an immunoassay for detecting Pepsinogen C. The diagnostic composition is preferably configured such that it allows for detection of Pepsinogen C in a sample obtained from a subject. The sample is preferably a blood sample (e.g. whole blood, serum or plasma).

In a further aspect the invention provides a composition comprising
a) the antibody or antigen binding fragment thereof according to the thirty-third aspect of the invention or according to any embodiment relating to the thirty-third aspect of the invention and
the antibody or antigen binding fragment thereof according the thirty-fourth aspect of the invention or according to any embodiment relating to the thirty-fourth aspect of the invention.

Also provided is a composition comprising a polynucleotide, a vector or a host cell relating to the antibody of the thirty-third aspect of the invention or embodiments thereof and a polynucleotide, a vector or a host cell relating to the antibody of the thirty-fourth aspect of the invention or embodiments thereof.

In a further aspect the invention relates to the use of the antibody or antigen binding fragment thereof according to the invention, the composition according to the invention, or the kit according to the invention for an in vitro immunoassay, in particular an in vitro immunoassay for detecting Pepsinogen C in a sample. Accordingly, also provided is an antibody of the invention for use in an immunoassay (e.g. a heterogeneous immunoassay) for detecting Pepsinogen C.

In a further aspect, the present invention provides an in vitro method for detecting Pepsinogen C in a sample using the antibody of the invention. The antibody may be part of a composition, in particular a diagnostic composition of the invention.

The sample may be a body fluid, such as a blood sample, cerebrospinal fluid, seminal fluid, saliva or urine. In embodiments, the sample is a blood sample, such as whole blood, serum or plasma. In embodiments, the sample is serum or plasma.

The method for detecting may comprise (a) contacting a sample comprising Pepsinogen C with the antibody, or antigen binding fragment thereof, according to the invention, thereby forming a complex comprising Pepsinogen C and said antibody, or antigen binding fragment thereof, and (b) detecting the complex formed in step (a), thereby detecting Pepsinogen C in said sample.

In a thirty-sixth aspect the invention provides an in vitro method for detecting Pepsinogen C in a sample, comprising the following steps:
1) contacting a sample comprising Pepsinogen C with a first antibody, or antigen binding fragment thereof, and a second antibody, or antigen binding fragment thereof, wherein
(a) the first antibody is
(i) the antibody or antigen binding fragment thereof according to the thirty-third aspect of the invention or to embodiments of the thirty-third aspect; or
(ii) is the antibody or antigen binding fragment thereof according to the thirty-fourth aspect of the invention or to embodiments of the thirty-fourth aspect; and
(b) the second antibody specifically binds to Pepsinogen C and does not compete with the first antibody for specifically binding to Pepsinogen C;
2) incubating the resulting mixture of 1) to form a complex comprising Pepsinogen C and said first antibody or antigen binding fragment thereof and said second antibody or antigen binding fragment thereof, and
3) detecting Pepsinogen C in the sample by detecting the formation of the complex as defined in 2).

In one embodiment the invention relates to the kit according to the thirty-second aspect, wherein the first antibody or antigen binding fragment comprises
(a) a VH comprising a CDR-H1 as shown in the amino acid sequence of SEQ ID NO: 3 or a variant thereof modified by one amino acid substitution; a CDR-H2 as shown in the amino acid sequence of SEQ ID NO: 4, or a variant thereof modified by one amino acid substitution; and a CDR-H3 as shown in the amino acid sequence of SEQ ID NO: 5, or a variant thereof modified by one amino acid substitution; and
a VL comprising a CDR-L1 as shown in the amino acid sequence of SEQ ID NO: 6 or a variant thereof modified by one amino acid substitution; a CDR-L2 as shown in the amino acid sequence of SEQ ID NO: 7, or a variant thereof modified by one amino acid substitution; and a CDR-L3 as shown in the amino acid sequence of SEQ ID NO: 52, or a variant thereof modified by one amino acid substitution; or
(b) a VH comprising a CDR-H1 as shown in the amino acid sequence of SEQ ID NO: 36 or a variant thereof modified by one amino acid substitution; a CDR-H2 as shown in the amino acid sequence of SEQ ID NO: 37, or a variant thereof modified by one amino acid substitution; and a CDR-H3 as shown in the amino acid sequence of SEQ ID NO: 38, or a variant thereof modified by one amino acid substitution; and
a VL comprising a CDR-L1 as shown in the amino acid sequence of SEQ ID NO: 39 or a variant thereof modified by one amino acid substitution; a CDR-L2 as shown in the amino acid sequence of SEQ ID NO: 40, or a variant thereof modified by one amino acid substitution; and a CDR-L3 as shown in the amino acid sequence of SEQ ID NO: 41, or a variant thereof modified by one amino acid substitution.

In one embodiment the invention provides the kit according to the thirty-second aspect of the invention, wherein the second antibody or antigen binding fragment thereof comprises
a VH comprising a CDR-H1 as shown in the amino acid sequence of SEQ ID NO: 11 or a variant thereof modified by one amino acid substitution; a CDR-H2 as shown in the amino acid sequence of SEQ ID NO: 12, or a variant thereof modified by one amino acid substitution; and a CDR-H3 as shown in the amino acid sequence of SEQ ID NO: 13, or a variant thereof modified by one amino acid substitution; and
VL comprising a CDR-L1 as shown in the amino acid sequence of SEQ ID NO: 14 or a variant thereof modified by one amino acid substitution; a CDR-L2 as shown in the amino acid sequence of SEQ ID NO: 15, or a variant thereof modified by one amino acid substitution; and a CDR-L3 as shown in the amino acid sequence of SEQ ID NO: 16, or a variant thereof modified by one amino acid substitution.

In one embodiment the invention provides the kit according to the thirty-second aspect of the invention, wherein the first antibody or antigen binding fragment comprises
(a) a VH comprising a CDR-H1 as shown in the amino acid sequence of SEQ ID NO: 3 or a variant thereof modified by one amino acid substitution; a CDR-H2 as shown in the amino acid sequence of SEQ ID NO: 4, or a variant thereof modified by one amino acid substitution; and a CDR-H3 as shown in the amino acid sequence of SEQ ID NO: 5, or a variant thereof modified by one amino acid substitution; and
a VL comprising a CDR-L1 as shown in the amino acid sequence of SEQ ID NO: 6 or a variant thereof modified by one amino acid substitution; a CDR-L2 as shown in the amino acid sequence of SEQ ID NO: 7, or a variant thereof modified by one amino acid substitution; and a CDR-L3 as shown in the amino acid sequence of SEQ ID NO: 52, or a variant thereof modified by one amino acid substitution; or
(b) a VH comprising a CDR-H1 as shown in the amino acid sequence of SEQ ID NO: 36 or a variant thereof modified by one amino acid substitution; a CDR-H2 as shown in the amino acid sequence of SEQ ID NO: 37, or a variant thereof modified by one amino acid substitution; and a CDR-H3 as shown in the amino acid sequence of SEQ ID NO: 38, or a variant thereof modified by one amino acid substitution; and
a VL comprising a CDR-L1 as shown in the amino acid sequence of SEQ ID NO: 39 or a variant thereof modified by one amino acid substitution; a CDR-L2 as shown in the amino acid sequence of SEQ ID NO: 40, or a variant thereof modified by one amino acid substitution; and a CDR-L3 as shown in the amino acid sequence of SEQ ID NO: 41, or a variant thereof modified by one amino acid substitution; and
wherein the second antibody, or antigen binding fragment thereof, comprises
(c) a VH comprising a CDR-H1 as shown in the amino acid sequence of SEQ ID NO: 11 or a variant thereof modified by one amino acid substitution; a CDR-H2 as shown in the amino acid sequence of SEQ ID NO: 12, or a variant thereof modified by one amino acid substitution; and a CDR-H3 as shown in the amino acid sequence of SEQ ID NO: 13, or a variant thereof modified by one amino acid substitution; and
VL comprising a CDR-L1 as shown in the amino acid sequence of SEQ ID NO: 14 or a variant thereof modified by one amino acid substitution; a CDR-L2 as shown in the amino acid sequence of SEQ ID NO: 15, or a variant thereof modified by one amino acid substitution; and a CDR-L3 as shown in the amino acid sequence of SEQ ID NO: 16, or a variant thereof modified by one amino acid substitution.

In a further aspect the invention provides an in vitro method for detecting Pepsinogen C in a sample, comprising the following steps:
1) contacting a sample comprising Pepsinogen C with a first antibody, or antigen binding fragment thereof, and a second antibody, or antigen binding fragment thereof, wherein
(a) the first antibody is
(i) the antibody or antigen binding fragment thereof according to the first aspect of the invention or to embodiments of the first aspect; or
(ii) is the antibody or antigen binding fragment thereof according to the second aspect of the invention or to embodiments of the second aspect; and
(b) the second antibody specifically binds to Pepsinogen C and does not compete with the first antibody for specifically binding to Pepsinogen C;
2) incubating the resulting mixture of 1) to form a complex comprising Pepsinogen C and said first antibody or antigen binding fragment thereof and said second antibody or antigen binding fragment thereof, and
3) detecting Pepsinogen C in the sample by detecting the formation of the complex as defined in 2), and
(c) optionally quantifying the amount of Pepsinogen C in the sample.

In a further aspect the invention provides an in vitro method for detecting Pepsinogen C in a sample, comprising the following steps:
1) contacting a sample comprising Pepsinogen C with a first antibody, or antigen binding fragment thereof, and a second antibody, or antigen binding fragment thereof, wherein
(a) the first antibody is
   (i) the antibody or antigen binding fragment thereof according to the sixteenth aspect of the invention or to embodiments of the sixteenth aspect; or
   (ii) the antibody or antigen binding fragment thereof according to the nineteenth aspect of the invention or to embodiments of the nineteenth aspect; and
(b) the second antibody does not compete with the first antibody for specifically binding to Pepsinogen C; and
2) incubating the resulting mixture of 1) to form a complex comprising Pepsinogen C and said first antibody or antigen binding fragment thereof and said second antibody or antigen binding fragment thereof; and
3) detecting Pepsinogen C in the sample by detecting the formation of the complex as defined in 2), and
(c) optionally quantifying the amount of Pepsinogen C in the sample.

In a further aspect, the present invention provides for an in vitro method for detecting and/or quantifying Pepsinogen C in a sample, comprising
(a) contacting a sample comprising Pepsinogen C with the antibody, or antigen binding fragment thereof, according to the invention, thereby forming a complex comprising Pepsinogen C and said antibody, or antigen binding fragment thereof, and
(b) detecting the complex formed in step (a), thereby detecting Pepsinogen C in said sample, and
(c) optionally quantifying the amount of Pepsinogen C in said sample.

The present invention in particular also relates to the following items:

1. An antibody or antigen binding fragment thereof that specifically binds to Pepsinogen C and competes for binding to Pepsinogen C with the antibody consisting of
(i) a heavy chain variable domain (VH) as shown in SEQ ID NO: 1; and
(ii) a light chain variable domain (VL) as shown in SEQ ID NO: 2, preferable wherein the antibody or antigen binding fragment thereof binds the same epitope as the antibody consisting of a heavy chain variable domain (VH) as shown in SEQ ID NO: 1; and a light chain variable domain (VL) as shown in SEQ ID NO: 2.

2. The antibody or antigen binding fragment thereof according to item 1, comprising
(a) a VH comprising a CDR-H1 as shown in the amino acid sequence of SEQ ID NO: 3 or a variant thereof modified by one amino acid substitution; a CDR-H2 as shown in the amino acid sequence of SEQ ID NO: 4, or a variant thereof modified by one amino acid substitution; and a CDR-H3 as shown in the amino acid sequence of SEQ ID NO: 5, or a variant thereof modified by one amino acid substitution; and
a VL comprising a CDR-L1 as shown in the amino acid sequence of SEQ ID NO: 6 or a variant thereof modified by one amino acid substitution; a CDR-L2 as shown in the amino acid sequence of SEQ ID NO: 7, or a variant thereof modified by one amino acid substitution; and a CDR-L3 as shown in the amino acid sequence of SEQ ID NO: 52, or a variant thereof modified by one amino acid substitution; or
(b) a VH comprising a CDR-H1 as shown in the amino acid sequence of SEQ ID NO: 36 or a variant thereof modified by one amino acid substitution; a CDR-H2 as shown in the amino acid sequence of SEQ ID NO: 37, or a variant thereof modified by one amino acid substitution; and a CDR-H3 as shown in the amino acid sequence of SEQ ID NO: 38, or a variant thereof modified by one amino acid substitution; and
a VL comprising a CDR-L1 as shown in the amino acid sequence of SEQ ID NO: 39 or a variant thereof modified by one amino acid substitution; a CDR-L2 as shown in the amino acid sequence of SEQ ID NO: 40, or a variant thereof modified by one amino acid substitution; and a CDR-L3 as shown in the amino acid sequence of SEQ ID NO: 41, or a variant thereof modified by one amino acid substitution.

3. The antibody or antigen binding fragment thereof according to item 2, wherein all the amino acid substitutions are conservative amino acid substitutions.

4. The antibody or antigen binding fragment thereof according to item 3, wherein each of said said conservative amino acid substitutions is the substitution of an amino acid with another amino acid selected from its same group, wherein the groups of amino acids are
a) the nonpolar, hydrophobic amino acids consisting of Gly, Ala, Val, Leu, Ile, Phe, Try, Trp, and Met;
b) the polar, neutral amino acids consisting of Ser, Thr, Asn, and Gln;
c) the positively charged, basic amino acids consisting of Arg, Lys, and His, and
d) the negatively charged, amino acids consisting of Asp and Glu
wherein if Cys is to be conservatively substituted, it is substituted with Ser or Ala, and
wherein if Pro is to be conservatively substituted it is substituted with Ala.

5. The antibody or antigen binding fragment thereof according to item 3 or 4, wherein each of said conservative amino acid substitutions is a highly conservative amino acid substitution selected from
a) substitution of Ala with Val, Leu, Ile or Gly;
b) substitution of Arg with Lys;
c) substitution of Asn with Gln;
d) substitution of Asp with Glu;
e) substitution of Cys with Ser;
f) substitution of Gln with Asn;
g) substitution of Glu with Asp;
h) substitution of Gly with Ala;
i) substitution of His with Arg;
j) substitution of Ile with Leu, Val or Ala;
k) substitution of Leu with Ile, Val or Ala;
l) substitution of Lys with Arg;
m) substitution of Met with Leu, Ile or Val;
n) substitution of Phe with Try or Trp;
o) substitution of Pro with Ala;
p) substitution of Ser with Thr;
q) substitution of Thr with Ser;
r) substitution of Trp with Phe or Tyr;
s) substitution of Tyr with Phe or Trp; and
t) substitution of Val with Leu, Ile or Ala.

6. The antibody or antigen binding fragment thereof according to any one of items 1 to 4, comprising
(a) a VH comprising a CDR-H1 as shown in the amino acid sequence of SEQ ID NO: 3; a CDR-H2 as shown in the amino acid sequence of SEQ ID NO: 4; and a CDR-H3 as shown in the amino acid sequence of SEQ ID NO: 5; and
a VL comprising a CDR-L1 as shown in the amino acid sequence of SEQ ID NO: 6; a CDR-L2 as shown in the amino acid sequence of SEQ ID NO: 7; and a CDR-L3 as shown in the amino acid sequence of SEQ ID NO: 52; or
(b) a VH comprising a CDR-H1 as shown in the amino acid sequence of SEQ ID NO: 36; a CDR-H2 as shown in the amino acid sequence of SEQ ID NO: 37; and a CDR-H3 as shown in the amino acid sequence of SEQ ID NO: 38; and
a VL comprising a CDR-L1 as shown in the amino acid sequence of SEQ ID NO: 39; a CDR-L2 as shown in the amino acid sequence of SEQ ID NO: 40; and a CDR-L3 as shown in the amino acid sequence of SEQ ID NO: 41.

7. The antibody or antigen binding fragment thereof according to item 6 (a), wherein the VL comprising a CDR-L3 as shown in SEQ ID NO: 8 or SEQ ID NO: 57.

8. The antibody or antigen binding fragment thereof according to any one of items 1 to 7, wherein the antibody comprises a VL consisting of framework regions (FW) and CDRs as represented in formula I:

(FW-L1)-(CDR-L1)-(FW-L2)-(CDR-L2)-(FW-L3)-(CDR-L3)-(FW-L4) (formula I)
and a VH consisting of FWs and CDRs as represented in formula II:

(FW-H1)-(CDR-H1)-(FW-H2)-(CDR-H2)-(FW-H3)-(CDR-H3)-(FW-H4) (formula II),
wherein the VH and VL comprise the sequences according to (a) and the FWs comprise the following amino acid sequences or a variant thereof that is at least 85% homologous thereto:
FW-L1 the amino acid sequence of SEQ ID NO: 18;
FW-L2 the amino acid sequence of SEQ ID NO: 56;
FW-L3 the amino acid sequence of SEQ ID NO: 20;
FW-L4 the amino acid sequence of SEQ ID NO: 21;
FW-H1 the amino acid sequence of SEQ ID NO: 53;
FW-H2 the amino acid sequence of SEQ ID NO: 54;
FW-H3 the amino acid sequence of SEQ ID NO: 55;
FW-H4 the amino acid sequence of SEQ ID NO: 25; or
wherein the VH and VL comprise the sequences according to (b) and the FWs comprise the following amino acid sequences or a variant thereof that is at least 85% homologous thereto:
FW-L1 the amino acid sequence of SEQ ID NO: 42;
FW-L2 the amino acid sequence of SEQ ID NO: 43;
FW-L3 the amino acid sequence of SEQ ID NO: 44;
FW-L4 the amino acid sequence of SEQ ID NO: 45;
FW-H1 the amino acid sequence of SEQ ID NO: 46;
FW-H2 the amino acid sequence of SEQ ID NO: 47;
FW-H3 the amino acid sequence of SEQ ID NO: 48;
FW-H4 the amino acid sequence of SEQ ID NO: 49.

9. The antibody or antigen binding fragment thereof according to any one of items 1 to 8, wherein the antibody comprises a VL consisting of framework regions (FW) and CDRs as represented in formula I:

(FW-L1)-(CDR-L1)-(FW-L2)-(CDR-L2)-(FW-L3)-(CDR-L3)-(FW-L4) (formula I)
and a VH consisting of FWs and CDRs as represented in formula II:

(FW-H1)-(CDR-H1)-(FW-H2)-(CDR-H2)-(FW-H3)-(CDR-H3)-(FW-H4) (formula II),
wherein the VH and VL comprise the sequences according to (a) and the FWs comprise the following amino acid sequences or a variant thereof modified by at most three amino acid substitutions:
FW-L1 the amino acid sequence of SEQ ID NO: 18;
FW-L2 the amino acid sequence of SEQ ID NO: 56;
FW-L3 the amino acid sequence of SEQ ID NO: 20;
FW-L4 the amino acid sequence of SEQ ID NO: 21;
FW-H1 the amino acid sequence of SEQ ID NO: 53;
FW-H2 the amino acid sequence of SEQ ID NO: 54;
FW-H3 the amino acid sequence of SEQ ID NO: 55;
FW-H4 the amino acid sequence of SEQ ID NO: 25; or
wherein the VH and VL comprise the sequences according to (b) and the FWs comprise the following amino acid sequences or a variant thereof modified by at most three amino acid substitutions:
FW-L1 the amino acid sequence of SEQ ID NO: 42;
FW-L2 the amino acid sequence of SEQ ID NO: 43;
FW-L3 the amino acid sequence of SEQ ID NO: 44;
FW-L4 the amino acid sequence of SEQ ID NO: 45;
FW-H1 the amino acid sequence of SEQ ID NO: 46;
FW-H2 the amino acid sequence of SEQ ID NO: 47;
FW-H3 the amino acid sequence of SEQ ID NO: 48;
FW-H4 the amino acid sequence of SEQ ID NO: 49.

10. The antibody or antigen binding fragment thereof according to item 9, wherein all the amino acid substitutions are conservative amino acid substitutions.

11. The antibody or antigen binding fragment thereof according to item 10, wherein each of said conservative amino acid substitutions is the substitution of an amino acid with another amino acid selected from its same group, wherein the groups of amino acids are
a) the nonpolar, hydrophobic amino acids consisting of Gly, Ala, Val, Leu, Ile, Phe, Try, Trp, and Met;
b) the polar, neutral amino acids consisting of Ser, Thr, Asn, and Gln;
c) the positively charged, basic amino acids consisting of Arg, Lys, and His, and
d) the negatively charged, amino acids consisting of Asp and Glu
wherein if Cys is to be conservatively substituted, it is substituted with Ser or Ala, and
wherein if Pro is to be conservatively substituted it is substituted with Ala.

12. The antibody or antigen binding fragment thereof according to item 10 or 11, wherein each of said conservative amino acid substitutions is a highly conservative amino acid substitution selected from
a) substitution of Ala with Val, Leu, Ile or Gly;
b) substitution of Arg with Lys;
c) substitution of Asn with Gln;
d) substitution of Asp with Glu;
e) substitution of Cys with Ser;
f) substitution of Gln with Asn;
g) substitution of Glu with Asp;
h) substitution of Gly with Ala;
i) substitution of His with Arg;
j) substitution of Ile with Leu, Val or Ala;
k) substitution of Leu with Ile, Val or Ala;
l) substitution of Lys with Arg;
m) substitution of Met with Leu, Ile or Val;
n) substitution of Phe with Try or Trp;
o) substitution of Pro with Ala;
p) substitution of Ser with Thr;
q) substitution of Thr with Ser;
r) substitution of Trp with Phe or Tyr;
s) substitution of Tyr with Phe or Trp; and
t) substitution of Val with Leu, Ile or Ala.

13. The antibody or antigen binding fragment thereof according to any one of items 1 to 12, comprising
(i) a VH comprising a sequence as shown in SEQ ID NO: 1; and/or
(ii) a VL comprising a sequence as shown in SEQ ID NO: 2.

14. The antibody or antigen binding fragment thereof according to any one of items 1 to 12,
comprising
(i) a VH comprising a sequence as shown in SEQ ID NO: 58; and/or
(ii) a VL comprising a sequence as shown in SEQ ID NO: 59.

15. The antibody or antigen binding fragment thereof according to any one of items 1 to 12,
comprising
(i) a VH comprising a sequence as shown in SEQ ID NO: 60; and/or
(ii) a VL comprising a sequence as shown in SEQ ID NO: 61.

16. The antibody or antigen binding fragment thereof according to any one of items 1 to 12, comprising
(i) a VH comprising a sequence as shown in SEQ ID NO: 34; and/or
(ii) a VL comprising a sequence as shown in SEQ ID NO: 35.

17. An antibody or antigen binding fragment thereof that specifically binds to Pepsinogen C and competes for binding to Pepsinogen C with the antibody consisting of
(i) a heavy chain variable domain (VH) as shown in SEQ ID NO: 9; and
(ii) a light chain variable domain (VL) as shown in SEQ ID NO: 10, preferable wherein the antibody or antigen binding fragment thereof binds the same epitope as the antibody consisting of a heavy chain variable domain (VH) as shown in SEQ ID NO: 9; and a light chain variable domain (VL) as shown in SEQ ID NO: 10.

18. The antibody or antigen binding fragment thereof according to item 17, comprising
a VH comprising a CDR-H1 as shown in the amino acid sequence of SEQ ID NO: 11 or a variant thereof modified by one amino acid substitution; a CDR-H2 as shown in the amino acid sequence of SEQ ID NO: 12, or a variant thereof modified by one amino acid substitution; and a CDR-H3 as shown in the amino acid sequence of SEQ ID NO: 13, or a variant thereof modified by one amino acid substitution; and
a VL comprising a CDR-L1 as shown in the amino acid sequence of SEQ ID NO: 14 or a variant thereof modified by one amino acid substitution; a CDR-L2 as shown in the amino acid sequence of SEQ ID NO: 15, or a variant thereof modified by one amino acid substitution; and a CDR-L3 as shown in the amino acid sequence of SEQ ID NO: 16, or a variant thereof modified by one amino acid substitution.

19. The antibody or antigen binding fragment thereof according to item 18, wherein all the amino acid substitutions are conservative amino acid substitutions.

20. The antibody or antigen binding fragment thereof according to item 18 or 19, wherein each of said conservative amino acid substitutions is the substitution of an amino acid with another amino acid selected from its same group, wherein the groups of amino acids are
a) the nonpolar, hydrophobic amino acids consisting of Gly, Ala, Val, Leu, Ile, Phe, Try, Trp, and Met;
b) the polar, neutral amino acids consisting of Ser, Thr, Asn, and Gln;
c) the positively charged, basic amino acids consisting of Arg, Lys, and His, and
d) the negatively charged, amino acids consisting of Asp and Glu
wherein if Cys is to be conservatively substituted, it is substituted with Ser or Ala, and
wherein if Pro is to be conservatively substituted it is substituted with Ala.

21. The antibody or antigen binding fragment thereof according to item 19 or 20, wherein each of said conservative amino acid substitutions is a highly conservative amino acid substitution selected from
a) substitution of Ala with Val, Leu, Ile or Gly;
b) substitution of Arg with Lys;
c) substitution of Asn with Gln;
d) substitution of Asp with Glu;
e) substitution of Cys with Ser;
f) substitution of Gln with Asn;
g) substitution of Glu with Asp;
h) substitution of Gly with Ala;
i) substitution of His with Arg;
j) substitution of Ile with Leu, Val or Ala;
k) substitution of Leu with Ile, Val or Ala;
l) substitution of Lys with Arg;
m) substitution of Met with Leu, Ile or Val;
n) substitution of Phe with Try or Trp;
o) substitution of Pro with Ala;
p) substitution of Ser with Thr;
q) substitution of Thr with Ser;
r) substitution of Trp with Phe or Tyr;
s) substitution of Tyr with Phe or Trp; and
t) substitution of Val with Leu, Ile or Ala.

22. The antibody or antigen binding fragment thereof according to any one of items 17 to 21, comprising
a VH comprising a CDR-H1 as shown in the amino acid sequence of SEQ ID NO: 11; a CDR-H2 as shown in the amino acid sequence of SEQ ID NO: 12; and a CDR-H3 as shown in the amino acid sequence of SEQ ID NO: 13; and
a VL comprising a CDR-L1 as shown in the amino acid sequence of SEQ ID NO: 14; a CDR-L2 as shown in the amino acid sequence of SEQ ID NO: 15; and a CDR-L3 as shown in the amino acid sequence of SEQ ID NO: 16.

23. The antibody or antigen binding fragment thereof according to any one of items 17 to 22, wherein the antibody comprises a VL consisting of framework regions (FW) and CDRs as represented in formula I:

(FW-L1)-(CDR-L1)-(FW-L2)-(CDR-L2)-(FW-L3)-(CDR-L3)-(FW-L4) (formula I)
and a VH consisting of FWs and CDRs as represented in formula II:

(FW-H1)-(CDR-H1)-(FW-H2)-(CDR-H2)-(FW-H3)-(CDR-H3)-(FW-H4) (formula II),
wherein the FWs comprise the following amino acid sequences or a variant thereof that is at least 85% homologous thereto:
FW-L1 the amino acid sequence of SEQ ID NO: 26;
FW-L2 the amino acid sequence of SEQ ID NO: 27;
FW-L3 the amino acid sequence of SEQ ID NO: 28;
FW-L4 the amino acid sequence of SEQ ID NO: 29;
FW-H1 the amino acid sequence of SEQ ID NO: 30;
FW-H2 the amino acid sequence of SEQ ID NO: 31;
FW-H3 the amino acid sequence of SEQ ID NO: 32;
FW-H4 the amino acid sequence of SEQ ID NO: 33.

24. The antibody or antigen binding fragment thereof according to any one of items 17 to 23, wherein the antibody comprises a VL consisting of framework regions (FW) and CDRs as represented in formula I:

(FW-L1)-(CDR-L1)-(FW-L2)-(CDR-L2)-(FW-L3)-(CDR-L3)-(FW-L4) (formula I)
and a VH consisting of FWs and CDRs as represented in formula II:

(FW-H1)-(CDR-H1)-(FW-H2)-(CDR-H2)-(FW-H3)-(CDR-H3)-(FW-H4) (formula II),
wherein the FWs comprise the following amino acid sequences or a variant thereof modified by at most three amino acid substitutions:
FW-L1 the amino acid sequence of SEQ ID NO: 26;
FW-L2 the amino acid sequence of SEQ ID NO: 27;
FW-L3 the amino acid sequence of SEQ ID NO: 28;
FW-L4 the amino acid sequence of SEQ ID NO: 29;
FW-H1 the amino acid sequence of SEQ ID NO: 30;
FW-H2 the amino acid sequence of SEQ ID NO: 31;
FW-H3 the amino acid sequence of SEQ ID NO: 32;
FW-H4 the amino acid sequence of SEQ ID NO: 33.

25. The antibody or antigen binding fragment thereof according to item 24, wherein all the amino acid substitutions are conservative amino acid substitutions.

26. The antibody or antigen binding fragment thereof according to item 25, wherein each of said conservative amino acid substitutions is the substitution of an amino acid with another amino acid selected from its same group, wherein the groups of amino acids are
a) the nonpolar, hydrophobic amino acids consisting of Gly, Ala, Val, Leu, Ile, Phe, Try, Trp, and Met;
b) the polar, neutral amino acids consisting of Ser, Thr, Asn, and Gln;
c) the positively charged, basic amino acids consisting of Arg, Lys, and His, and
d) the negatively charged, amino acids consisting of Asp and Glu
wherein if Cys is to be conservatively substituted, it is substituted with Ser or Ala, and
wherein if Pro is to be conservatively substituted it is substituted with Ala.

27. The antibody or antigen binding fragment thereof according to item 25 or 26, wherein each of said conservative amino acid substitutions is a highly conservative amino acid substitution selected from
a) substitution of Ala with Val, Leu, Ile or Gly;
b) substitution of Arg with Lys;
c) substitution of Asn with Gln;
d) substitution of Asp with Glu;
e) substitution of Cys with Ser;
f) substitution of Gln with Asn;
g) substitution of Glu with Asp;
h) substitution of Gly with Ala;
i) substitution of His with Arg;
j) substitution of Ile with Leu, Val or Ala;
k) substitution of Leu with Ile, Val or Ala;
l) substitution of Lys with Arg;
m) substitution of Met with Leu, Ile or Val;
n) substitution of Phe with Try or Trp;
o) substitution of Pro with Ala;
p) substitution of Ser with Thr;
q) substitution of Thr with Ser;
r) substitution of Trp with Phe or Tyr;
s) substitution of Tyr with Phe or Trp; and
t) substitution of Val with Leu, Ile or Ala.

28. The antibody or antigen binding fragment thereof according to any one of items 17 to 27, comprising
(i) a VH comprising a sequence as shown in SEQ ID NO: 9; and/or
(ii) a VL comprising a sequence as shown in SEQ ID NO: 10.

29. The antibody or antigen binding fragment thereof according to any one of items 1 to 27, wherein the antibody is a monoclonal antibody.

30. The antibody or antigen binding fragment thereof according to any one of items 1 to 29, wherein the Pepsinogen C comprises SEQ ID NO: 17, preferably wherein the Pepsinogen C consists of SEQ ID NO: 17.

31. The antibody or antigen binding fragment thereof according to any one of items 1 to 30, wherein the antibody or antigen binding fragment thereof binds to Pepsinogen C consisting of SEQ ID NO: 62 with a KD of 2 nM or less, or 1.5 nM or less, or 1 nM or less, or 0.6 nM or less, preferably wherein the K_{D} is measured at 37 °C.

32. The antibody or antigen binding fragment thereof according to any one of items 1 to 31, wherein the antibody or antigen binding fragment thereof binds to Pepsinogen C consisting of SEQ ID NO: 62 with t/2-diss at 37 °C of 20 minutes or longer, preferably 30 minutes or longer.

33. The antibody or antigen binding fragment thereof according to any one of items 1 to 32, wherein the association rate kₐ for Pepsinogen C comprising SEQ ID NO: 62 is at least 10⁵ M⁻¹s⁻¹, preferable at least 2.5×10⁵ M⁻¹s⁻¹.

34. The antibody or antigen binding fragment thereof according to any one of items 31 to 33, wherein said K_{D} and/or said kₐ is determined by surface plasmon resonance spectroscopy.

35. The antibody or antigen binding fragment thereof according to item 34, wherein said surface plasmon resonance spectroscopy comprises attaching or capturing the antibody, or antigen binding fragment thereof, on a CM5 sensor chip and injecting Pepsinogen C comprising SEQ ID NO: 62 as analyte, wherein said determination is conducted at a temperature of 37 °C using HBS-ET buffer (10 mM HEPES, 150 mM NaCl, 3 mM EDTA, 0.05% (w/v) Tween 20®).

36. The antibody or antigen binding fragment thereof according to any one of items 34 to 35, wherein the surface plasmon resonance spectroscopy is performed with a Biacore 8 k or B4000 instrument.

37. The antibody or antigen binding fragment thereof according to any one of items 34 to 36, wherein the determination of said KD and/or said ka comprises fitting the surface plasmon resonance data using a Langmuir fitting model.

38. The antibody or antigen binding fragment thereof according to any one of items 1 to 37, wherein the antibody does not specifically bind to Pepsinogen A.

39. A polynucleotide or a set of polynucleotides encoding
(i) the heavy chain variable domain of the antibody or antigen binding fragment thereof according to any one of items 1 to 38, and/or
(ii) the light chain variable domain of the antibody or antigen binding fragment thereof according to any one of items 1 to 38.

40. A vector comprising the polynucleotide or the set of polynucleotides according to item 39.

41. A host cell comprising the polynucleotide or the set of polynucleotides according to item 39, or the vector according to item 40 and expressing the antibody, or antigen binding fragment thereof, according to any one of items 1 to 38.

42. A host cell according to item 41, wherein the host cell is a prokaryotic cell or a eukaryotic cell.

43. A host cell according to item 42, wherein the host cell is a eukaryotic cell, wherein said host cell is a HEK293 cell or a CHO cell.

44. A method of producing the antibody or antigen binding fragment thereof according to any one of items 1 to 38 comprising culturing the host cell according to any one of items 41 to 43 and isolating said antibody or antigen binding fragment thereof.

45. An antibody, or an antigen binding fragment thereof, according to any one of items 1 to 38 obtainable by the method of item 44.

46. A composition comprising the antibody, or antigen binding fragment thereof, according to any one of items 1 to 38, the polynucleotide or set of polynucleotides according to item 39, the vector according to item 40, or the host cell according to any one of items 41 to 43.

47. A composition comprising
a) the antibody or antigen binding fragment thereof according to any one of items 1-16 or 45 or according to any one of items 29 to 38 as dependent from any one of items 1 to 16 and
the antibody or antigen binding fragment thereof according to any one of items 17-28 or 45 or according to any one of items 29 to 38 as dependent from any one of items 17 to 28;
b) the polynucleotide or set of polynucleotides of item 39 as dependent from any one of items 1-16 or 45 or according to any one of items 29 to 38 as dependent from any one of items 1 to 16; and
the polynucleotide or set of polynucleotides of item 39 as dependent from any one of items 17-28 or 45 or according to any one of items 29 to 38 as dependent from any one of items 17 to 28;
c) the vector of item 40 as dependent from any one of items 1-16 or 45 or according to any one of items 29 to 38 as dependent from any one of items 1 to 16; and
the vector of item 40 as dependent from any one of items 17-28 or 45 or according to any one of items 29 to 38 as dependent from any one of items 17 to 28; or
d) the host cell according to any one of items 41 to 43 as dependent from any one of items 1-16 or 45 or according to any one of items 29 to 38 as dependent from any one of items 1 to 16; and
the host cell according to any one of items 41 to 43 as dependent from any one of items 17-28 or 45 or according to any one of items 29 to 38 as dependent from any one of items 17 to 28.

48. A composition comprising the antibody or antigen binding fragment thereof according to any one of items 1-38 or 45, wherein the composition is a diagnostic composition.

49. A composition comprising
the antibody or antigen binding fragment thereof according to any one of items 1-16 or 45 or according to any one of items 29 to 38 as dependent from any one of items 1 to 16; and
the antibody or antigen binding fragment thereof according to according any one of items 17-28 or 45 or according to any one of items 29 to 38 as dependent from any one of items 17 to 28;
wherein the composition is a diagnostic composition.

50. Use of the antibody or antigen binding fragment thereof according to any one of items 1-38 or 45 or the composition according to item 48 or 49 for an in vitro immunoassay, in particular an in vitro immunoassay for detecting Pepsinogen C in a sample.

51. Use of the antibody, or antigen binding fragment thereof, according to item 50, wherein the immunoassay is a heterogeneous immunoassay.

52. Use of the antibody, or antigen binding fragment thereof, according to item 50 or 51, wherein the sample for said immunoassay is a sample prepared from blood, plasma, or serum.

53. An in vitro method for detecting Pepsinogen C in a sample, comprising
(a) contacting a sample comprising Pepsinogen C with the antibody, or antigen binding fragment thereof, according to any one of items 1-38 or 45, thereby forming a complex comprising Pepsinogen C and said antibody, or antigen binding fragment thereof, and
(b) detecting the complex formed in step (a), thereby detecting Pepsinogen C in said sample.

54. The method according to item 53, wherein the method is a serum immunoassay, wherein the sample is a body fluid, preferably whole blood, serum or plasma.

55. The method according to item 53 to 54, wherein the antibody, or antigen binding fragment thereof, has a detection label attached thereto.

56. The method according to item 55, wherein the detection label is selected from an enzyme, a label emitting light, preferable fluorescence, luminescence, chemiluminescence, electrochemiluminescence, or radioactivity.

57. An in vitro method for detecting Pepsinogen C in a sample, comprising the following steps:
1) contacting a sample comprising Pepsinogen C with a first antibody, or antigen binding fragment thereof, and a second antibody, or antigen binding fragment thereof, wherein
(a) the first antibody is
(i) the antibody or antigen binding fragment thereof according to any one of items 1-16 or 45 or according to any one of items 29 to 38 as dependent from any one of items 1 to 16; or
(ii) the antibody or antigen binding fragment thereof according to according any one of items 17-28 or 45 or according to any one of items 29 to 38 as dependent from any one of items 17 to 28; and
(b) the second antibody specifically binds to Pepsinogen C and does not compete with the first antibody for specifically binding to Pepsinogen C;
2) incubating the resulting mixture of 1) to form a complex comprising Pepsinogen C and said first antibody or antigen binding fragment thereof and said second antibody or antigen binding fragment thereof, and
3) detecting Pepsinogen C in the sample by detecting the formation of the complex as defined in 2).

58. The method according to item 57, wherein (c) one of the first and the second antibody comprises a detection label.

59. The method according to item 58, wherein the detection label is selected from an enzyme, a label emitting light, preferable fluorescence, luminescence, chemiluminescence, electrochemiluminescence, or radioactivity.

60. The method according to item 59, wherein the first antibody or antigen binding fragment thereof or the second antibody or antigen binding fragment thereof not comprising the detection label, comprises a capture label.

61. The method according to item 60, wherein the method further comprises adding to the mixture of (1) particles capable of binding to the capture label and 2.1) separating the particles from the mixture before detecting a signal provided by the detection label.

62. The method according to any one of items 57 to 61, wherein
when the first antibody, or antigen binding fragment thereof, is selected from (i) than the second antibody is selected from (ii); or
when the first antibody, or antigen binding fragment thereof, is selected from (ii) than the second antibody is selected from (i).

63. The method according to any one of items 57 to 62, wherein the method is a serum immunoassay, wherein the sample is a body fluid, preferably whole blood, serum or plasma.

64. The method according to any one of items 57 to 63, wherein the Limit of Quantification (LoQ) is 0.353 ng/mL at 20% CV.

65. The method according to any one of items 57 to 64, wherein the detection of Pepsinogen C in the sample is achieved in 18 minutes or less.

66. A kit comprising the antibody, or the antigen binding fragment thereof, according to any one of items 1-38 or 45.

67. A kit comprising
1) the composition according to item 49, or
2) a first antibody or antigen binding fragment thereof and a second antibody or antigen binding fragment thereof wherein one of the first or second antibody or antigen binding fragment thereof is
a) the antibody or antigen binding fragment thereof according to any one of items 1-16 or 45 or according to any one of items 29 to 38 as dependent from any one of items 1 to 16;
and the other of the first or second antibody or antigen binding fragment thereof is
b) the antibody or antigen binding fragment thereof according to any one of items 17-28 or 45 or according to any one of items 29 to 38 as dependent from any one of items 17 to 28.

68. The kit according to item 67, wherein one of the first and the second antibody or antigen binding fragment thereof comprises a detection label.

69. The kit according to item 68, wherein the first antibody or antigen binding fragment thereof or the second antibody or antigen binding fragment thereof not comprising the detection label, comprises a capture label.

70. The kit according to any one of items 66 to 69, wherein the kit is for detecting Pepsinogen C.

71. The kit according to any one of items 66 to 70 that is an immunoassay kit.

72. An antibody or antigen binding fragment thereof that specifically binds to Pepsinogen C, comprising
(i) a heavy chain variable domain (VH) that is at least 80% homologous to the VH as shown in SEQ ID NO: 50; and
(ii) a light chain variable domain (VL) that is at least 80% homologous to the VL as shown in SEQ ID NO: 51.

73. An antibody or antigen binding fragment thereof that specifically binds to Pepsinogen C, comprising
(i) a VH that is at least 85% homologous to the VH as shown in SEQ ID NO: 50; and/or
(ii) a VL that is at least 85% homologous to the VL as shown in SEQ ID NO: 51.

74. An antibody or antigen binding fragment thereof that specifically binds to Pepsinogen C, comprising
(i) a VH that is at least 90% homologous to the VH as shown in SEQ ID NO: 50; and/or
(ii) a VL that is at least 90% homologous to the VL as shown in SEQ ID NO: 51.

75. An antibody or antigen binding fragment thereof that specifically binds to Pepsinogen C, comprising
(i) a VH that is at least 95% homologous to the VH as shown in SEQ ID NO: 50; and/or
(ii) a VL that is at least 95% homologous to the VL as shown in SEQ ID NO: 51.

76. An antibody or antigen binding fragment thereof that specifically binds to Pepsinogen C, comprising
(i) a VH that is at least 99% homologous to the VH as shown in SEQ ID NO: 50; and/or
(ii) a VL that is at least 99% homologous to the VL as shown in SEQ ID NO: 51.

77. An antibody or antigen binding fragment thereof that specifically binds to Pepsinogen C, comprising
(i) a VH comprising a sequence as shown in SEQ ID NO: 50; and/or
(ii) a VL comprising a sequence as shown in SEQ ID NO: 51.

78. An antibody or antigen binding fragment thereof that specifically binds to Pepsinogen C, comprising
(i) a VH consisting of the sequence as shown in SEQ ID NO: 50; and/or
(ii) a VL consisting of the sequence as shown in SEQ ID NO: 51.

79. An antibody or antigen binding fragment thereof that specifically binds to Pepsinogen C, comprising
(i) a VH comprising a CDR-H1 as shown in the amino acid sequence of SEQ ID NO: 3 or a variant thereof modified by one amino acid substitution; a CDR-H2 as shown in the amino acid sequence of SEQ ID NO: 4, or a variant thereof modified by one amino acid substitution; and a CDR-H3 as shown in the amino acid sequence of SEQ ID NO: 5, or a variant thereof modified by one amino acid substitution; and
(ii) a VL comprising a CDR-L1 as shown in the amino acid sequence of SEQ ID NO: 6 or a variant thereof modified by one amino acid substitution; a CDR-L2 as shown in the amino acid sequence of SEQ ID NO: 7, or a variant thereof modified by one amino acid substitution; and a CDR-L3 as shown in the amino acid sequence of SEQ ID NO: 52, or a variant thereof modified by one amino acid substitution.

80. The antibody or antigen binding fragment thereof according to item 79, comprising
(i) a VH comprising a CDR-H1 as shown in the amino acid sequence of SEQ ID NO: 3; a CDR-H2 as shown in the amino acid sequence of SEQ ID NO: 4; and a CDR-H3 as shown in the amino acid sequence of SEQ ID NO: 5; and
(ii) a VL comprising a CDR-L1 as shown in the amino acid sequence of SEQ ID NO: 6; a CDR-L2 as shown in the amino acid sequence of SEQ ID NO: 7; and a CDR-L3 as shown in the amino acid sequence of SEQ ID NO: 52.

81. The antibody or antigen binding fragment thereof according to item 79, wherein all the amino acid substitutions are conservative amino acid substitutions.

82. The antibody or antigen binding fragment thereof according to item 81, wherein each of said conservative amino acid substitutions is the substitution of an amino acid with another amino acid selected from its same group, wherein the groups of amino acids are
a) the nonpolar, hydrophobic amino acids consisting of Gly, Ala, Val, Leu, Ile, Phe, Try, Trp, and Met;
b) the polar, neutral amino acids consisting of Ser, Thr, Asn, and Gln;
c) the positively charged, basic amino acids consisting of Arg, Lys, and His, and
d) the negatively charged, amino acids consisting of Asp and Glu
wherein if Cys is to be conservatively substituted, it is substituted with Ser or Ala, and
wherein if Pro is to be conservatively substituted it is substituted with Ala.

83. The antibody or antigen binding fragment thereof according to item 82, wherein each of said conservative amino acid substitutions is a highly conservative amino acid substitution selected from
a) substitution of Ala with Val, Leu, Ile or Gly;
b) substitution of Arg with Lys;
c) substitution of Asn with Gln;
d) substitution of Asp with Glu;
e) substitution of Cys with Ser;
f) substitution of Gln with Asn;
g) substitution of Glu with Asp;
h) substitution of Gly with Ala;
i) substitution of His with Arg;
j) substitution of Ile with Leu, Val or Ala;
k) substitution of Leu with Ile, Val or Ala;
l) substitution of Lys with Arg;
m) substitution of Met with Leu, Ile or Val;
n) substitution of Phe with Try or Trp;
o) substitution of Pro with Ala;
p) substitution of Ser with Thr;
q) substitution of Thr with Ser;
r) substitution of Trp with Phe or Tyr;
s) substitution of Tyr with Phe or Trp; and
t) substitution of Val with Leu, Ile or Ala.

84. The antibody or antigen binding fragment thereof according to any one of items 79 to 83,
wherein the antibody comprises a VL consisting of framework regions (FW) and CDRs as represented in formula I:

(FW-L1)-(CDR-L1)-(FW-L2)-(CDR-L2)-(FW-L3)-(CDR-L3)-(FW-L4) (formula I)
and a VH consisting of FWs and CDRs as represented in formula II:

(FW-H1)-(CDR-H1)-(FW-H2)-(CDR-H2)-(FW-H3)-(CDR-H3)-(FW-H4) (formula II),
wherein the FWs comprise the following amino acid sequences or a variant thereof that is at least 85% homologous thereto:
FW-L1 the amino acid sequence of SEQ ID NO: 18;
FW-L2 the amino acid sequence of SEQ ID NO: 56;
FW-L3 the amino acid sequence of SEQ ID NO: 20;
FW-L4 the amino acid sequence of SEQ ID NO: 21;
FW-H1 the amino acid sequence of SEQ ID NO: 53;
FW-H2 the amino acid sequence of SEQ ID NO: 54;
FW-H3 the amino acid sequence of SEQ ID NO: 55;
FW-H4 the amino acid sequence of SEQ ID NO: 25.

85. The antibody or antigen binding fragment thereof according to any one of items 79 to 84,
wherein the antibody comprises a VL consisting of framework regions (FW) and CDRs as represented in formula I:

(FW-L1)-(CDR-L1)-(FW-L2)-(CDR-L2)-(FW-L3)-(CDR-L3)-(FW-L4) (formula I)
and a VH consisting of FWs and CDRs as represented in formula II:

(FW-H1)-(CDR-H1)-(FW-H2)-(CDR-H2)-(FW-H3)-(CDR-H3)-(FW-H4) (formula II),
wherein the FWs comprise the following amino acid sequences or a variant thereof modified by at most three amino acid substitutions:
FW-L1 the amino acid sequence of SEQ ID NO: 18;
FW-L2 the amino acid sequence of SEQ ID NO: 56;
FW-L3 the amino acid sequence of SEQ ID NO: 20;
FW-L4 the amino acid sequence of SEQ ID NO: 21;
FW-H1 the amino acid sequence of SEQ ID NO: 53;
FW-H2 the amino acid sequence of SEQ ID NO: 54;
FW-H3 the amino acid sequence of SEQ ID NO: 55;
FW-H4 the amino acid sequence of SEQ ID NO: 25.

86. The antibody or antigen binding fragment thereof according to item 85, wherein all the amino acid substitutions are conservative amino acid substitutions.

87. The antibody or antigen binding fragment thereof according to item 86, wherein each of said conservative amino acid substitutions is the substitution of an amino acid with another amino acid selected from its same group, wherein the groups of amino acids are
a) the nonpolar, hydrophobic amino acids consisting of Gly, Ala, Val, Leu, Ile, Phe, Try, Trp, and Met;
b) the polar, neutral amino acids consisting of Ser, Thr, Asn, and Gln;
c) the positively charged, basic amino acids consisting of Arg, Lys, and His, and
d) the negatively charged, amino acids consisting of Asp and Glu
wherein if Cys is to be conservatively substituted, it is substituted with Ser or Ala, and
wherein if Pro is to be conservatively substituted it is substituted with Ala.

88. The antibody or antigen binding fragment thereof according to item 87, wherein each of said conservative amino acid substitutions is a highly conservative amino acid substitution selected from
a) substitution of Ala with Val, Leu, Ile or Gly;
b) substitution of Arg with Lys;
c) substitution of Asn with Gln;
d) substitution of Asp with Glu;
e) substitution of Cys with Ser;
f) substitution of Gln with Asn;
g) substitution of Glu with Asp;
h) substitution of Gly with Ala;
i) substitution of His with Arg;
j) substitution of Ile with Leu, Val or Ala;
k) substitution of Leu with Ile, Val or Ala;
l) substitution of Lys with Arg;
m) substitution of Met with Leu, Ile or Val;
n) substitution of Phe with Try or Trp;
o) substitution of Pro with Ala;
p) substitution of Ser with Thr;
q) substitution of Thr with Ser;
r) substitution of Trp with Phe or Tyr;
s) substitution of Tyr with Phe or Trp; and
t) substitution of Val with Leu, Ile or Ala.

89. The antibody or antigen binding fragment thereof according to any one of items 72 to 88, comprising
(i) a VH comprising a sequence as shown in SEQ ID NO: 1; and/or
(ii) a VL comprising a sequence as shown in SEQ ID NO: 2.

90. The antibody or antigen binding fragment thereof according to any one of items 72 to 88,
comprising
(i) a VH comprising a sequence as shown in SEQ ID NO: 58; and/or
(ii) a VL comprising a sequence as shown in SEQ ID NO: 59.

91. The antibody or antigen binding fragment thereof according to any one of items 77 to 88,
comprising
(i) a VH comprising a sequence as shown in SEQ ID NO: 60; and/or
(ii) a VL comprising a sequence as shown in SEQ ID NO: 61.

92. The antibody or antigen binding fragment thereof according to any one of items 72 to 91, wherein the antibody or antigen binding fragment thereof binds to Pepsinogen C consisting of SEQ ID NO: 62 with a K_{D} of 0.1 nM or less, preferably wherein the K_{D} is measured at 37 °C.

93. The antibody or antigen binding fragment thereof according to any one of items 72 to 92, wherein the antibody or antigen binding fragment thereof binds to Pepsinogen C consisting of SEQ ID NO: 62 with t/2-diss at 37 °C of 85 minutes or longer.

94. The antibody or antigen binding fragment thereof according to any one of items 72 to 93, wherein the association rate kₐ for Pepsinogen C consisting of SEQ ID NO: 62 is at least 0.9×10⁶ M⁻¹s⁻¹.

95. An antibody or antigen binding fragment thereof that specifically binds to Pepsinogen C, comprising
(i) a heavy chain variable domain (VH) that is at least 80% homologous to the VH as shown in SEQ ID NO: 34; and
(ii) a light chain variable domain (VL) that is at least 80% homologous to the VL as shown in SEQ ID NO: 35.

96. An antibody or antigen binding fragment thereof that specifically binds to Pepsinogen C, comprising
(i) a VH that is at least 85% homologous to the VH as shown in SEQ ID NO: 34; and/or
(ii) a VL that is at least 85% homologous to the VL as shown in SEQ ID NO: 35.

97. An antibody or antigen binding fragment thereof that specifically binds to Pepsinogen C, comprising
(i) a VH that is at least 90% homologous to the VH as shown in SEQ ID NO: 34; and/or
(ii) a VL that is at least 90% homologous to the VL as shown in SEQ ID NO: 35.

98. An antibody or antigen binding fragment thereof that specifically binds to Pepsinogen C, comprising
(i) a VH that is at least 95% homologous to the VH as shown in SEQ ID NO: 34; and/or
(ii) a VL that is at least 95% homologous to the VL as shown in SEQ ID NO: 35.

99. An antibody or antigen binding fragment thereof that specifically binds to Pepsinogen C, comprising
(i) a VH that is at least 99% homologous to the VH as shown in SEQ ID NO: 34; and/or
(ii) a VL that is at least 99% homologous to the VL as shown in SEQ ID NO: 35.

100. An antibody or antigen binding fragment thereof that specifically binds to Pepsinogen C, comprising
(i) a VH comprising a sequence as shown in SEQ ID NO: 34; and/or
(ii) a VL comprising a sequence as shown in SEQ ID NO: 35.

101. An antibody or antigen binding fragment thereof that specifically binds to Pepsinogen C, comprising
(i) a VH consisting of the sequence as shown in SEQ ID NO: 34; and/or
(ii) a VL consisting of the sequence as shown in SEQ ID NO: 35.

102. An antibody or antigen binding fragment thereof that specifically binds to Pepsinogen C, comprising
(i) a VH comprising a CDR-H1 as shown in the amino acid sequence of SEQ ID NO: 36 or a variant thereof modified by one amino acid substitution; a CDR-H2 as shown in the amino acid sequence of SEQ ID NO: 37, or a variant thereof modified by one amino acid substitution; and a CDR-H3 as shown in the amino acid sequence of SEQ ID NO: 38, or a variant thereof modified by one amino acid substitution; and
(ii) a VL comprising a CDR-L1 as shown in the amino acid sequence of SEQ ID NO: 39 or a variant thereof modified by one amino acid substitution; a CDR-L2 as shown in the amino acid sequence of SEQ ID NO: 40, or a variant thereof modified by one amino acid substitution; and a CDR-L3 as shown in the amino acid sequence of SEQ ID NO: 41, or a variant thereof modified by one amino acid substitution.

103. The antibody or antigen binding fragment thereof according to item 102, wherein all the amino acid substitutions are conservative amino acid substitutions.

104. The antibody or antigen binding fragment thereof according to item 103, wherein each of said conservative amino acid substitutions is the substitution of an amino acid with another amino acid selected from its same group, wherein the groups of amino acids are
a) the nonpolar, hydrophobic amino acids consisting of Gly, Ala, Val, Leu, Ile, Phe, Try, Trp, and Met;
b) the polar, neutral amino acids consisting of Ser, Thr, Asn, and Gln;
c) the positively charged, basic amino acids consisting of Arg, Lys, and His, and
d) the negatively charged, amino acids consisting of Asp and Glu
wherein if Cys is to be conservatively substituted, it is substituted with Ser or Ala, and
wherein if Pro is to be conservatively substituted it is substituted with Ala.

105. The antibody or antigen binding fragment thereof according to item 103 or 104, wherein each of said conservative amino acid substitutions is a highly conservative amino acid substitution selected from
a) substitution of Ala with Val, Leu, Ile or Gly;
b) substitution of Arg with Lys;
c) substitution of Asn with Gln;
d) substitution of Asp with Glu;
e) substitution of Cys with Ser;
f) substitution of Gln with Asn;
g) substitution of Glu with Asp;
h) substitution of Gly with Ala;
i) substitution of His with Arg;
j) substitution of Ile with Leu, Val or Ala;
k) substitution of Leu with Ile, Val or Ala;
l) substitution of Lys with Arg;
m) substitution of Met with Leu, Ile or Val;
n) substitution of Phe with Try or Trp;
o) substitution of Pro with Ala;
p) substitution of Ser with Thr;
q) substitution of Thr with Ser;
r) substitution of Trp with Phe or Tyr;
s) substitution of Tyr with Phe or Trp; and
t) substitution of Val with Leu, Ile or Ala.

106. The antibody or antigen binding fragment thereof according to item 102, wherein the antibody, or antigen binding fragments thereof, comprises
(i) a VH comprising a CDR-H1 as shown in the amino acid sequence of SEQ ID NO: 36; a CDR-H2 as shown in the amino acid sequence of SEQ ID NO: 37; and a CDR-H3 as shown in the amino acid sequence of SEQ ID NO: 38; and
(ii) a VL comprising a CDR-L1 as shown in the amino acid sequence of SEQ ID NO: 39; a CDR-L2 as shown in the amino acid sequence of SEQ ID NO: 40; and a CDR-L3 as shown in the amino acid sequence of SEQ ID NO: 41.

107. The antibody or antigen binding fragment thereof according to any one of items 102 to 106,
wherein the antibody comprises a VL consisting of framework regions (FW) and CDRs as represented in formula I:

(FW-L1)-(CDR-L1)-(FW-L2)-(CDR-L2)-(FW-L3)-(CDR-L3)-(FW-L4) (formula I)
and a VH consisting of FWs and CDRs as represented in formula II:

(FW-H1)-(CDR-H1)-(FW-H2)-(CDR-H2)-(FW-H3)-(CDR-H3)-(FW-H4) (formula II),
wherein the FWs comprise the following amino acid sequences or a variant thereof that is at least 85% homologous thereto:
FW-L1 the amino acid sequence of SEQ ID NO: 42;
FW-L2 the amino acid sequence of SEQ ID NO: 43;
FW-L3 the amino acid sequence of SEQ ID NO: 44;
FW-L4 the amino acid sequence of SEQ ID NO: 45;
FW-H1 the amino acid sequence of SEQ ID NO: 46;
FW-H2 the amino acid sequence of SEQ ID NO: 47;
FW-H3 the amino acid sequence of SEQ ID NO: 48;
FW-H4 the amino acid sequence of SEQ ID NO: 49.

108. The antibody or antigen binding fragment thereof according to any one of items 102 to 107,
wherein the antibody comprises a VL consisting of framework regions (FW) and CDRs as represented in formula I:

(FW-L1)-(CDR-L1)-(FW-L2)-(CDR-L2)-(FW-L3)-(CDR-L3)-(FW-L4) (formula I)
and a VH consisting of FWs and CDRs as represented in formula II:

(FW-H1)-(CDR-H1)-(FW-H2)-(CDR-H2)-(FW-H3)-(CDR-H3)-(FW-H4) (formula II),
wherein the FWs comprise the following amino acid sequences or a variant thereof modified by at most three amino acid substitutions:
FW-L1 the amino acid sequence of SEQ ID NO: 42;
FW-L2 the amino acid sequence of SEQ ID NO: 43;
FW-L3 the amino acid sequence of SEQ ID NO: 44;
FW-L4 the amino acid sequence of SEQ ID NO: 45;
FW-H1 the amino acid sequence of SEQ ID NO: 46;
FW-H2 the amino acid sequence of SEQ ID NO: 47;
FW-H3 the amino acid sequence of SEQ ID NO: 48;
FW-H4 the amino acid sequence of SEQ ID NO: 49.

109. The antibody, or antigen binding fragment thereof, according to item 108, wherein all the amino acid substitutions are conservative amino acid substitutions.

110. The antibody, or antigen binding fragment thereof, according to item 109, wherein each of said conservative amino acid substitutions is the substitution of an amino acid with another amino acid selected from its same group, wherein the groups of amino acids are
a) the nonpolar, hydrophobic amino acids consisting of Gly, Ala, Val, Leu, Ile, Phe, Try, Trp, and Met;
b) the polar, neutral amino acids consisting of Ser, Thr, Asn, and Gln;
c) the positively charged, basic amino acids consisting of Arg, Lys, and His, and
d) the negatively charged, amino acids consisting of Asp and Glu
wherein if Cys is to be conservatively substituted, it is substituted with Ser or Ala, and
wherein if Pro is to be conservatively substituted it is substituted with Ala.

111. The antibody or antigen binding fragment thereof according to item 109 or 110, wherein each of said conservative amino acid substitutions is a highly conservative amino acid substitution selected from
a) substitution of Ala with Val, Leu, Ile or Gly;
b) substitution of Arg with Lys;
c) substitution of Asn with Gln;
d) substitution of Asp with Glu;
e) substitution of Cys with Ser;
f) substitution of Gln with Asn;
g) substitution of Glu with Asp;
h) substitution of Gly with Ala;
i) substitution of His with Arg;
j) substitution of Ile with Leu, Val or Ala;
k) substitution of Leu with Ile, Val or Ala;
l) substitution of Lys with Arg;
m) substitution of Met with Leu, Ile or Val;
n) substitution of Phe with Try or Trp;
o) substitution of Pro with Ala;
p) substitution of Ser with Thr;
q) substitution of Thr with Ser;
r) substitution of Trp with Phe or Tyr;
s) substitution of Tyr with Phe or Trp; and
t) substitution of Val with Leu, Ile or Ala.

112. The antibody or antigen binding fragment thereof according to any one of items 95 to 111, wherein the antibody or antigen binding fragment thereof binds to Pepsinogen C consisting of SEQ ID NO: 62 with a K_{D} of 0.6 nM or less, preferably wherein the K_{D} is measured at 37 °C.

113. The antibody or antigen binding fragment thereof according to any one of items 95 to 112, wherein the antibody or antigen binding fragment thereof binds to Pepsinogen C consisting of SEQ ID NO: 62 with t/2-diss at 37 °C of 75 minutes or longer.

114. The antibody or antigen binding fragment thereof according to any one of items 95 to 113, wherein the association rate kₐ for Pepsinogen C consisting of SEQ ID NO: 62 is at least 2.6×10⁵ M⁻¹s⁻¹.

115. An antibody or antigen binding fragment thereof that specifically binds to Pepsinogen C, comprising
(i) a heavy chain variable domain (VH) that is at least 80% homologous to the VH as shown in SEQ ID NO: 9; and
(ii) a light chain variable domain (VL) that is at least 80% homologous to the VL as shown in SEQ ID NO: 10.

116. An antibody or antigen binding fragment thereof that specifically binds to Pepsinogen C, comprising
(i) a VH that is at least 85% homologous to the VH as shown in SEQ ID NO: 9; and/or
(ii) a VL that is at least 85% homologous to the VL as shown in SEQ ID NO: 10.

117. An antibody or antigen binding fragment thereof that specifically binds to Pepsinogen C, comprising
(i) a VH that is at least 90% homologous to the VH as shown in SEQ ID NO: 9; and/or
(ii) a VL that is at least 90% homologous to the VL as shown in SEQ ID NO: 10.

118. An antibody or antigen binding fragment thereof that specifically binds to Pepsinogen C, comprising
(i) a VH that is at least 95% homologous to the VH as shown in SEQ ID NO: 9; and/or
(ii) a VL that is at least 95% homologous to the VL as shown in SEQ ID NO: 10.

119. An antibody or antigen binding fragment thereof that specifically binds to Pepsinogen C, comprising (i) a VH comprising a sequence as shown in SEQ ID NO: 9; and/or
(ii) a VL comprising a sequence as shown in SEQ ID NO: 10.

120. An antibody or antigen binding fragment thereof that specifically binds to Pepsinogen C, comprising
(i) a VH consisting of the sequence as shown in SEQ ID NO: 9; and/or
(ii) a VL consisting of the sequence as shown in SEQ ID NO: 10.

121. An antibody or antigen binding fragment thereof that specifically binds to Pepsinogen C, comprising
(i) a VH comprising a CDR-H1 as shown in the amino acid sequence of SEQ ID NO: 11 or a variant thereof modified by one amino acid substitution; a CDR-H2 as shown in the amino acid sequence of SEQ ID NO: 12, or a variant thereof modified by one amino acid substitution; and a CDR-H3 as shown in the amino acid sequence of SEQ ID NO: 13, or a variant thereof modified by one amino acid substitution; and
(ii) a VL comprising a CDR-L1 as shown in the amino acid sequence of SEQ ID NO: 14 or a variant thereof modified by one amino acid substitution; a CDR-L2 as shown in the amino acid sequence of SEQ ID NO: 15, or a variant thereof modified by one amino acid substitution; and a CDR-L3 as shown in the amino acid sequence of SEQ ID NO: 16, or a variant thereof modified by one amino acid substitution.

122. The antibody or antigen binding fragment thereof according to item 121, wherein the antibody, or antigen binding fragments thereof, comprises
(i) a VH comprising a CDR-H1 as shown in the amino acid sequence of SEQ ID NO: 11; a CDR-H2 as shown in the amino acid sequence of SEQ ID NO: 12; and a CDR-H3 as shown in the amino acid sequence of SEQ ID NO: 13; and
(ii) a VL comprising a CDR-L1 as shown in the amino acid sequence of SEQ ID NO: 14; a CDR-L2 as shown in the amino acid sequence of SEQ ID NO: 15; and a CDR-L3 as shown in the amino acid sequence of SEQ ID NO: 16.

123. The antibody or antigen binding fragment thereof according to item 121 or 122,
wherein the antibody comprises a VL consisting of framework regions (FW) and CDRs as represented in formula I:

(FW-L1)-(CDR-L1)-(FW-L2)-(CDR-L2)-(FW-L3)-(CDR-L3)-(FW-L4) (formula I)
and a VH consisting of FWs and CDRs as represented in formula II:

(FW-H1)-(CDR-H1)-(FW-H2)-(CDR-H2)-(FW-H3)-(CDR-H3)-(FW-H4) (formula II),
wherein the FWs comprise the following amino acid sequences or a variant thereof that is at least 85% homologous thereto:
FW-L1 the amino acid sequence of SEQ ID NO: 26;
FW-L2 the amino acid sequence of SEQ ID NO: 27;
FW-L3 the amino acid sequence of SEQ ID NO: 28;
FW-L4 the amino acid sequence of SEQ ID NO: 29;
FW-H1 the amino acid sequence of SEQ ID NO: 30;
FW-H2 the amino acid sequence of SEQ ID NO: 31;
FW-H3 the amino acid sequence of SEQ ID NO: 32;
FW-H4 the amino acid sequence of SEQ ID NO: 33.

124. The antibody or antigen binding fragment thereof according to any one of items 121 to 123,
wherein the antibody comprises a VL consisting of framework regions (FW) and CDRs as represented in formula I:

(FW-L1)-(CDR-L1)-(FW-L2)-(CDR-L2)-(FW-L3)-(CDR-L3)-(FW-L4) (formula I)
and a VH consisting of FWs and CDRs as represented in formula II:

(FW-H1)-(CDR-H1)-(FW-H2)-(CDR-H2)-(FW-H3)-(CDR-H3)-(FW-H4) (formula II),
wherein the FWs comprise the following amino acid sequences or a variant thereof modified by at most three amino acid substitutions:
FW-L1 the amino acid sequence of SEQ ID NO: 26;
FW-L2 the amino acid sequence of SEQ ID NO: 27;
FW-L3 the amino acid sequence of SEQ ID NO: 28;
FW-L4 the amino acid sequence of SEQ ID NO: 29;
FW-H1 the amino acid sequence of SEQ ID NO: 30;
FW-H2 the amino acid sequence of SEQ ID NO: 31;
FW-H3 the amino acid sequence of SEQ ID NO: 32;
FW-H4 the amino acid sequence of SEQ ID NO: 33.

125. The antibody or antigen binding fragment thereof according to item 121 or 124, wherein all the amino acid substitutions are conservative amino acid substitutions.

126. The antibody or antigen binding fragment thereof according to item 125, wherein each of said conservative amino acid substitutions is the substitution of an amino acid with another amino acid selected from its same group, wherein the groups of amino acids are
a) the nonpolar, hydrophobic amino acids consisting of Gly, Ala, Val, Leu, Ile, Phe, Try, Trp, and Met;
b) the polar, neutral amino acids consisting of Ser, Thr, Asn, and Gln;
c) the positively charged, basic amino acids consisting of Arg, Lys, and His, and
d) the negatively charged, amino acids consisting of Asp and Glu
wherein if Cys is to be conservatively substituted, it is substituted with Ser or Ala, and
wherein if Pro is to be conservatively substituted it is substituted with Ala.

127. The antibody or antigen binding fragment thereof according to any one of items 125 to 126, wherein each of said conservative amino acid substitutions is a highly conservative amino acid substitution selected from
a) substitution of Ala with Val, Leu, Ile or Gly;
b) substitution of Arg with Lys;
c) substitution of Asn with Gln;
d) substitution of Asp with Glu;
e) substitution of Cys with Ser;
f) substitution of Gln with Asn;
g) substitution of Glu with Asp;
h) substitution of Gly with Ala;
i) substitution of His with Arg;
j) substitution of Ile with Leu, Val or Ala;
k) substitution of Leu with Ile, Val or Ala;
l) substitution of Lys with Arg;
m) substitution of Met with Leu, Ile or Val;
n) substitution of Phe with Try or Trp;
o) substitution of Pro with Ala;
p) substitution of Ser with Thr;
q) substitution of Thr with Ser;
r) substitution of Trp with Phe or Tyr;
s) substitution of Tyr with Phe or Trp; and
t) substitution of Val with Leu, Ile or Ala.

128. The antibody or antigen binding fragment thereof according to any one of items 115 to 127, wherein the antibody or antigen binding fragment thereof binds to Pepsinogen C consisting of SEQ ID NO: 62 with a K_{D} of 0.3 nM or less, preferably wherein the K_{D} is measured at 37 °C.

129. The antibody or antigen binding fragment thereof according to any one of items 115 to 128, wherein the antibody or antigen binding fragment thereof binds to Pepsinogen C consisting of SEQ ID NO: 62 with t/2-diss at 37 °C of 30 minutes or longer.

130. The antibody or antigen binding fragment thereof according to any one of items 115 to 129, wherein the association rate kₐ for Pepsinogen C consisting of SEQ ID NO: 62 is at least 1.4×10⁶ M⁻¹s⁻¹.

131. The antibody or antigen binding fragment thereof according to any one of items 72 to 130, wherein the antibody is a monoclonal antibody.

132. The antibody or antigen binding fragment thereof according to any one of items 72 to 131, wherein the Pepsinogen C comprises SEQ ID NO: 17, preferably wherein the Pepsinogen C consists of SEQ ID NO: 17.

133. The antibody or antigen binding fragment thereof according to any one of items 724 to 91, 95 to 111, 115 to 127, and 131 to 132, wherein the antibody or antigen binding fragment thereof binds to Pepsinogen C consisting of SEQ ID NO: 62 with a KD of 2 nM or less, or 1.5 nM or less, or 1 nM or less, or 0.6 nM or less, preferably wherein the K_{D} is measured at 37 °C.

134. The antibody or antigen binding fragment thereof according to any one of items 72 to 91, 95 to 111, 115 to 127, and 131 to 133, wherein the antibody or antigen binding fragment thereof binds to Pepsinogen C consisting of SEQ ID NO: 62 with t/2-diss at 37 °C of 20 minutes or longer, preferably 30 minutes or longer for binding to Pepsinogen C consisting of SEQ ID NO: 62.

135. The antibody or antigen binding fragment thereof according to any one of items 72 to 91, 95 to 111, 115 to 127, and 131 to 134, wherein the association rate kₐ for Pepsinogen C comprising SEQ ID NO: 62 is at least 10⁵ M⁻¹s⁻¹, preferable at least 2.5×10⁵ M⁻¹s⁻¹.

136. The antibody or antigen binding fragment thereof according to any one of items 133 to 135, wherein said K_{D} and/or said kₐ is determined by surface plasmon resonance spectroscopy.

137. The antibody or antigen binding fragment thereof according to item 136, wherein said surface plasmon resonance spectroscopy comprises attaching or capturing the antibody, or antigen binding fragment thereof, on a CM5 sensor chip and injecting Pepsinogen C comprising SEQ ID NO: 62 as analyte, wherein said determination is conducted at a temperature of 37 °C using HBS-ET buffer (10 mM HEPES, 150 mM NaCl, 3 mM EDTA, 0.05% (w/v) Tween 20®).

138. The antibody or antigen binding fragment thereof according to item 136 or 137, wherein the wherein the antibody does not specifically bind to Pepsinogen A.

139. The antibody or antigen binding fragment thereof according to any one of items 136 to 138, wherein the surface plasmon resonance spectroscopy is performed with a Biacore 8 k or B4000 instrument.

140. The antibody or antigen binding fragment thereof according to any one of items 136 to 139, wherein the determination of said KD and/or said ka comprises fitting the surface plasmon resonance data using a Langmuir fitting model.

141. A polynucleotide or a set of polynucleotides encoding
(i) the heavy chain variable domain of the antibody or antigen binding fragment thereof according to any one of items 72 to 140, and/or
(ii) the light chain variable domain of the antibody or antigen binding fragment according to any one of items 72 to 140.

142. A vector comprising the polynucleotide or the set of polynucleotides according to item 141.

143. A host cell comprising the polynucleotide or the set of polynucleotides according to item 141, or the vector according to item 142 and expressing the antibody or antigen binding fragment thereof according to any one of items 72 to 140.

144. A host cell according to item 143, wherein the host cell is a prokaryotic cell or a eukaryotic cell.

145. A host cell according to item 144, wherein the host cell is a eukaryotic cell, wherein said host cell is a HEK293 cell or a CHO cell.

146. A method of producing the antibody or antigen binding fragment thereof according to any one of items 72 to 140 comprising culturing the host cell according to any one of items 143 to 145 and isolating said antibody or antigen binding fragment thereof.

147. An antibody or an antigen binding fragment thereof according to any one of items 72 to 140 obtainable by the method of item 146.

148. A composition comprising the antibody or antigen binding fragment thereof according to any one of items 72-140 or 147, the polynucleotide or set of polynucleotides according to item 141, the vector according to item 142, or the host cell according to any one of items 143 to 145.

149. A composition comprising
a) the antibody or antigen binding fragment thereof according to any one of items 72 to 114 or according to any one of items 131-140 or 147 as dependent from any one of items 72 to 114;
and the antibody or antigen binding fragment thereof according to any one of items 115 to 130 or according to any one of items 131-140 or 147 as dependent from any one of items 115 to 130;
b) the polynucleotide or set of polynucleotides of item 141 as dependent from any one of items 72 to 114 or according to any one of items 131-140 or 147 as dependent from any one of items 72 to 114;
and the polynucleotide or set of polynucleotides of item 141 as dependent from any one of items 115 to 130 or according to any one of items 131-140 or 147 as dependent from any one of items 115 to 130;
c) the vector of item 142 as dependent from any one of items 72 to 114 or according to any one of items 131-140 or 147 as dependent from any one of items 72 to 114;
and the vector of item 142 as dependent from any one of items 115 to 130 or according to any one of items 131-140 or 147 as dependent from any one of items 115 to 130; or
d) the host cell according to any one of items 143 to 145 as dependent from any one of items 72 to 114 or according to any one of items 131-140 or 147 as dependent from any one of items 72 to 114;
and the host cell according to any one of items 143 to 145 as dependent from any one of items 115 to 130 or according to any one of items 131-140 or 147 as dependent from any one of items 115 to 130.

150. A composition comprising the antibody or antigen binding fragment thereof according to any one of items 72-140 or 147, wherein the composition is a diagnostic composition.

151. A composition comprising
a) the antibody or antigen binding fragment thereof according to any one of items 72 to 114 or according to any one of items 131-140 or 147 as dependent from any one of items 72 to 114; and
b) the antibody or antigen binding fragment thereof according to any one of items 115 to 130 or according to any one of items 131-140 or 147 as dependent from any one of items 115 to 130;
wherein the composition is a diagnostic composition.

152. Use of the antibody or antigen binding fragment thereof according to any one of items 72-140 or 147 or the composition according to item 150 or 151 for an in vitro immunoassay, in particular an in vitro immunoassay for detecting Pepsinogen C in a sample.

153. Use of the antibody or antigen binding fragment thereof according to item 152, wherein the immunoassay is a heterogeneous immunoassay.

154. Use of the antibody or antigen binding fragment thereof according to item 142 or 153, wherein the sample for said immunoassay is a sample prepared from blood, plasma, or serum.

155. An in vitro method for detecting Pepsinogen C in a sample, comprising
(a) contacting a sample comprising Pepsinogen C with the antibody, or antigen binding fragment thereof, according to any one of items 72-140 or 147, thereby forming a complex comprising Pepsinogen C and said antibody, or antigen binding fragment thereof, and
(b) detecting the complex formed in step (a), thereby detecting Pepsinogen C in said sample.

156. The method according to item 155, wherein the method is a serum immunoassay, wherein the sample is a body fluid, preferably whole blood, serum or plasma.

157. The method according to item 155 or 156, wherein the antibody, or antigen binding fragment thereof, has a detection label attached thereto.

158. The method according to item 157, wherein the detection label is selected from an enzyme, a label emitting light, preferable fluorescence, luminescence, chemiluminescence, electrochemiluminescence, or radioactivity.

159. An in vitro method for detecting Pepsinogen C in a sample, comprising the following steps:
1) contacting a sample comprising Pepsinogen C with a first antibody, or antigen binding fragment thereof, and a second antibody, or antigen binding fragment thereof, wherein
(a) the first antibody is
(i) the antibody or antigen binding fragment thereof according to any one of items 72 to 114 or according to any one of items 131-140 or 147 as dependent from any one of items 72 to 114; or
(ii) is the antibody or antigen binding fragment thereof according to any one of items 115 to 130 or according to any one of items 131-140 or 147 as dependent from any one of items 115 to 130;
(b) the second antibody specifically binds to Pepsinogen C and does not compete with the first antibody for specifically binding to Pepsinogen C; and

2) incubating the resulting mixture of 1) to form a complex comprising Pepsinogen C and said first antibody or antigen binding fragment thereof and said second antibody or antigen binding fragment thereof; and
3) detecting Pepsinogen C in the sample by detecting the formation of the complex as defined in 2).

160. The method according to item 159, wherein (c) one of the first and the second antibody comprises a detection label.

161. The method according to item 159 or 160, wherein the first antibody or antigen binding fragment thereof or the second antibody or antigen binding fragment thereof not comprising the detection label, comprises a capture label.

162. The method according to item 161, wherein the method further comprises adding to the mixture of (1) particles capable of binding to the capture label and 2.1) separating the particles from the mixture before detecting a signal provided by the detection label.

163. The method according to any one of items 159 to 162, wherein
when the first antibody or antigen binding fragment thereof is selected from (i) than the second antibody is selected from (ii); or
when the first antibody or antigen binding fragment thereof is selected from (ii) than the second antibody is selected from (i).

164. The method according to any one of items 159 to 163, wherein the method is a serum immunoassay, wherein the sample is a body fluid, preferably whole blood, serum or plasma.

165. The method according to any one of items 159 to 164, wherein the detection label is selected from an enzyme, a label emitting light, preferable fluorescence, luminescence, chemiluminescence, electrochemiluminescence, or radioactivity.

166. The method according to any one of items 159 to 165, wherein the Limit of Quantification (LoQ) is 0.353 ng/mL at 20% CV.

167. The method according to any one of items 159 to 166, wherein the detection of Pepsinogen C in the sample is achieved in 18 minutes or less.

168. A kit comprising the antibody, or the antigen binding fragment thereof, according to any one of items 72-140 or 147.

169. A kit comprising
1) the composition according to item 151, or
2) a first antibody, or antigen binding fragment thereof, and a second antibody, or antigen binding fragment thereof, wherein one of the first or second antibody, or antigen binding fragment thereof, is
a) the antibody or antigen binding fragment thereof according to any one of items 72 to 114 or according to any one of items 131-140 or 147 as dependent from any one of items 72 to 114;
and the other of the first or second antibody, or antigen binding fragment thereof is
b) the antibody or antigen binding fragment thereof according to any one of items 115 to 130 or according to any one of items 131-140 or 147 as dependent from any one of items 115 to 130.

170. The kit according to item 169, wherein one of the first and the second antibody, or antigen binding fragment thereof, comprises a detection label.

171. The kit according to item 170, wherein the first antibody, or antigen binding fragment thereof, or the second antibody, or antigen binding fragment thereof, not comprising the detection label, comprises a capture label.

172. The kit according to any one of items 168 to 171, wherein the kit is for detecting and/or quantifying Pepsinogen C.

173. The kit according to any one of items 168 to 172 that is an immunoassay kit.

174. A kit comprising a first antibody or antigen binding fragment thereof and a second antibody or antigen binding fragment thereof,
a) wherein the first antibody or antigen binding fragment thereof is
an antibody or antigen binding fragment thereof that specifically binds to Pepsinogen C and competes for binding to Pepsinogen C with the antibody consisting of
(i) a heavy chain variable domain (VH) as shown in SEQ ID NO: 1; and
(ii) a light chain variable domain (VL) as shown in SEQ ID NO: 2; and
(b) wherein the second antibody or antigen binding fragment thereof is
an antibody or antigen binding fragment thereof that specifically binds to Pepsinogen C and competes for binding to Pepsinogen C, consisting of
(iii) a heavy chain variable domain (VH) as shown in SEQ ID NO: 9; and
(iv) a light chain variable domain (VL) as shown in SEQ ID NO: 10; and
wherein the second antibody or antigen binding fragments thereof does not compete for binding to Pepsinogen C with the first antibody or antigen binding fragment thereof.

175. The kit of item 174, wherein the first antibody or antigen binding fragment comprises
(a) a VH comprising a CDR-H1 as shown in the amino acid sequence of SEQ ID NO: 3 or a variant thereof modified by one amino acid substitution; a CDR-H2 as shown in the amino acid sequence of SEQ ID NO: 4, or a variant thereof modified by one amino acid substitution; and a CDR-H3 as shown in the amino acid sequence of SEQ ID NO: 5, or a variant thereof modified by one amino acid substitution; and
a VL comprising a CDR-L1 as shown in the amino acid sequence of SEQ ID NO: 6 or a variant thereof modified by one amino acid substitution; a CDR-L2 as shown in the amino acid sequence of SEQ ID NO: 7, or a variant thereof modified by one amino acid substitution; and a CDR-L3 as shown in the amino acid sequence of SEQ ID NO: 52, or a variant thereof modified by one amino acid substitution; or
(b) a VH comprising a CDR-H1 as shown in the amino acid sequence of SEQ ID NO: 36 or a variant thereof modified by one amino acid substitution; a CDR-H2 as shown in the amino acid sequence of SEQ ID NO: 37, or a variant thereof modified by one amino acid substitution; and a CDR-H3 as shown in the amino acid sequence of SEQ ID NO: 38, or a variant thereof modified by one amino acid substitution; and
a VL comprising a CDR-L1 as shown in the amino acid sequence of SEQ ID NO: 39 or a variant thereof modified by one amino acid substitution; a CDR-L2 as shown in the amino acid sequence of SEQ ID NO: 40, or a variant thereof modified by one amino acid substitution; and a CDR-L3 as shown in the amino acid sequence of SEQ ID NO: 41, or a variant thereof modified by one amino acid substitution; and/or
wherein the second antibody, or antigen binding fragment thereof, comprises
(c) a VH comprising a CDR-H1 as shown in the amino acid sequence of SEQ ID NO: 11 or a variant thereof modified by one amino acid substitution; a CDR-H2 as shown in the amino acid sequence of SEQ ID NO: 12, or a variant thereof modified by one amino acid substitution; and a CDR-H3 as shown in the amino acid sequence of SEQ ID NO: 13, or a variant thereof modified by one amino acid substitution; and
VL comprising a CDR-L1 as shown in the amino acid sequence of SEQ ID NO: 14 or a variant thereof modified by one amino acid substitution; a CDR-L2 as shown in the amino acid sequence of SEQ ID NO: 15, or a variant thereof modified by one amino acid substitution; and a CDR-L3 as shown in the amino acid sequence of SEQ ID NO: 16, or a variant thereof modified by one amino acid substitution.

176. An antibody or antigen binding fragment thereof that specifically binds to Pepsinogen C and competes for binding to Pepsinogen C with the antibody consisting of
(i) a heavy chain variable domain (VH) as shown in SEQ ID NO: 1; and
(ii) a light chain variable domain (VL) as shown in SEQ ID NO: 2.

177. The antibody or antigen binding fragment thereof according to item 176, comprising
(a) a VH comprising a CDR-H1 as shown in the amino acid sequence of SEQ ID NO: 3 or a variant thereof modified by one amino acid substitution; a CDR-H2 as shown in the amino acid sequence of SEQ ID NO: 4, or a variant thereof modified by one amino acid substitution; and a CDR-H3 as shown in the amino acid sequence of SEQ ID NO: 5, or a variant thereof modified by one amino acid substitution; and
a VL comprising a CDR-L1 as shown in the amino acid sequence of SEQ ID NO: 6 or a variant thereof modified by one amino acid substitution; a CDR-L2 as shown in the amino acid sequence of SEQ ID NO: 7, or a variant thereof modified by one amino acid substitution; and a CDR-L3 as shown in the amino acid sequence of SEQ ID NO: 52, or a variant thereof modified by one amino acid substitution; or
(b) a VH comprising a CDR-H1 as shown in the amino acid sequence of SEQ ID NO: 36 or a variant thereof modified by one amino acid substitution; a CDR-H2 as shown in the amino acid sequence of SEQ ID NO: 37, or a variant thereof modified by one amino acid substitution; and a CDR-H3 as shown in the amino acid sequence of SEQ ID NO: 38, or a variant thereof modified by one amino acid substitution; and
a VL comprising a CDR-L1 as shown in the amino acid sequence of SEQ ID NO: 39 or a variant thereof modified by one amino acid substitution; a CDR-L2 as shown in the amino acid sequence of SEQ ID NO: 40, or a variant thereof modified by one amino acid substitution; and a CDR-L3 as shown in the amino acid sequence of SEQ ID NO: 41, or a variant thereof modified by one amino acid substitution.

178. An antibody or antigen binding fragment thereof that specifically binds to Pepsinogen C and competes for binding to Pepsinogen C with the antibody consisting of
(i) a heavy chain variable domain (VH) as shown in SEQ ID NO: 9; and
(ii) a light chain variable domain (VL) as shown in SEQ ID NO: 10.

179. The antibody or antigen binding fragment thereof of item 178, comprising
a VH comprising a CDR-H1 as shown in the amino acid sequence of SEQ ID NO: 11 or a variant thereof modified by one amino acid substitution; a CDR-H2 as shown in the amino acid sequence of SEQ ID NO: 12, or a variant thereof modified by one amino acid substitution; and a CDR-H3 as shown in the amino acid sequence of SEQ ID NO: 13, or a variant thereof modified by one amino acid substitution; and
a VL comprising a CDR-L1 as shown in the amino acid sequence of SEQ ID NO: 14 or a variant thereof modified by one amino acid substitution; a CDR-L2 as shown in the amino acid sequence of SEQ ID NO: 15, or a variant thereof modified by one amino acid substitution; and a CDR-L3 as shown in the amino acid sequence of SEQ ID NO: 16, or a variant thereof modified by one amino acid substitution.

180. A polynucleotide or a set of polynucleotides encoding
(i) the heavy chain variable domain of the antibody or antigen binding fragment thereof according to any one of items 176 to 179, and/or
(ii) the light chain variable domain of the antibody or antigen binding fragment according to any one of items 176 to 179.

181. A vector comprising the polynucleotide or the set of polynucleotides according to item 180.

182. A host cell comprising the polynucleotide or the set of polynucleotides according to item 180, or the vector according to item 181 and expressing the antibody or antigen binding fragment thereof according to any one of items 176 to 179.

183. A method of producing the antibody or antigen binding fragment thereof according to any one of items 176 to 179 comprising culturing the host cell according to item 182 and isolating said antibody or antigen binding fragment thereof

184. An antibody or an antigen binding fragment thereof according to any one of items 176 to 179 obtainable by the method of item 183.

185. A composition comprising the antibody or antigen binding fragment thereof according to any one of items 176-179 or 184, the polynucleotide or set of polynucleotides according to item 180, the vector according to item 181, or the host cell according to item 182.

186. A composition comprising
a) the antibody or antigen binding fragment thereof according to item 171 or 172 or according to item 179 as dependent from item 176 or 177; and the antibody or antigen binding fragment thereof according to item 178 or 179 or according to item 184 as dependent from item 178 or 179.

187. Use of the antibody or antigen binding fragment thereof according to any one of claims 176 to 179 or 184, the composition according to claim 185 or 186 or the kit according to claim 174 or 175 for an in vitro immunoassay, in particular an in vitro immunoassay for detecting Pepsinogen C in a sample.

188. An in vitro method for detecting Pepsinogen C in a sample, comprising
(a) contacting a sample comprising Pepsinogen C with the antibody or antigen binding fragment thereof according to any one of items 176-179 or 184, thereby forming a complex comprising Pepsinogen C and said antibody or antigen binding fragment thereof, and
(b) detecting the complex formed in step (a), thereby detecting Pepsinogen C in said sample.

189. An in vitro method for detecting Pepsinogen C in a sample, comprising the following steps:
1) contacting a sample comprising Pepsinogen C with a first antibody or antigen binding fragment thereof and a second antibody or antigen binding fragment thereof, wherein
(a) the first antibody is
(i) the antibody or antigen binding fragment thereof according to item 176 or 177 or according to item 184 as dependent from item 176 or 177, or
(ii) the antibody or antigen binding fragment thereof according to item 178 or 179 or according to item 184 as dependent from item 178 or 179; and
(b) the second antibody specifically binds to Pepsinogen C and does not compete with the first antibody for specifically binding to Pepsinogen C;
2) incubating the resulting mixture of 1) to form a complex comprising Pepsinogen C and said first antibody or antigen binding fragment thereof and said second antibody or antigen binding fragment thereof; and
3) detecting Pepsinogen C in the sample by detecting the formation of the complex as defined in 2).

190. A composition comprising
a) the antibody or antigen binding fragment thereof according to item 176 or 177 or according to item 184 as dependent from item 176 or 177;
and the antibody or antigen binding fragment thereof according to item 178 or 179 or according to item 184 as dependent from item 178 or 179;
b) the polynucleotide or set of polynucleotides of item 180 as dependent from item 178 or 179 or according to item 184 as dependent from item 178 or 179;
and the polynucleotide or set of polynucleotides of item 180 as dependent from item 178 or 179 or according to item 184 as dependent from item 178 or 179;
c) the vector of item 181 as dependent from item 178 or 179 or according to item 184 as dependent from item 178 or 179;
and the vector of item 181 as dependent from item 178 or 179 or according to item 184 as dependent from item 178 or 179; or
d) the host cell according to item 182 as dependent from item 178 or 179 or according to item 184 as dependent from item 178 or 179;
and the host cell according to item 182 as dependent from item 178 or 179 or according to item 184 as dependent from item 178 or 179.

191. The kit of item 174, wherein the first antibody or antigen binding fragment comprises
(a) a VH comprising a CDR-H1 as shown in the amino acid sequence of SEQ ID NO: 3 or a variant thereof modified by one amino acid substitution; a CDR-H2 as shown in the amino acid sequence of SEQ ID NO: 4, or a variant thereof modified by one amino acid substitution; and a CDR-H3 as shown in the amino acid sequence of SEQ ID NO: 5, or a variant thereof modified by one amino acid substitution; and
a VL comprising a CDR-L1 as shown in the amino acid sequence of SEQ ID NO: 6 or a variant thereof modified by one amino acid substitution; a CDR-L2 as shown in the amino acid sequence of SEQ ID NO: 7, or a variant thereof modified by one amino acid substitution; and a CDR-L3 as shown in the amino acid sequence of SEQ ID NO: 52, or a variant thereof modified by one amino acid substitution; or
(b) a VH comprising a CDR-H1 as shown in the amino acid sequence of SEQ ID NO: 36 or a variant thereof modified by one amino acid substitution; a CDR-H2 as shown in the amino acid sequence of SEQ ID NO: 37, or a variant thereof modified by one amino acid substitution; and a CDR-H3 as shown in the amino acid sequence of SEQ ID NO: 38, or a variant thereof modified by one amino acid substitution; and
a VL comprising a CDR-L1 as shown in the amino acid sequence of SEQ ID NO: 39 or a variant thereof modified by one amino acid substitution; a CDR-L2 as shown in the amino acid sequence of SEQ ID NO: 40, or a variant thereof modified by one amino acid substitution; and a CDR-L3 as shown in the amino acid sequence of SEQ ID NO: 41, or a variant thereof modified by one amino acid substitution.

192. The kit of item 174 or 191, wherein the second antibody or antigen binding fragment thereof comprises
a VH comprising a CDR-H1 as shown in the amino acid sequence of SEQ ID NO: 11 or a variant thereof modified by one amino acid substitution; a CDR-H2 as shown in the amino acid sequence of SEQ ID NO: 12, or a variant thereof modified by one amino acid substitution; and a CDR-H3 as shown in the amino acid sequence of SEQ ID NO: 13, or a variant thereof modified by one amino acid substitution; and
VL comprising a CDR-L1 as shown in the amino acid sequence of SEQ ID NO: 14 or a variant thereof modified by one amino acid substitution; a CDR-L2 as shown in the amino acid sequence of SEQ ID NO: 15, or a variant thereof modified by one amino acid substitution; and a CDR-L3 as shown in the amino acid sequence of SEQ ID NO: 16, or a variant thereof modified by one amino acid substitution.

193. The kit of item 174, wherein the first antibody or antigen binding fragment comprises
(a) a VH comprising a CDR-H1 as shown in the amino acid sequence of SEQ ID NO: 3 or a variant thereof modified by one amino acid substitution; a CDR-H2 as shown in the amino acid sequence of SEQ ID NO: 4, or a variant thereof modified by one amino acid substitution; and a CDR-H3 as shown in the amino acid sequence of SEQ ID NO: 5, or a variant thereof modified by one amino acid substitution; and
a VL comprising a CDR-L1 as shown in the amino acid sequence of SEQ ID NO: 6 or a variant thereof modified by one amino acid substitution; a CDR-L2 as shown in the amino acid sequence of SEQ ID NO: 7, or a variant thereof modified by one amino acid substitution; and a CDR-L3 as shown in the amino acid sequence of SEQ ID NO: 52, or a variant thereof modified by one amino acid substitution; or
(b) a VH comprising a CDR-H1 as shown in the amino acid sequence of SEQ ID NO: 36 or a variant thereof modified by one amino acid substitution; a CDR-H2 as shown in the amino acid sequence of SEQ ID NO: 37, or a variant thereof modified by one amino acid substitution; and a CDR-H3 as shown in the amino acid sequence of SEQ ID NO: 38, or a variant thereof modified by one amino acid substitution; and
a VL comprising a CDR-L1 as shown in the amino acid sequence of SEQ ID NO: 39 or a variant thereof modified by one amino acid substitution; a CDR-L2 as shown in the amino acid sequence of SEQ ID NO: 40, or a variant thereof modified by one amino acid substitution; and a CDR-L3 as shown in the amino acid sequence of SEQ ID NO: 41, or a variant thereof modified by one amino acid substitution; and
wherein the second antibody, or antigen binding fragment thereof, comprises
(c) a VH comprising a CDR-H1 as shown in the amino acid sequence of SEQ ID NO: 11 or a variant thereof modified by one amino acid substitution; a CDR-H2 as shown in the amino acid sequence of SEQ ID NO: 12, or a variant thereof modified by one amino acid substitution; and a CDR-H3 as shown in the amino acid sequence of SEQ ID NO: 13, or a variant thereof modified by one amino acid substitution; and
VL comprising a CDR-L1 as shown in the amino acid sequence of SEQ ID NO: 14 or a variant thereof modified by one amino acid substitution; a CDR-L2 as shown in the amino acid sequence of SEQ ID NO: 15, or a variant thereof modified by one amino acid substitution; and a CDR-L3 as shown in the amino acid sequence of SEQ ID NO: 16, or a variant thereof modified by one amino acid substitution.

194. An in vitro method for detecting and/or quantifying Pepsinogen C in a sample, comprising the following steps:
1) contacting a sample comprising Pepsinogen C with a first antibody, or antigen binding fragment thereof, and a second antibody, or antigen binding fragment thereof, wherein
(a) the first antibody is
(i) the antibody or antigen binding fragment thereof according to any one of items 1-16 or 45 or according to any one of items 29 to 38 as dependent from any one of items 1 to 16; or
(ii) is the antibody or antigen binding fragment thereof according to according any one of items 17-28 or 45 or according to any one of items 29 to 38 as dependent from any one of items 17 to 28; and
(b) the second antibody does not compete with the first antibody for specifically binding to Pepsinogen C;
2) incubating the resulting mixture of 1) to form a complex comprising Pepsinogen C and said first antibody or antigen binding fragment thereof and said second antibody or antigen binding fragment thereof, and
3) detecting Pepsinogen C in the sample by detecting the formation of the complex as defined in 2), and
(c) optionally quantifying the amount of Pepsinogen C in the sample.

195. An in vitro method for detecting and/or quantifying Pepsinogen C in a sample, comprising the following steps:
1) contacting a sample comprising Pepsinogen C with a first antibody, or antigen binding fragment thereof, and a second antibody, or antigen binding fragment thereof, wherein
(a) the first antibody is
(i) the antibody or antigen binding fragment thereof according to any one of items 72 to 114 or according to any one of items 131-140 or 147 as dependent from any one of items 72 to 114; or
(ii) is the antibody or antigen binding fragment thereof according to any one of items 115 to 130 or according to any one of items 131-140 or 147 as dependent from any one of items 115 to 130;
(b) the second antibody does not compete with the first antibody for specifically binding to Pepsinogen C; and
2) incubating the resulting mixture of 1) to form a complex comprising Pepsinogen C and said first antibody or antigen binding fragment thereof and said second antibody or antigen binding fragment thereof; and
3) detecting Pepsinogen C in the sample by detecting the formation of the complex as defined in 2), and
(c) optionally quantifying the amount of Pepsinogen C in the sample.

196. An in vitro method for detecting and/or quantifying Pepsinogen C in a sample, comprising
(a) contacting a sample comprising Pepsinogen C with the antibody, or antigen binding fragment thereof, according to any one of items 1-38 or 45, thereby forming a complex comprising Pepsinogen C and said antibody, or antigen binding fragment thereof, and
(b) detecting the complex formed in step (a), thereby detecting Pepsinogen C in said sample, and
(c) optionally quantifying the amount of Pepsinogen C in said sample.

197. An in vitro method for detecting and/or quantifying Pepsinogen C in a sample, comprising
(a) contacting a sample comprising Pepsinogen C with the antibody, or antigen binding fragment thereof, according to any one of items 72-140 or 147, thereby forming a complex comprising Pepsinogen C and said antibody, or antigen binding fragment thereof, and
(b) detecting the complex formed in step (a), thereby detecting Pepsinogen C in said sample, and
(c) optionally quantifying the amount of Pepsinogen C in said sample.

198. A kit comprising a first antibody or antigen binding fragment thereof and a second antibody or antigen binding fragment thereof,
a) wherein the first antibody or antigen binding fragment thereof is
an antibody or antigen binding fragment thereof that specifically binds to Pepsinogen C comprising
(i) a VH comprising a CDR-H1 as shown in the amino acid sequence of SEQ ID NO: 3 or a variant thereof modified by one amino acid substitution; a CDR-H2 as shown in the amino acid sequence of SEQ ID NO: 4, or a variant thereof modified by one amino acid substitution; and a CDR-H3 as shown in the amino acid sequence of SEQ ID NO: 5, or a variant thereof modified by one amino acid substitution; and
a VL comprising a CDR-L1 as shown in the amino acid sequence of SEQ ID NO: 6 or a variant thereof modified by one amino acid substitution; a CDR-L2 as shown in the amino acid sequence of SEQ ID NO: 7, or a variant thereof modified by one amino acid substitution; and a CDR-L3 as shown in the amino acid sequence of SEQ ID NO: 52, or a variant thereof modified by one amino acid substitution; or
(ii) a VH comprising a CDR-H1 as shown in the amino acid sequence of SEQ ID NO: 36 or a variant thereof modified by one amino acid substitution; a CDR-H2 as shown in the amino acid sequence of SEQ ID NO: 37, or a variant thereof modified by one amino acid substitution; and a CDR-H3 as shown in the amino acid sequence of SEQ ID NO: 38, or a variant thereof modified by one amino acid substitution; and
a VL comprising a CDR-L1 as shown in the amino acid sequence of SEQ ID NO: 39 or a variant thereof modified by one amino acid substitution; a CDR-L2 as shown in the amino acid sequence of SEQ ID NO: 40, or a variant thereof modified by one amino acid substitution; and a CDR-L3 as shown in the amino acid sequence of SEQ ID NO: 41, or a variant thereof modified by one amino acid substitution; and
(b) wherein the second antibody or antigen binding fragment thereof is
an antibody or antigen binding fragment thereof that specifically binds to Pepsinogen C comprising
(c) a VH comprising a CDR-H1 as shown in the amino acid sequence of SEQ ID NO: 11 or a variant thereof modified by one amino acid substitution; a CDR-H2 as shown in the amino acid sequence of SEQ ID NO: 12, or a variant thereof modified by one amino acid substitution; and a CDR-H3 as shown in the amino acid sequence of SEQ ID NO: 13, or a variant thereof modified by one amino acid substitution; and
VL comprising a CDR-L1 as shown in the amino acid sequence of SEQ ID NO: 14 or a variant thereof modified by one amino acid substitution; a CDR-L2 as shown in the amino acid sequence of SEQ ID NO: 15, or a variant thereof modified by one amino acid substitution; and a CDR-L3 as shown in the amino acid sequence of SEQ ID NO: 16, or a variant thereof modified by one amino acid substitution.

wherein the second antibody or antigen binding fragments thereof does not compete for binding to Pepsinogen C with the first antibody or antigen binding fragment thereof.

Aspects of the invention are demonstrated by the following non-limiting examples. The following examples and figures are provided to aid the understanding of the present invention, the true scope of which is set forth in the appended claims. It is understood that modifications can be made in the procedures set forth without departing from the spirit of the invention.

### EXAMPLES

The following examples are provided to aid the understanding of the present invention, the true scope of which is set forth in the appended claims. It is understood that modifications can be made in the procedures set forth without departing from the spirit.

### Example 1: Immunogens and Screening Reagents

For native recombinant expression, the coding sequence of Pepsinogen C (PepC) (SEQ ID NO: 62) and Pepsinogen A5 (SEQ ID NO: 63) equipped with a C terminal Ala-Gly3-Ser-His8-tag was subcloned via SalI/BamHI into the eukaryotic expression vector pM1MT (Roche Molecular Biochemicals).

For transient gene expression in human embryonic kidney (HEK) 293 cells, the FreeStyle™ 293-F expression system (Thermo Fischer Scientific) was used: Cultures at ~2×10⁶ cells/ml were transfected with the respective expression plasmid at a concentration of 0.5 mg/l cell culture complexed by the PEIpro® (Polyplus) transfection reagent (Merck Millipore) according to the manufacturer's guidelines. Three hours post-transfection, the histone deacetylase inhibitor valproic acid was added to 4 mM to boost protein production (Backliwal et al. 2008. Valproic acid: A viable alternative to sodium butyrate for enhancing protein expression in mammalian cell cultures. Biotechnology and Bioengineering 101:182-189). Each day, the culture was supplemented with 6% (v/v) of a soybean peptone hydrolysate-based feed. Seven days post-transfection, the culture supernatant was collected, cleared from cell debris by centrifugation and stored at −80 °C for purification. The protein is secreted into the cell culture supernatant and purified by immobilized metal chelate affinity chromatography (IMAC) followed by size exclusion chromatography (SEC).

>Pepsinogen C with tag
(SEQ ID NO: 62)
MKWMVVVLVCLQLLEAAVVKVPLKKFKSIRETMKEKGLLGEFLRTHKYDP
AWKYRFGDLSVTYEPMAYMDAAYFGEISIGTPPQNFLVLFDTGSSNLWVP
SVYCQSQACTSHSRFNPSESSTYSTNGQTFSLQYGSGSLTGFFGYDTLTV
QSIQVPNQEFGLSENEPGTNFVYAQFDGIMGLAYPALSVDEATTAMQGMV
QEGALTSPVFSVYLSNQQGSSGGAVVFGGVDSSLYTGQIYWAPVTQELYW
QIGIEEFLIGGQASGWCSEGCQAIVDTGTSLLTVPQQYMSALLQATGAQE
DEYGQFLVNCNSIQNLPSLTFIINGVEFPLPPSSYILSNNGYCTVGVEPT
YLSSQNGQPLWILGDVFLRSYYSVYDLGNNRVGFATAAAAGGGSHHHHHH
HH
>Pepsinogen A5 with tag
(SEQ ID NO: 63)
MKWLLLLGLVALSECIMYKVPLIRKKSLRRTLSERGLLKDFLKKHNLNPA
RKYFPQWEAPTLVDEQPLENYLDMEYFGTIGIGTPAQDFTVVFDTGSSNL
WVPSVYCSSLACTNHNRFNPEDSSTYQSTSETVSITYGTGSMTGILGYDT
VQVGGISDTNQIFGLSETEPGSFLYYAPFDGILGLAYPSISSSGATPVFD
NIWNQGLVSQDLFSVYLSADDKSGSVVIFGGIDSSYYTGSLNWVPVTVEG
YWQITVDSITMNGETIACAEGCQAIVDTGTSLLTGPTSPIANIQSDIGAS
ENSDGDMVVSCSAISSLPDIVFTINGVQYPVPPSAYILQSEGSCISGFQG
MNVPTESGELWILGDVFIRQYFTVFDRANNQVGLAPVAAAGGGSHHHHHH
HH

### Example 2: Purification of Antigens

The culture supernatant was centrifuged to remove the cells and dialyzed against 50 mM Hepes pH 7.0, 300 mM NaCl. The dialysate was applied to a Complete His-Tag Purification Resin column (Roche). The column was eluted with 50 mM Hepes pH 7.0, 300 mM NaCl and 500 mM imidazole. The protein eluted with 500 mM imidazole was dialyzed against 50 mM Hepes pH 7.0, 50 mM NaCl with 6.5% sucrose. The dialysed sample was applied to a Superdex 75 prep grade column (Cytiva). The protein fractions corresponding to monomeric pepsinogen were pooled, aliquoted and stored at −80 °C. The proteins were characterized by SEC-MALS HPLC and SDS-PAGE.

### Example 3: Immunization Strategy

To generate structural antibodies against Pepsinogen C (PepC), mice (5×Balb/C, 5×NMRI, inbreeding strains) were immunized with recombinant protein (PepC-Ala-Gly3-Ser-His8; see chapter “Immunogen”).

After immunization, spleens of the immunized animals were prepared and fused to myeloma-cells (P3X63Ag8.653).

### Example 4: Screening/Hit-ELISA (Primary Cultures)

### Screening Reagents:

Purified Pepsinogen C was biotinylated using standard NHS-chemistry with an activated ester of Biotin Biotin-DDS (i.e. Biotin-Diaminodioxaoctane-succinimidylsuberate), and purified using size exclusion chromatography.

### Hit-ELISA:

The hybridoma-cells resulting from 3 were cultivated and supernatants were tested by ELISA (see FIG. 1) for their reactivity against recombinant PepC.

More than 600 positive primary cultures were utilized to generate monoclonal anti-PepC-antibody expressing cells. After cultivation, supernatants were tested again for their reactivity against PepC (see FIG. 1).

From the tested clones, 14 candidates were isolated, cultivated in larger amounts (CELLine) and transferred to Biacore as well as test development (Elecsys) for further investigations.

### Example 5: Kinetic Screening/Surpace Plasmon Resonance (SPR) (Biacore) (Primary Cultures)

### SPR Analysis

All SPR experiments were conducted with recombinant Pepsinogen A (PGA) with a molecular mass 41.8 kDa, and Pepsinogen C (PGC), molecular mass 42.1 kDa.

The experiments were performed at on GE Healthcare BIAcore™ 8K+ and B4000 instruments.

All interactions were analyzed at 37 °C using the system buffer HBS-ET+pH 7.4, 10 mM HEPES, 150 mM NaCl, 3 mM EDTA, 0.05% (w/v) Tween 20, pH 7.4. The system buffer was supplemented with 1 mg/mL Carboxymethyldextran (Fluka) and was used as sample buffer.

### Functionalizing the Sensor

A Biacore CM5 Series S was functionalized at 25 °C with a mouse species-specific antibody capture system. A polyclonal rabbit anti-mouse IgG Fc capture antibody RAMFcγ (Cat. No. 315-005-046, Jackson Immuno Research) or polyclonal goat anti mouse IgG Fc capture antibody (Cat. No. 115-005-071, Jackson Immuno Research) was amine coupled using EDC/NHS chemistry according to the manufacturer's instructions. Finally, ligand densities between 11500-13500 RU (B4000) resp. 10000-11400 RU (8K+) were obtained.

Mouse antibodies (IgG 150 kDa) solutions were diluted in sample buffer and were injected at 5 pl/min or 10 μL/min for 2 minutes. The antibody Capture Level (CL) in resonance units (RU) was monitored.

### Kinetic-Screening

150 nM analytes PGA or PGC were injected to the surface-displayed antibodies. The analyte association phases were monitored for 5 minutes. The antibody/pepsinogen protein complex dissociation phases were monitored for 5 minutes. After each measurement cycle the capture systems were regenerated by two subsequent injections of 10 mM Glycine buffer pH 1.7 at 20 L/min for 60 seconds resp. a single injection of 10 mM Glycine buffer pH 2.0, followed by two subsequent injections of pH 2.25 with each 60 seconds at 20 μL/min.

The kinetic signatures were monitored by the BIAcore™ B4000 Control SW V1.1 resp. 8K Control-SW V4.0.8.20368 and evaluated via Evaluation SW V1.1 resp. by the BIAcore™ Insight Evaluation SW V4.0.8.20368

Kinetic data was interpreted by report point evaluations. Two report points, the recorded response signal shortly before the end of the protein analyte injection, analyte Binding Late (BL), and the signal shortly before the end of the dissociation phase, Stability Late (SL), were used to compare the antibody/antigen complex stabilities.

The dissociation rate constant k_{d} (s⁻¹) was calculated according to a dissociation fitting Langmuir model and the antibody/antigen complex half-life was calculated in minutes according to the formula t_{2 diss}=ln(2)/(k_{d}*60). The Molar Ratio, the binding stoichiometry was calculated by the formula MR=BL(antigen)*MW(antibody)/(MW(antigen)*CL (antibody)).

466 antibodies from culture supernatants were analyzed. Candidates with specific binding for PGC and no cross reactivity with PGA were identified. Further, the obtained sensorgrams of the identified candidates were analyzed by visual inspection for fastest complex formation into Ab-equilibrium and stable complex half-life times. Antibodies with to slow complex formation were deselected.

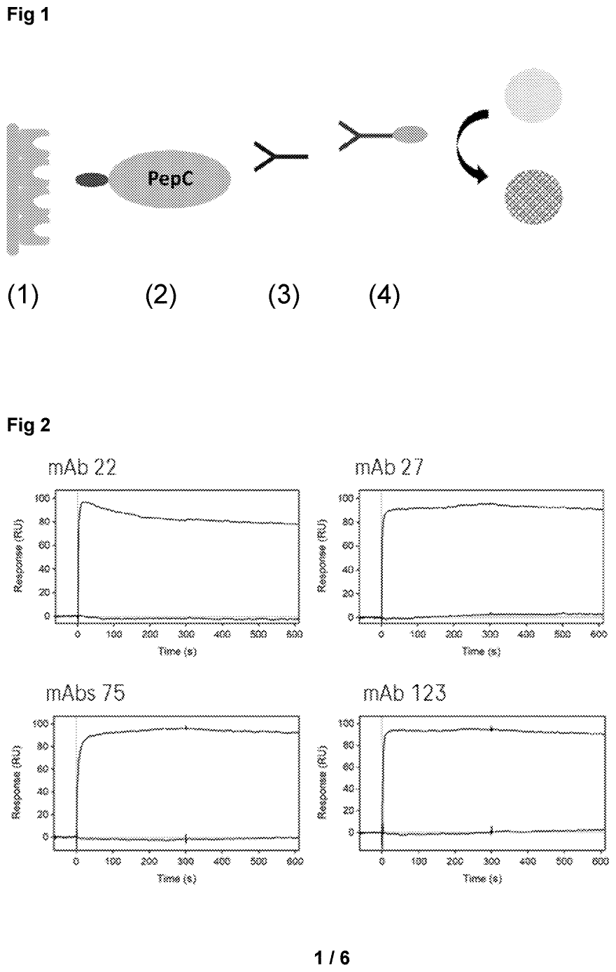

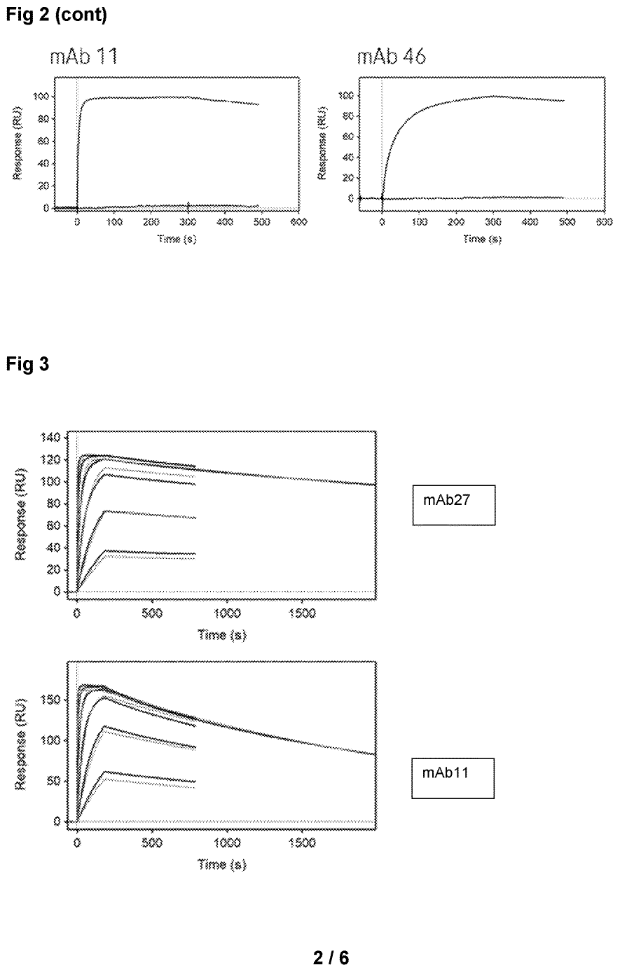

Results are shown in FIG. 2, which show the kinetic screening with exemplary kinetic signatures of antibody/Pepsinogen interactions. Shown is the binding to 150 nM PGA (bottom sensorgram) resp. PGC (top sensorgram) as overlaid normalized sensorgrams for antibody interactions with specificity for PGC (top sensorgram) and no cross reactivity with PGA (bottom sensorgram).

### Example 6: Production of IgG (Cell Line)

Selected Hybridoma clones were taken into culture in a small scale dialysis-fermentation unit, and grown for production of IgG for several weeks with repeated change of dialysis media. The cell culture supernatant was harvested, and IgG was purified using standard affinity chromatography methods using Mab Select Sure.

### Example 7: Production/Derivatisation of IgGs

### Generation of Anti-Pepsinogen-IgG Ruthenium Label Conjugates:

Purified anti-Pepsinogen-IgG antibodies were labelled with an N-Hydroxy-succinimide activated ester of sulfo Bispiridyl-Ruthenium according to standard laboratory procedures. Shortly, to a solution of IgG (5.0 mg, 1.11 mL in 100 mM KPO₄ pH 8.4), 56 μL solution of Hy-BPRU NHS ESTER (Ruthenate(3-), bis(2,2′-bipyridine-κN1,κN1′)[N-[4-(4′-methyl[2,2′-bipyridin]-4-yl-κN1,κN1′)-1-oxobutyl]-β-alanyl-L-α-glutamyl-L-α-glutamyl-N6-[8-[(2,5-dioxo-1-pyrrolidinyl)oxy]-1,8-dioxooctyl]-L-lysinato(3-)]-, (OC-6-33)-(9CI) (BPRu-UEEK-DSS), CAS Nr. 406218-59-5; 4.24 mg/ml in DMSO) are added (i.e. stoichiometry of 4.0 mol Ruthenium-NHS-ester per mol IgG). After 120 min stirring at room temperature, the derivatization was stopped by addition of Lysine at 10 mM final concentration. pH was adjusted to pH 7.5 with saturated KH₂PO₄, and the reaction mixture was dialysed against 50 mM KPO₄/0.15 M KCl/2% saccharose, pH 7.5 overnight. For removal of aggregates and remaining hydrolized Ruthenium ester, appropriate fractions were collected from Size-exclusion chromatography on a Superdex 200 HR 10/30 column (GE Healthcare Life Sciences). After addition of sucrose to a final concentration of 6.5% (w/v), preparations were filtered over 0.45 μm PVDF syringe filters (Acrodisk Pall Life Sciences Corp.) and stored frozen at −80 °C.

### Generation of Anti-Pepsinogen-IgG Biotin Label Conjugates:

Same procedure as described above for Ruthenium label with the exception that instead of 56 μL solution of Hy-BPRU NHS ESTER (Ruthenate(3-), bis(2,2′-bipyridine-κN1,κN1′)[N-[4-(4′-methyl[2,2′-bipyridin]-4-yl-κN1,κN1′)-1-oxobutyl]-β-alanyl-L-α-glutamyl-L-α-glutamyl-N6-[8-[(2,5-dioxo-1-pyrrolidinyl)oxy]-1,8-dioxooctyl]-L-lysinato(3-)]-, (OC-6-33)-(9CI) (BPRu-UEEK-DSS), CAS Nr. 406218-59-5; 4.24 mg/ml in DMSO) 56 μL solution of Biotin-DDS ESTER (2,5-dioxopyrrolidin-1-yl 5,16-dioxo-1-((3aS,4S,6aR)-2-oxohexahydro-1H-thieno[3,4-d]imidazol-4-yl)-9,12-dioxa-6,15-diazatricosan-23-oate, CAS Nr. 267898-87-3; 1.22 mg/ml in DMSO) were added in the first step.

### Example 8: Functional Assessment of Anti-Pepsinogen Conjugates

The assay principle of the sandwich immunological assay using an automated Elecsys Immuno-Analyzer (Elecsys® Cobas® e411) is summarized as follows: The total incubation time required for the assay was 18 minutes: 1st incubation (9 min): 15 μL of Pepsinogen C containing sample, 75 μL of ruthenylated monoclonal Pepsinogen C-specific IgG antibody and 75 μL of a solution of biotinylated monoclonal Pepsinogen C-specific IgG antibody was incubated and formed a sandwich complex. 2nd incubation (9 min): 35 μL of Streptavidin-coated microparticles were added to the mixture of the first incubation step. During this second incubation the sandwich complex binds to the Streptavidin coated magnetic bead/solid phase via interaction of biotin and streptavidin. The reaction mixture was aspirated into the measuring cell where the microparticles were magnetically captured onto the surface of the electrode. Unbound substances were then removed with ProCell #11662988122 (Roche Diagnostics GmbH Germany). Application of a voltage to the electrode then induced electro-chemiluminescence-based emission of light which was measured by a photomultiplier.

A plot of ECL signal in counts vs. concentration of Pepsinogen C results in atypical curve with raising signal with rising Pepsinogen concentration.

TABLE 1
Table showing signals of different mAb sandwich pairs

| | Clone | mAb<PGC>-Ruthenium-Conjugate | | | | | |
|---|---|---|---|---|---|---|---|
| | ID | mAb22 | mAb27 | mAb75 | mAb123 | mAb11 | mAb46 |
| mAb< | mAb22 | 5182 | 5269 | 3791 | 4865 | 1692560 | 504665 |
| PGC> | mAb27 | 5157 | 5239 | 3720 | 4847 | 1680490 | 498294 |
| Biotin- | mAb75 | 4750 | 4982 | 4724 | 4707 | 1474680 | 791804 |
| | mAb123 | 5081 | 5231 | 3758 | 4805 | 1648340 | 450957 |
| | mAb11 | 1566810 | 1595680 | 1348190 | 1497670 | 16735 | 752613 |
| | mAb46 | 516365 | 533605 | 934515 | 452082 | 1019910 | 5587 |

Good sandwich pairs
No sandwich
Homologous pairs

Based on the sandwich-compatibility it was concluded, that at least 3 independent epitopes are recognized by the antibodies which can be grouped as follows:

TABLE 2
Identified epitopes of Pepsinogen C. Epitopes were
identified based on the sandwich-compatibility.

| | | Sandwich/immunological |
|---|---|---|
| Epitope | Antibody | assay |
| A | mAb 22, mAb 27, | all mAbs can form |
| | mAb 75, mAb 123 | sandwich with mAbs |
| | | recognizing epitope B or C |
| B | mAb 11 | mAb can form sandwich |
| | | with mAbs recognizing |
| | | Epitope A or C |
| C | mAb 46 | mAb can form sandwich |
| | | with mAbs recognizing |
| | | Epitope A or B |

Linear peptide mapping did not identify any linear epitopes (data not shown), thus the identified epitopes are all conformational epitopes.

### Example 9: Kinetic Screening/Biacore (Clones)

If not stated otherwise, the experiments for determining the kinetic constants and resulting affinities were performed as stated under kinetic screening (Example 5; Kinetic screening):

Instead of a single concentration, multi cycle kinetic experiments were performed for PGC. A series with increasing Pepsinogen C concentrations 1.2-450 nM, dilution factor 3, were injected to the pre-captured antibodies.

The analyte association phases were monitored for 3 minutes. The antibody/Pepsinogen C protein complex dissociation phases were monitored for 10 minutes. Additionally, the 50 nM concentration was monitored with 30 minutes dissociation time.

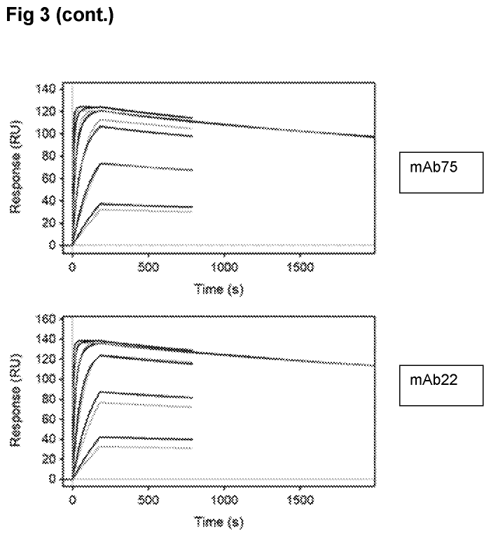

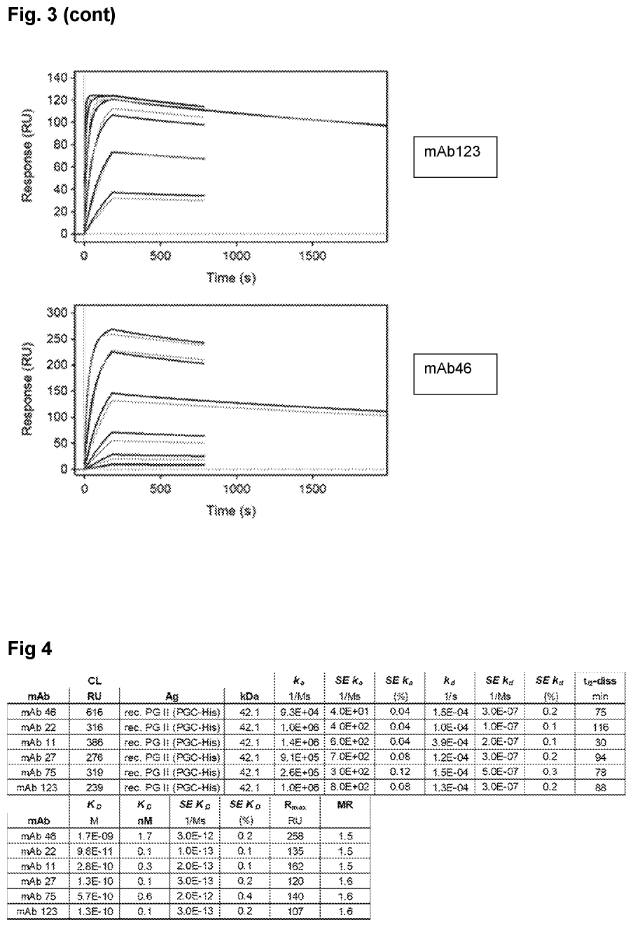

FIG. 3 shows the kinetic signatures for mAb 27, mAb 75, mAb 123, mAb 11, mAb 22 and mAb 46 binding to PGC. Interactions are overlaid with a Langmuir 1:1 binding model (grey). The kinetic parameters determined for mAB mAb 46, mAb 22, mAb 11, mAb 27, mAb 75 and mAb 123 are shown in FIG. 4. The highest affinity to PGC has mAb 22 (KD E-11), whereas mAb 46 has the lowest affinity to PGC (KD E-9). The further antibodies tested all show similar K_{D} in between (K_{D} E-10).

From Elecsys Data: mAb 11 can form immuno-complex with all further analyzed candidates, best partner is mAb 22 (Bi), lowest counts are generated with mAb 46 which also displayed lowest affinity. So Biacore and Elecsys Data are consistent.

### Example 10: Sequence Alignment

The sequences of the VL and the VH of the antibodies mAB 22, 27, 75, 123 and 11 were aligned and the CDRs are boxed (FIG. 5). The CDRs are determined according to Kabat et al.,

### Example 11: Assay Development

A PGC Elecsys Assay was developed using two antibodies for PGC binding different epitopes. The Assay has a very low sample volume of 12 pl (other assays on the market require at least 25 pl) allowing for more tests from the same sample. The surprising high affinity of the antibodies translates into a very good precision at low concentrations. Therefore the Limit of Quantification (LoQ) has been determined.

The LoQ is defined as the lowest amount of analyte in a sample that can be accurately quantitated with an intermediate precision CV of ≤20%.

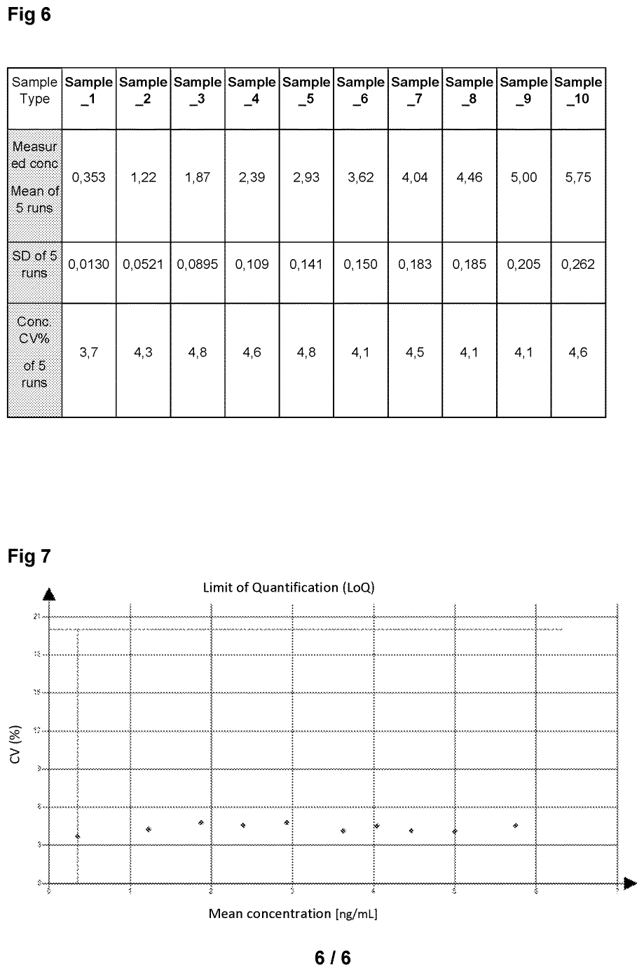

For Limit of Quantitation 10 human serum samples were measured over 5 runs in 2 days in 5 replicates on 1 analyzer. This gave an intermediate precision CV of ≤20% (FIG. 6 and FIG. 7).

The result for the LoQ was 0,353 ng/mL at 20% CV.

TABLE 3
SEQ ID NOs of the present invention including names and amino acid sequences.
In the sequences X₁ is Ala or Gly, X₂ is Ile or Met, X₃ is Phe or Ile, X₄
is Asn or Thr, X₅ is Gly or Ala, X₆ is Lys or Asn and X₇ is Val or Ile.

| SEQ | | |
|---|---|---|
| ID | | |
| NO | Name | Sequence |
| 1 | VH | QIQLVQSGPELKKPGETVKISCKASGYTFTNYGMN |
| | mAb22 | WVKQAPGKGLKWIGWINTYTGDPTYADDFKGRFA |
| | | FSLETSASNGYLQIDNLKKEDTATYFCARRDIFAM |
| | | DYWGQGTSVTVSS |
| 2 | VL | DIQMTQTTSSLSASLGDRVTITCRASQDIMKYLHW |
| | mAb22 | YQQKPDGTIKLLIYYTSRLHSGVPSRFSGSGSGTDY |
| | | SLTISNLEQEDIATYFCQQDVKYPWTFGGGTKLEIK |
| 3 | CDR-H1 | NYGMN |
| | mAb 22 | |
| | and | |
| | 22/27/1 | |
| | 23 | |
| 4 | CDR-H2 | WINTYTGDPTYADDFKG |
| | mAb | |
| | 22 and | |
| | 22/27/1 | |
| | 23 | |
| 5 | CDR- | RDIFAMDY |
| | H3 | |
| | mAb | |
| | 22 and | |
| | 22/27/1 | |
| | 23 | |
| 6 | CDR- | RASQDIMKYLH |
| | L1 | |
| | mAb | |
| | 22 and | |
| | 22/27/1 | |
| | 23 | |
| 7 | CDR- | YTSRLHS |
| | L2 | |
| | mAb | |
| | 22 and | |
| | 22/27/1 | |
| | 23 | |
| 8 | CDR- | QQDVKYPWT |
| | L3 | |
| | mAb | |
| | 22 | |
| 9 | VH | QVQLQQPGTELVKPGASVKLSCKASGYTFTNYWM |
| | mAb | HWVKQRPGQGLEWIGNINPFNGATNYNDNFKTKA |
| | 11 | TLTVDKSSSTAYMHLSSLTSEDSAVYYCARYDYSK |
| | | YYAMDYWGQGTSVTVSS |
| 10 | VL | ENVLTQSPAIMAASPGDKVTMTCSASSSINSAHFH |
| | mAb | WYQQKPGTSPKLWIYRTSNLASGVPARFSGRGSGT |
| | 11 | SYSLTISSMEAADAATYYCQQWSDYPWTFGGGTK |
| | | LEIR |
| 11 | CDR- | NYWMH |
| | H1 | |
| | mAb | |
| | 11 | |
| 12 | CDR- | NINPFNGATNYNDNFKT |
| | H2 | |
| | mAb | |
| | 11 | |
| 13 | CDR- | YDYSKYYAMDY |
| | H3 | |
| | mAb | |
| | 11 | |
| 14 | CDR- | SASSSINSAHFH |
| | L1 | |
| | mAb | |
| | 11 | |
| 15 | CDR- | RTSNLAS |
| | L2 | |
| | mAb | |
| | 11 | |
| 16 | CDR- | QQWSDYPWT |
| | L3 | |
| | mAb | |
| | 11 | |
| 17 | Pepsin- | MKWMVVVLVCLQLLEAAVVKVPLKKFKSIRETM |
| | ogen C | KEKGLLGEFLRTHKYDPAWKYRFGDLSVTYEPMA |
| | | YMDAAYFGEISIGTPPQNFLVLFDTGSSNLWVPSVY |
| | | CQSQACTSHSRFNPSESSTYSTNGQTFSLQYGSGSL |
| | | TGFFGYDTLTVQSIQVPNQEFGLSENEPGTNFVYAQ |
| | | FDGIMGLAYPALSVDEATTAMQGMVQEGALTSPV |
| | | FSVYLSNQQGSSGGAVVFGGVDSSLYTGQIYWAPV |
| | | TQELYWQIGIEEFLIGGQASGWCSEGCQAIVDTGTS |
| | | LLTVPQQYMSALLQATGAQEDEYGQFLVNCNSIQ |
| | | NLPSLTFIINGVEFPLPPSSYILSNNGYCTVGVEPTYL |
| | | SSQNGQPLWILGDVFLRSYYSVYDLGNNRVGFATA |
| | | A |
| 18 | FW-L1 | DIQMTQTTSSLSASLGDRVTITC |
| | mAb | |
| | 22 and | |
| | 22/27/1 | |
| | 23 | |
| 19 | FW-L2 | WYQQKPDGTIKLLIY |
| | mAb | |
| | 22 | |
| 20 | FW-L3 | SGVPSRFSGSGSGTDYSLTISNLEQEDIATYFC |
| | mAb | |
| | 22 and | |
| | 22/27/1 | |
| | 23 | |
| 21 | FW- | FGGGTKLEIK |
| | L4 | |
| | mAb | |
| | 22 and | |
| | 22/27/1 | |
| | 23 | |
| 22 | FW- | QIQLVQSGPELKKPGETVKISCKASGYTFT |
| | H1 | |
| | mAb | |
| | 22 | |
| 23 | FW- | WVKQAPGKGLKWIG |
| | H2 | |
| | mAb | |
| | 22 | |
| 24 | FW-H3 | RFAFSLETSASNGYLQIDNLKKEDTATYFCAR |
| | mAb | |
| | 22 | |
| 25 | FW-H4 | WGQGTSVTVSS |
| | mAb | |
| | 22 and | |
| | 22/27/1 | |
| | 23 | |
| 26 | FW-L1 | ENVLTQSPAIMAASPGDKVTMTC |
| | mAb | |
| | 11 | |
| 27 | FW-L2 | WYQQKPGTSPKLWIY |
| | mAb | |
| | 11 | |
| 28 | FW-L3 | GVPARFSGRGSGTSYSLTISSMEAADAATYYC |
| | mAb | |
| | 11 | |
| 29 | FW-L4 | FGGGTKLEIR |
| | mAb | |
| | 11 | |
| 30 | FW- | QVQLQQPGTELVKPGASVKLSCKASGYTFT |
| | H1 | |
| | mAb | |
| | 11 | |
| 31 | FW-H2 | WVKQRPGQGLEWIG |
| | mAb | |
| | 11 | |
| 32 | FW-H3 | KATLTVDKSSSTAYMHLSSLTSEDSAVYYCAR |
| | mAb | |
| | 11 | |
| 33 | FW-H4 | WGQGTSVTVSS |
| | mAb | |
| | 11 | |
| 34 | VH | QIQLVQSGPELKKPGETVKISCKASGYTFTNYGMN |
| | mAb | WVKQAPGKGLKWMGLINTYTRGPTFADDFKGRFA |
| | 75 | FSVETSANTAYLQINNLKTEDTATYFCARRGEYYG |
| | | SSYVDYWGQGTTLTVSS |
| 35 | VL | DIQMTQITSSLSASLGDRVTISCRASQDIMHFLNWY |
| | mAb | QQKPDGTVKLLIYYTSRLHSGVPSRFSGSGSGTDYS |
| | 75 | LTITNLEPEDIATYYCQQHSNLPPTFGGGTKLEIK |
| 36 | CDR- | NYGMN |
| | H1 | |
| | mAb | |
| | 75 | |
| 37 | CDR- | LINTYTRGPTFADDFKG |
| | H2 | |
| | mAb | |
| | 75 | |
| 38 | CDR- | RGEYYGSSYVDY |
| | H3 | |
| | mAb | |
| | 75 | |
| 39 | CDR- | RASQDIMHFLN |
| | L1 | |
| | mAb | |
| | 75 | |
| 40 | CDR- | YTSRLHS |
| | L2 | |
| | mAb | |
| | 75 | |
| 41 | CDR- | QQHSNLPPT |
| | L3 | |
| | mAb | |
| | 75 | |
| 42 | FW-L1 | DIQMTQITSSLSASLGDRVTISC |
| | mAb | |
| | 75 | |
| 43 | FW-L2 | WYQQKPDGTVKLLIY |
| | mAb | |
| | 75 | |
| 44 | FW-L3 | GVPSRFSGSGSGTDYSLTITNLEPEDIATYYC |
| | mAb | |
| | 75 | |
| 45 | FW-L4 | FGGGTKLEIK |
| | mAb | |
| | 75 | |
| 46 | FW-H1 | QIQLVQSGPELKKPGETVKISCKASGYTFT |
| | mAb | |
| | 75 | |
| 47 | FW-H2 | WVKQAPGKGLKWMG |
| | mAb | |
| | 75 | |
| 48 | FW- | RFAFSVETSANTAYLQINNLKTEDTATYFCAR |
| | H3 | |
| | mAb | |
| | 75 | |
| 49 | FW-H4 | WGQGTTLTVSS |
| | mAb | |
| | 75 | |
| 50 | VH | QIQLVQSGPELKKPGETVKISCKX1SGYTFTNYGMN |
| | 22/27/1 | WVKQAPGKGLKWX2GWINTYTGDPTYADDFKGR |
| | 23 | X₃AFSLETSASX₄X₅YLQIDNLKX₆EDTATYFCARRDI |
| | | FAMDYWGQGTSVTVSS |
| 51 | VL | DIQMTQTTSSLSASLGDRVTITCRASQDIMKYLHW |
| | 22/27/1 | YQQKPDGTX₇KLLX₃YYTSRLHSGVPSRFSGSGSGT |
| | 23 | DYSLTISNLEQEDIATYFCQQDX₇KYPWTFGGGTKL |
| | | EIK |
| 52 | CDR- | QQDX₇KYPWT |
| | L3 | |
| | 22/27/1 | |
| | 23 | |
| 53 | FW-H1 | QIQLVQSGPELKKPGETVKISCKX₁SGYTFT |
| | 22/27/1 | |
| | 23 | |
| 54 | FW-H2 | WVKQAPGKGLKWX₂G |
| | 22/27/1 | |
| | 23 | |
| 55 | FW- | RX₃AFSLETSASX₄X₅YLQIDNLKX₆EDTATY |
| | H3 | |
| | 22/27/1 | |
| | 23 | |
| 56 | FW-L2 | WYQQKPDGTX₇KLLX₃Y |
| | 22/27/1 | |
| | 23 | |
| 57 | CDR- | QQDIKYPWT |
| | L3 | |
| | mAb | |
| | 123 | |
| 58 | VH | QIQLVQSGPELKKPGETVKISCKGSGYTFTNYGMN |
| | mAb | WVKQAPGKGLKWMGWINTYTGDPTYADDFKGRI |
| | 123 | AFSLETSASTAYLQIDNLKNEDTATYFCARRDIFAM |
| | | DYWGQGTSVTVSS |
| 59 | VL | DIQMTQTTSSLSASLGDRVTITCRASQDIMKYLHW |
| | mAb | YQQKPDGTVKLLIYYTSRLHSGVPSRFSGSGSGTD |
| | 123 | YSLTISNLEQEDIATYFCQQDIKYPWTFGGGTKLEI |
| | | K |
| 60 | VH | QIQLVQSGPELKKPGETVKISCKASGYTFTNYGMN |
| | mAb | WVKQAPGKGLKWIGWINTYTGDPTYADDFKGRFA |
| | 27 | FSLETSASNGYLQIDNLKKEDTATYFCARRDIFAM |
| | | DYWGQGTSVTVSS |
| 61 | VL | DIQMTQTTSSLSASLGDRVTITCRASQDIMKYLHW |
| | mAb | YQQKPDGTIKLLFYYTSRLHSGVPSRFSGSGSGTDY |
| | 27 | SLTISNLEQEDIATYFCQQDVKYPWTFGGGTKLEIK |
| 62 | Pepsin- | MKWMVVVLVCLQLLEAAVVKVPLKKFKSIRETM |
| | ogen C | KEKGLLGEFLRTHKYDPAWKYRFGDLSVTYEPMA |
| | with | YMDAAYFGEISIGTPPQNFLVLFDTGSSNLWVPSVY |
| | tag | CQSQACTSHSRFNPSESSTYSTNGQTFSLQYGSGSL |
| | | TGFFGYDTLTVQSIQVPNQEFGLSENEPGTNFVYAQ |
| | | FDGIMGLAYPALSVDEATTAMQGMVQEGALTSPV |
| | | FSVYLSNQQGSSGGAVVFGGVDSSLYTGQIYWAPV |
| | | TQELYWQIGIEEFLIGGQASGWCSEGCQAIVDTGTS |
| | | LLTVPQQYMSALLQATGAQEDEYGQFLVNCNSIQ |
| | | NLPSLTFIINGVEFPLPPSSYILSNNGYCTVGVEPTYL |
| | | SSQNGQPLWILGDVFLRSYYSVYDLGNNRVGFATA |
| | | AAAGGGSHHHHHHHH |
| 63 | Pepsin- | MKWLLLLGLVALSECIMYKVPLIRKKSLRRTLSER |
| | ogen | GLLKDFLKKHNLNPARKYFPQWEAPTLVDEQPLE |
| | A5 | NYLDMEYFGTIGIGTPAQDFTVVFDTGSSNLWVPS |
| | with | VYCSSLACTNHNRFNPEDSSTYQSTSETVSITYGTG |
| | tag | SMTGILGYDTVQVGGISDTNQIFGLSETEPGSFLYY |
| | | APFDGILGLAYPSISSSGATPVFDNIWNQGLVSQDL |
| | | FSVYLSADDKSGSVVIFGGIDSSYYTGSLNWVPVT |
| | | VEGYWQITVDSITMNGETIACAEGCQAIVDTGTSLL |
| | | TGPTSPIANIQSDIGASENSDGDMVVSCSAISSLPDI |
| | | VFTINGVQYPVPPSAYILQSEGSCISGFQGMNVPTES |
| | | GELWILGDVFIRQYFTVFDRANNQVGLAPVAAAG |
| | | GGSHHHHHHHH |
| 64 | Pepsin- | MKWLLLLGLVALSECIMYKVPLIRKKSLRRTLSER |
| | ogen A | GLLKDFLKKHNLNPARKYFPQWEAPTLVDEQPLE |
| | | NYLDMEYFGTIGIGTPAQDFTVVFDTGSSNLWVPS |
| | | VYCSSLACTNHNRFNPEDSSTYQSTSETVSITYGTG |
| | | SMTGILGYDTVQVGGISDTNQIFGLSETEPGSFLYY |
| | | APFDGILGLAYPSISSSGATPVFDNIWNQGLVSQDL |
| | | FSVYLSADDKSGSVVIFGGIDSSYYTGSLNWVPVT |
| | | VEGYWQITVDSITMNGETIACAEGCQAIVDTGTSLL |
| | | TGPTSPIANIQSDIGASENSDGDMVVSCSAISSLPDI |
| | | VFTINGVQYPVPPSAYILQSEGSCISGFQGMNVPTES |
| | | GELWILGDVFIRQYFTVFDRANNQVGLAPVAAAG |
| | | GGS |

1. A kit comprising a first antibody or antigen binding fragment thereof and a second antibody or antigen binding fragment thereof,
a) wherein the first antibody or antigen binding fragment thereof is
an antibody or antigen binding fragment thereof that specifically binds to Pepsinogen C and competes for binding to Pepsinogen C with the antibody consisting of
(i) a heavy chain variable domain (VH) as shown in SEQ ID NO: 1; and
(ii) a light chain variable domain (VL) as shown in SEQ ID NO: 2; and
(b) wherein the second antibody or antigen binding fragment thereof is
an antibody or antigen binding fragment thereof that specifically binds to Pepsinogen C and competes for binding to Pepsinogen C, consisting of
(iii) a heavy chain variable domain (VH) as shown in SEQ ID NO: 9; and
(iv) a light chain variable domain (VL) as shown in SEQ ID NO: 10; and
wherein the second antibody or antigen binding fragments thereof does not compete for binding to Pepsinogen C with the first antibody or antigen binding fragment thereof.

2. The kit of claim 1, wherein the first antibody or antigen binding fragment comprises
(a) a VH comprising a CDR-H1 as shown in the amino acid sequence of SEQ ID NO: 3 or a variant thereof modified by one amino acid substitution; a CDR-H2 as shown in the amino acid sequence of SEQ ID NO: 4, or a variant thereof modified by one amino acid substitution; and a CDR-H3 as shown in the amino acid sequence of SEQ ID NO: 5, or a variant thereof modified by one amino acid substitution; and
a VL comprising a CDR-L1 as shown in the amino acid sequence of SEQ ID NO: 6 or a variant thereof modified by one amino acid substitution; a CDR-L2 as shown in the amino acid sequence of SEQ ID NO: 7, or a variant thereof modified by one amino acid substitution; and a CDR-L3 as shown in the amino acid sequence of SEQ ID NO: 52, or a variant thereof modified by one amino acid substitution; or
(b) a VH comprising a CDR-H1 as shown in the amino acid sequence of SEQ ID NO: 36 or a variant thereof modified by one amino acid substitution; a CDR-H2 as shown in the amino acid sequence of SEQ ID NO: 37, or a variant thereof modified by one amino acid substitution; and a CDR-H3 as shown in the amino acid sequence of SEQ ID NO: 38, or a variant thereof modified by one amino acid substitution; and
a VL comprising a CDR-L1 as shown in the amino acid sequence of SEQ ID NO: 39 or a variant thereof modified by one amino acid substitution; a CDR-L2 as shown in the amino acid sequence of SEQ ID NO: 40, or a variant thereof modified by one amino acid substitution; and a CDR-L3 as shown in the amino acid sequence of SEQ ID NO: 41, or a variant thereof modified by one amino acid substitution; and/or
wherein the second antibody, or antigen binding fragment thereof, comprises
(c) a VH comprising a CDR-H1 as shown in the amino acid sequence of SEQ ID NO: 11 or a variant thereof modified by one amino acid substitution; a CDR-H2 as shown in the amino acid sequence of SEQ ID NO: 12, or a variant thereof modified by one amino acid substitution; and a CDR-H3 as shown in the amino acid sequence of SEQ ID NO: 13, or a variant thereof modified by one amino acid substitution; and
VL comprising a CDR-L1 as shown in the amino acid sequence of SEQ ID NO: 14 or a variant thereof modified by one amino acid substitution; a CDR-L2 as shown in the amino acid sequence of SEQ ID NO: 15, or a variant thereof modified by one amino acid substitution; and a CDR-L3 as shown in the amino acid sequence of SEQ ID NO: 16, or a variant thereof modified by one amino acid substitution.

3. An antibody or antigen binding fragment thereof that specifically binds to Pepsinogen C and competes for binding to Pepsinogen C with the antibody consisting of
(i) a heavy chain variable domain (VH) as shown in SEQ ID NO: 1; and
(ii) a light chain variable domain (VL) as shown in SEQ ID NO: 2.

4. The antibody or antigen binding fragment thereof according to claim 3, comprising
(a) a VH comprising a CDR-H1 as shown in the amino acid sequence of SEQ ID NO: 3 or a variant thereof modified by one amino acid substitution; a CDR-H2 as shown in the amino acid sequence of SEQ ID NO: 4, or a variant thereof modified by one amino acid substitution; and a CDR-H3 as shown in the amino acid sequence of SEQ ID NO: 5, or a variant thereof modified by one amino acid substitution; and
a VL comprising a CDR-L1 as shown in the amino acid sequence of SEQ ID NO: 6 or a variant thereof modified by one amino acid substitution; a CDR-L2 as shown in the amino acid sequence of SEQ ID NO: 7, or a variant thereof modified by one amino acid substitution; and a CDR-L3 as shown in the amino acid sequence of SEQ ID NO: 52, or a variant thereof modified by one amino acid substitution; or
(b) a VH comprising a CDR-H1 as shown in the amino acid sequence of SEQ ID NO: 36 or a variant thereof modified by one amino acid substitution; a CDR-H2 as shown in the amino acid sequence of SEQ ID NO: 37, or a variant thereof modified by one amino acid substitution; and a CDR-H3 as shown in the amino acid sequence of SEQ ID NO: 38, or a variant thereof modified by one amino acid substitution; and
a VL comprising a CDR-L1 as shown in the amino acid sequence of SEQ ID NO: 39 or a variant thereof modified by one amino acid substitution; a CDR-L2 as shown in the amino acid sequence of SEQ ID NO: 40, or a variant thereof modified by one amino acid substitution; and a CDR-L3 as shown in the amino acid sequence of SEQ ID NO: 41, or a variant thereof modified by one amino acid substitution.

5. An antibody or antigen binding fragment thereof that specifically binds to Pepsinogen C and competes for binding to Pepsinogen C with the antibody consisting of
(i) a heavy chain variable domain (VH) as shown in SEQ ID NO: 9; and
(ii) a light chain variable domain (VL) as shown in SEQ ID NO: 10.

6. The antibody or antigen binding fragment thereof of claim 5, comprising
a VH comprising a CDR-H1 as shown in the amino acid sequence of SEQ ID NO: 11 or a variant thereof modified by one amino acid substitution; a CDR-H2 as shown in the amino acid sequence of SEQ ID NO: 12, or a variant thereof modified by one amino acid substitution; and a CDR-H3 as shown in the amino acid sequence of SEQ ID NO: 13, or a variant thereof modified by one amino acid substitution; and
a VL comprising a CDR-L1 as shown in the amino acid sequence of SEQ ID NO: 14 or a variant thereof modified by one amino acid substitution; a CDR-L2 as shown in the amino acid sequence of SEQ ID NO: 15, or a variant thereof modified by one amino acid substitution; and a CDR-L3 as shown in the amino acid sequence of SEQ ID NO: 16, or a variant thereof modified by one amino acid substitution.

7. The antibody or antigen binding fragment thereof according to claim 3, wherein the antibody or antigen binding fragment thereof binds to Pepsinogen C consisting of SEQ ID NO: 62 with a K_{D} of 2 nM or less, or 1.5 nM or less, or 1 nM or less.

8. A polynucleotide or a set of polynucleotides encoding
(i) the heavy chain variable domain of the antibody or antigen binding fragment thereof according to claim 3, and/or
(ii) the light chain variable domain of the antibody or antigen binding fragment according to claim 3.

9. A vector comprising the polynucleotide or the set of polynucleotides according to claim 8.

10. A host cell comprising the polynucleotide or the set of polynucleotides according to claim 8.

11. A method of producing the antibody or antigen binding fragment thereof according to claim 3 comprising culturing a host cell and isolating said antibody or antigen binding fragment thereof.

12. An antibody or an antigen binding fragment thereof obtainable by the method of claim 11.

13. A composition comprising
the antibody or antigen binding fragment thereof according to claim 3.

14. (canceled)

15. An in vitro method for detecting Pepsinogen C in a sample, comprising
(a) contacting a sample comprising Pepsinogen C with the antibody or antigen binding fragment thereof according to claim 3, thereby forming a complex comprising Pepsinogen C and said antibody or antigen binding fragment thereof, and
(b) detecting the complex formed in step (a), thereby detecting Pepsinogen C in said sample.

16. An in vitro method for detecting Pepsinogen C in a sample, comprising the following steps:
1) contacting a sample comprising Pepsinogen C with a first antibody or antigen binding fragment thereof and a second antibody or antigen binding fragment thereof, wherein
(a) the first antibody is
the antibody or antigen binding fragment thereof according to claim 3; and
(b) the second antibody specifically binds to Pepsinogen C and does not compete with the first antibody for specifically binding to Pepsinogen C;
2) incubating the resulting mixture of 1) to form a complex comprising Pepsinogen C and said first antibody or antigen binding fragment thereof and said second antibody or antigen binding fragment thereof and
3) detecting Pepsinogen C in the sample by detecting the formation of the complex as defined in 2).

17. The antibody or antigen binding fragment thereof according to claim 7, wherein the antibody or antigen binding fragment thereof binds to Pepsinogen C consisting of SEQ ID NO: 62 with a K_{D} of 0.6 nM or less.

18. The antibody or antigen binding fragment thereof according to claim 7, wherein the K_{D} is measured at 37 °C.
